SEQUENCE LISTING (12 sequences)

SEQ ID NO: 1 - RNA, 20 nt, synthetic construct
  misc_feature 1..20: note = Synthetic construct
  misc_feature 1..20: note = MISC_FEATURE - gRNA target for AAVS1
  source 1..20: mol_type = other RNA
ggggccactagggacaggat

SEQ ID NO: 2 - RNA, 20 nt, synthetic construct
  misc_feature 1..20: note = Synthetic construct
  misc_feature 1..20: note = MISC_FEATURE - gRNA target for ADORA2a-guide1
  source 1..20: mol_type = other RNA
gtctgtggccatgcccatca

SEQ ID NO: 3 - RNA, 20 nt, synthetic construct
  misc_feature 1..20: note = Synthetic construct
  misc_feature 1..20: note = MISC_FEATURE - gRNA target for ADORA2a-guide2
  source 1..20: mol_type = other RNA
tacaccgaggagcccatgat

SEQ ID NO: 4 - RNA, 20 nt, synthetic construct
  misc_feature 1..20: note = Synthetic construct
  misc_feature 1..20: note = MISC_FEATURE - gRNA target for CD73-guide1
  source 1..20: mol_type = other RNA
gcgggcgcccgcgcggctcg

SEQ ID NO: 5 - RNA, 20 nt, synthetic construct
  misc_feature 1..20: note = Synthetic construct
  misc_feature 1..20: note = MISC_FEATURE - gRNA target for CD73-guide2
  source 1..20: mol_type = other RNA
ctatgtgtccccgagccgcg

SEQ ID NO: 6 - RNA, 20 nt, synthetic construct
  misc_feature 1..20: note = Synthetic construct
  misc_feature 1..20: note = MISC_FEATURE - gRNA target for CD39
  source 1..20: mol_type = other RNA
tggcaccctggaagtcaaag

SEQ ID NO: 7 - protein, 363 aa, Homo sapiens
  REGION 1..363: note = MISC_FEATURE - ADA1 amino acid sequence
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 8 - protein, 511 aa, Homo sapiens
  REGION 1..511: note = MISC_FEATURE - ADA2 amino acid sequence
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 9 - protein, 70 aa, synthetic construct
  REGION 1..70: note = Synthetic construct
  REGION 1..70: note = MISC_FEATURE - CD8 transmembrane domain
TTTPAPRPPTPAPTIASQPLSLRPEACRPAAGGAVHTRGLDFACDIYIWAPLAGTCGVLLLSLVITLYCK

SEQ ID NO: 10 - DNA, 1176 bp, Homo sapiens
  misc_feature 1..1176: note = encodes the ADA1 sequence of SEQ ID NO: 7
  source 1..1176: mol_type = other DNA
atgttggtggatggcccatctgagcggccagccctgtgcttcttgctgttggctgtggcaatgtctttcttcggctcagctctatccatggcccaaaccccagcatttgataagcccaaagttgaacttcacgttcacctggatggttctataaagcctgaaaccattctgtactacgggagacggagagggattgcacttcctgccaatacagcagaaggccttctcaacgtaattggcatggacaagccacttactctccctgacttcttggcaaaatttgattactatatgccagcgatcgcgggttgcagggaggctatcaaacgcatcgcctatgagtttgtagagatgaaagcgaaggaaggtgtagtgtatgtggaggtccgctatagtcctcatcttctggcaaattctaaagtcgagcctataccctggaaccaggccgaaggggacctcactcctgatgaggttgtcgcactggtgggtcagggtctccaagagggggaaagagattttggcgttaaggctagaagtattttgtgttgcatgagacatcagccaaactggagcccgaaggtagttgagctgtgcaaaaaatatcaacaacagacggtggtcgctatcgacctcgcgggtgatgaaacaatccctggctccagtctgcttccagggcacgtccaagcataccaggaggctgtgaagtcaggcatacatagaaccgtgcatgccggagaggtagggagcgccgaggttgtaaaggaagctgtagacatactcaaaactgaacgccttggacacggttatcacacactcgaagaccaggcattgtataataggctccggcaagagaacatgcacttcgagatatgtccgtggtcctcctatttgacgggagcttggaagcccgatacggaacatgcggtgatccgactgaagaatgaccaggcgaactactccctgaacactgatgaccctttgatttttaaaagcacgttggatacagactaccaaatgactaaaagggatatgggttttactgaggaggagtttaagcggcttaacattaacgccgcaaagagctccttcctccctgaggacgaaaagcgcgaacttttggatttgctttacaaagcctatgggatgcctccaagcgcatccgcaggacaaaatctc

SEQ ID NO: 11 - DNA, 1533 bp, Homo sapiens
  misc_feature 1..1533: note = encodes the ADA2 sequence of SEQ ID NO: 8
  source 1..1533: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaggctgctgccggtgtatgagctcagtggagagcaccatgacgaagagtggtcagtgaagacttaccaggaagtagctcagaagtttgtggaaactcaccctgagtttattggaatcaaaatcatttattcggatcacagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaag

SEQ ID NO: 12 - DNA, 210 bp, synthetic construct
  misc_feature 1..210: note = Synthetic construct
  misc_feature 1..210: note = MISC_FEATURE - encodes the CD8 TMD sequence of SEQ ID NO: 7
  source 1..210: mol_type = other DNA
acaactacaccggctccacgacctcctaccccggcacccacaattgcatctcagccgctttccctgcggcctgaggcatgtagaccggccgcaggaggtgctgtacacacacgaggcttggacttcgcgtgtgatatatatatctgggctccactggcaggaacttgcggtgtgttgttgctgagtctcgtgattacactgtattgtaag

(19) United States
(12) Patent Application Publication
(10) Pub. No.: US 2025/0134998 A1
(43) Pub. Date: May 1, 2025

(54) T CELLS WITH CELL-SURFACE EXPRESSION OF ADENOSINE DEAMINASE AND USES THEREOF

(71) Applicant: The Board of Trustees of the Leland Stanford Junior University, Stanford, CA (US)

(72) Inventors: Dorota Danuta KLYSZ, Palo Alto, CA (US); Crystal L. MACKALL, Palo Alto, CA (US)

(21) Appl. No.: 18/687,634

(22) PCT Filed: Aug. 29, 2022
(86) PCT No.: PCT/US2022/075584

§ 371 Date: Feb. 28, 2024

Related U.S. Application Data
(60) Provisional application No. 63/238,756, filed on Aug. 30, 2021.

(51) Int. Cl. A61K 40/11; A61K 38/50; A61K 40/31; A61K 40/42; C07K 14/725; C07K 16/28; C12N 9/78; C12N 15/86

(52) U.S. Cl. CPC A61K 40/11; A61K 38/50; A61K 40/31; A61K 40/4211; A61K 40/4212; C07K 14/7051; C07K 16/2803; C12N 9/78; C12N 15/86; C12Y 305/04004; C07K 2317/31; C07K 2319/03; C12N 2740/10043

(57) ABSTRACT

The present disclosure relates generally to compositions and methods for robustly improving fitness and function of CAR T cells. More particularly, the present disclosure relates to novel chimeric polypeptides capable of anchoring adenosine deaminase activity to the surface of T cells, nucleic acids encoding the chimeric polypeptides, engineered T cells comprising the chimeric polypeptides or the nucleic acids encoding the chimeric polypeptides. Also provided herein are methods of generating the engineered T cells, methods of administering the engineered T cells, and methods of treating individuals of relevant health conditions.

### CROSS-REFERENCE TO RELATED APPLICATION

The present application claims priority to U.S. Provisional Patent Application Ser. No. 63/238,756, filed on Aug. 30, 2021, the disclosure of which is incorporated by reference herein in its entirety, including any drawings.

### INCORPORATION OF THE SEQUENCE LISTING

This application contains a Sequence Listing, which is hereby incorporated herein by reference in its entirety. The accompanying Sequence Listing text file, named “078430-535001WO_SequenceListing_ST26.xml,” was created on Aug. 25, 2022 and is 23 KB.

### BACKGROUND

Adoptive transfer of genetically modified immune cells, e.g., T cells, has emerged as a potent therapy for various malignancies. For example, current modalities of adoptive T cell therapy include cells modified to express receptors specific for cancer antigens, such as chimeric antigen receptors (CARs) and high-affinity T cell receptors (TCRs). Upon exposure to the cancer antigen, the modified T cells exhibit cytolytic activity and/or send signals to initiate an immune response against the cancer.

In adoptive T cell therapies, modified T cells are generally activated by exposure to the cognate antigen in vitro or ex vivo, expanded, and then administered to the subject, where they proliferate and have anticancer activity. Recent clinical trials using CAR-modified T cells (CAR− T cells) specific for the CD19 molecule on B-cell malignancies demonstrated marked disease regression in a subset of patients with advanced cancers. However, extending this therapy to other types of cancers, especially to solid tumors poses several challenges. For example, overstimulation due to prolonged antigen recognition and exposure to inflammatory signals can cause the T cells to lose effector function, a phenomenon called “T cell exhaustion.” Additionally, the tumor microenvironment elicits a number of tolerance and immunosuppression mechanisms that can reduce the effectiveness of adoptive cell therapies. For example, concentration of adenosine is one of the immunosuppressive mechanisms that CAR T cells need to face in the tumor microenvironment.

Thus, new compositions and strategies are needed for generating improved therapeutic cells for adoptive cell therapy. The presently disclosed aspects and embodiments address these needs and provide other related advantages.

### SUMMARY

Provided herein, inter alia, are novel methods and compositions for the prevention and/or treatment of various health conditions. In particular, described herein are immune cells, e.g., T cells, that have been engineered to express elevated levels of cell-surface expression of adenosine deaminase (ADA). In some embodiments, the engineered immune cells, e.g., engineered T cells, to immunosuppressive adenosine exhibit increased resistance and/or enhanced effector functions such as enhanced efficacy and persistence of T cells in patients. Also provided are methods for generating a population of engineered immune cells with enhanced effector function, and pharmaceutical compositions containing such a population of engineered immune cells with enhanced effector function, as well as methods and kits for the prevention and/or treatment of a health condition in subjects in need thereof.

In one aspect, provided herein are chimeric polypeptides including: (a) a first amino acid sequence comprising a first polypeptide module having adenosine deaminase activity, and (b) a second amino acid sequence comprising a second polypeptide module capable of anchoring the adenosine deaminase activity to a surface of a T cell.

Non-limiting exemplary embodiments of the disclosed chimeric polypeptides can include one or more of the following features. In some embodiments, the first polypeptide module is operably linked to the second polypeptide module. In some embodiments, the first polypeptide module having a human adenosine deaminase activity. In some embodiments, the human adenosine deaminase activity is of ADA1, ADA2, or a functional variant of any thereof. In some embodiments, the first polypeptide module comprises a amino acid sequence having at least 80% sequence identity to SEQ ID NO: 7 or SEQ ID NO: 8. In some embodiments, the second polypeptide module comprises a polypeptide transmembrane domain. In some embodiments, the polypeptide transmembrane domain is derived from a CD8, CD4, CD28, CD80, ICOS, CTLA4, PD1, PD-L1, BTLA, HVEM, CD27, 4-1BB, 4-1BBL, OX40, OX40L, DR3, GITR, CD30, SLAM, CD2, 2B4, TIM1, TIM2, TIM3, TIGIT, CD226, CD160, LAG3, LAIR1, B7-1, B7-H1, and B7-H transmembrane domain. In some embodiments, the polypeptide transmembrane domain is a CD8 transmembrane domain or a functional variant thereof. In some embodiments, the CD8 transmembrane domain comprises an amino acid sequence having at least 80% sequence identity to SEQ ID NO: 9.

In one aspect, provided herein are methods for generating an engineered T cell with enhanced effector function, the methods include introducing into a T cell a chimeric polypeptide as described herein, or nucleic acid encoding the chimeric polypeptide.

Non-limiting exemplary embodiments of the disclosed methods for generating an engineered T cell can include one or more of the following features. In some embodiments, the introduced chimeric polypeptide results in a reduced intracellular level of adenosine in the engineered T cell compared to reference T cell that does not comprise the chimeric polypeptide.

In some embodiments, the introduced chimeric polypeptide results in an enhanced effector function of the engineered T cell as compared to a control T cell, e.g., a T cell that has not been engineered to include such chimeric polypeptide. In some embodiments, the methods further include introducing into the T cell at least one recombinant antigen-specific receptor. In some embodiments, the at least one recombinant antigen-specific receptor includes an engineered T cell receptor (TCR) and/or an engineered chimeric antigen receptor (CAR).

In another aspect, some embodiments of the disclosure relate to engineered T cells that include a chimeric polypeptide including: (a) a first polypeptide module having adenosine deaminase activity; and (b) a second polypeptide module capable of anchoring the adenosine deaminase activity to a surface of a T cell. In a related aspect, some embodiments of the disclosure relate to engineered T cells that are produced by a method as described herein.

Non-limiting exemplary embodiments of the engineered T cells described herein can include one or more of the following features. In some embodiments, the T cell is a CD8+ T cytotoxic lymphocyte cell or a CD4+ T helper lymphocyte cell. In some embodiments, the CD8+ T cytotoxic lymphocyte cell is selected from the group consisting of naïve CD8+ T cells, central memory CD8+ T cells, effector memory CD8+ T cells, effector CD8+ T cells, CD8+ stem memory T cells, bulk CD8+ T cells. In some embodiments, the CD4+ T helper lymphocyte cell is selected from the group consisting of naïve CD4+ T cells, central memory CD4+ T cells, effector memory CD4+ T cells, effector CD4+ T cells, CD4+ stem memory T cells, and bulk CD4+ T cells. In some embodiments, the T cell is an exhausted T cell or a non-exhausted T cell.

In some embodiments, the T cell was obtained leukapheresis of a sample obtained from a subject.

In a related aspect, some embodiments of the disclosure relate to cell cultures including at least one engineered T cell of the disclosure and a culture medium.

In one aspect, provided herein are pharmaceutical compositions including an engineered T cell as disclosed herein and a pharmaceutically acceptable excipient.

In another aspect, provided herein are methods for preventing and/or treating a health condition in a subject in need thereof, the methods include administering to the subject a composition including: (a) at least one engineered T cells as described herein; and/or (b) a pharmaceutical composition as described herein.

Non-limiting exemplary embodiments of the methods of treatment described herein can include one or more of the following features. In some embodiments, the health condition is a proliferative disease (e.g., cancer), an autoimmune disease, or a chronic infection. In some embodiments, the subject is a mammalian subject. In some embodiments, the mammalian subject is a human subject. In some embodiments, the engineered T cells are autologous to the subject. In some embodiments, the engineered T cells are obtained from tumor infiltrating lymphocytes (TILs) or peripheral blood mononuclear cells (PBMCs). In some embodiments, the administered composition inhibits adenosine-mediated immunosuppression in the subject. In some embodiments, the administered composition confers an enhanced effector function of the engineered T cells as compared to control T cells under similar conditions, e.g., T cells that have not been administered with such composition. In some embodiments, the enhanced effector function of the engineered T cells is selected from the group consisting of growth rate (proliferation), death rate, death rate type, target cell inhibition (cytotoxicity), cluster of differentiation change, macrophage activation, B cell activation, cytokine production, in vivo persistence and increased spare respiratory capacity. In some embodiments, the enhanced effector function comprises increased production of one or more cytokines (e.g., interferon gamma (INFγ), tumor-necrosis factor α (TNFα), and interleukin-2 (IL-2)). In some embodiments, the composition is administered to the subject individually (monotherapy) or in combination with a second therapy, wherein the second therapy is selected from the group consisting of chemotherapy, radiotherapy, immunotherapy, hormonal therapy, toxin therapy, or surgery.

In another aspect, some embodiments of the disclosure relate to kits for the prevention and/or treatment of a condition in a subject in need thereof, the kits include one or more of the following: (a) a chimeric polypeptide as described herein; (b) nucleic acid encoding the chimeric polypeptide as described herein; (c) at least one engineered T cells as described herein; and (d) a pharmaceutical composition as described herein.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1E summarize the results of experiments performed to illustrate that CD39 expression marks highly dysfunctional population of exhausted CART T cells that exhibits suppressive capacity.

FIG. 1A illustrates dynamic of surface CD39 expression on CD19.28z and exhausted HA CAR T cells. Representative donor of n=3

FIG. 1B illustrates IL-2 and IFNγ secretion by CD19.28z and HA CAR T cell stimulated with Nalm6-GD2 tumor cell line at Day 6 and 14 post T-cell activation. Data are mean±s.e.m. from triplicate wells. Representative donor of n=3

FIG. 1C illustrates cytokines secreted by CD39+ vs CD39-CD8 HA CAR T cells 24 hrs post-stimulation with Nalm6-GD2+ cells detected by Luminex (pg/ml). Data are mean of n=3 donors.

FIG. 1D illustrates CyTOF analysis of HA CAR T cells at Day 10 post-activation. Heat map represents median expression of indicated markers in CD4 or CD8 CD39− and CD39+. Heat map was generated using median Arcsinh ratio of given marker to the total CD8 value in each column. Representative donor shown (n=3 donors).

FIG. 1E is illustrates GSEA of indicated signatures from the ranked list of differentially expressed genes in CD8+CD39+ versus CD8+CD39−.

FIG. 1F is illustrates CD19BB CAR T cells were activated with Nalm6 tumor line alone or in the presence of total HA or CD8 purified HA CAR T cells. IL-2 secretion by CD19BB CAR T cell was assessed 24 hrs post-stimulation. Data are mean±s.e.m. from triplicate wells. Representative experiment of n=2 shown.

FIGS. 2A-2C summarize the results of experiments performed to illustrate that HA.28z (HA) CAR T cells exhibit higher expression of exhaustion markers and decreased cytokine secretion as compared to CD19.28z CAR T cells.

FIG. 2A illustrates expression of exhaustion markers at Day 14 post T-cell activation in non-transduced Mock (grey), CD19.28z (blue) or HA (red) CAR T cells and (B) IL-2 (left) and IFNγ (right) release after 24 h co-culture with CD19+GD2+ Nalm6-GD2 leukemia cells. Data are mean±s.e.m. from triplicate wells from 3-4 donors

FIG. 2B illustrates contour plots showing kinetics of expression of exhaustion markers upon activation in CD19.28z and HA CAR T cells.

FIG. 2C illustrates MFI and frequency of exhaustion markers expressed by CD19.28z and HA CAR T cells at indicated time points post-activation. C. MFI and frequency of exhaustion markers expressed by CD19.28z and HA CAR T cells at indicated time points post-activation.

FIGS. 3A-3C summarize the results of experiments performed to characterize CD39+CD4 HA CAR T cells.

FIG. 3A illustrates percentage of CD39+ cells in CD4+ vs CD8+ compartment of HA CAR T cells. n=11 donors from independent experiments. Contour plots of one representative donor shown.

FIG. 3B illustrates cytokines secreted by CD39+ vs CD39-CD4 HA CAR T cells 24 hrs post-stimulation with Nalm6-GD2+ cells detected by Luminex. Data are mean of 3 donors.

FIG. 3C illustrates CyTOF analysis of CD4 HA CART cells at Day 10 post-Activation. Heat map represents median expression of indicated markers in CD4 or CD8 CD39− and CD39+. Heat map was generated using median Arcsinh ratio of given marker to the total CD4 or CD8 value in each column. Representative donor shown (n=3).

FIGS. 4A-4B summarize the results of experiments performed to illustrate transcriptional differences between CD39+ versus CD39− CD8 and CD4 HA CAR T cells.

FIG. 4A illustrates volcano plot depicting RNA expression level in CD39+ T cells relative to CD39− T cells. Significantly different genes were identified by DESeq2 (Wald test) and are shown in red (adjusted P value <0.1).

FIG. 4B illustrates Venn diagram depicting the overlap between 95 genes that were significantly upregulated in CD4+CD39+ T cells relative to CD4+CD39− T cells and 57 genes that were significantly upregulated in CD8+CD39+ T cells relative to CD8+CD39− T cells.

FIGS. 5A-5C summarize the results of experiments performed to illustrate that conversion of CD39− into CD39+ CAR T cells depends on tonic signaling of CAR and does not require TGFβ.

FIG. 5A illustrates percentage of CD39+ in CAR+ vs CAR− HAT cells at Day 10 post-activation. N=12 donors from independent experiments.

FIG. 5B illustrates that at Day 16 post-activation bulk HA or purified CD39− HA CAR T cell population was cultured in the presence of 1 μM dasatinib or 0.001 mg/ml neutralizing anti-TGFβ. 7 days post-treatment CD39 expression was evaluated. Data are mean±s.e.m. of 3-4 donors from independent experiments. P values determined by unpaired two-tailed t-tests.

FIG. 5C illustrates that at Day 16 post-activation bulk HA or purified CD39− HA CAR T cell population was cultured in the presence of 1 μM dasatinib or 0.001 mg/ml neutralizing anti-TGFβ. 7 days post-treatment CD39 expression was evaluated. Dot plots of representative donor shown. Data are mean±s.e.m. of 3-4 donors from independent experiments. P values determined by unpaired two-tailed t-tests.

FIGS. 6A-6E summarize the results of experiments performed to illustrate that exhausted CAR T cells can convert ATP into immunosuppressive adenosine through CD39 and CD73 expression, or exhausted HA CAR T cells exhibit high expression of enzymatically active CD39 and CD73 which results in adenosine production.

FIG. 6A is a schematic of purinergic pathway.

FIG. 6B illustrates representative counter plots showing expression of CD39 and CD73 by HA CAR T cells at Day 14 post-activation (left). Percentage of double positive CD39+CD73+ T cells in CD4+ vs CD8+HA CAR T cells (right) in 4 donors from independent experiments.

FIG. 6C illustrates percentage of eATP hydrolysis by CAR T cells pre-incubated or not with and anti-CD73 inhibiting antibody (left: representative data from n=4 donors) and percentage of eADO produced by ecto-nucleotidases expressed on the surface of CAR T cells (right: representative data from n=3 donors).

FIG. 6D illustrates IL-2 secretion by HA and CD19 CAR T cells stimulated with Nalm6GD2 cell line in the presence or absence of NECA or 40 μM of a2aR inhibitor at Day 16 post-activation. Data are mean±s.e.m. from triplicate wells. Representative data of n=2 donors.

FIG. 6E illustrates CD19bb or CD19bb A2aR knock out CAR T cells were activated with Nalm6 tumor line alone or in the presence of total HA or HA with CD39 knock out. IL-2 secretion by CD19bb CAR T cell was assessed 24 hrs post-stimulation.

FIGS. 7A-7D summarize the results of experiments performed to illustrate that both exhausted and non-exhausted CAR T cells cytokine suppression can be mediated by NECA in dose-responsive manner.

FIG. 7A illustrates IL-2 and IFNγ secretion by HA CAR T cells stimulated with Nalm6-GD2 cell line at Day 10 or 16 in the presence of different concentration of NECA and with or without 40 μM of A2aR inhibitor CPI444. Data are mean±s.e.m. from triplicate wells. Representative donor of 3 independent experiments.

FIG. 7B illustrates IL-2 and IFNγ secretion by CD19 CAR T cells stimulated with Nalm6-GD2 cell line at Day 10 or 16 in the presence of different concentration of NECA and with or without 40 μM of A2aR inhibitor CPI444. Data are mean±s.e.m. from triplicate wells. Representative donor of 3 independent experiments.

FIG. 7C illustrates NFkB-GFP activation level 4 hrs after stimulation of HA CAR T cells in the presence or absence of NECA and A2aR competitive inhibitor. Data are mean s.e.m. from triplicate wells.

FIG. 7D illustrates adenosine secretion by the cells after mock or CAR T cells were incubated for 2 hrs. Representative of 2 experiments.

FIGS. 8A-8D summarize the results of experiments performed to illustrate that ATP metabolism affects exhausted CAR T cell phenotype and function.

FIG. 8A illustrates expanded phenotyping of genetically modified HA CAR T cells. Approximately 5,000 or maximum of CD8+HA CAR T cells from each donor (n=4) were organized by their combined expression of 26 markers using UMPAS. Donors matched by the deleted gene and concatenated into 93,280 randomly-sampled total events.

FIG. 8B illustrates phenotypical clusters defined using FlowSOM algorithm.

FIG. 8C illustrates IL-2 and IFNγ secretion by genetically modified and idiotype-stimulated HA CAR T cells in the presence of 100 μM of ATP 24 hrs post-stimulation

FIG. 8D illustrates co-cultures with Nalm6-GD2 (1:8 E:T), mg63.3 (1:5 E:T) or 143b (1:1 E:T) tumor lines respectively in an IncuCyte to assess CAR-T cell cytotoxicity. Tumor GFP fluorescence intensity was normalized to the first time point (duplicate or triplicate wells). Representative donor of n=2-3 shown.

FIGS. 9A-9D summarize the results of experiments performed to illustrate that manipulating purinergic pathway affects exhausted CAR T cell phenotype and function.

FIG. 9A illustrates heat map representing Arhin median expression of 26 markers used for FlowSOM analysis manually gated on CD8 CAR T cells. Samples from four donor were concatenated using OMIQ platform.

FIG. 9B illustrates CD4 HA CAR T cells from four donors (5,000 events per sample or maximum) organized by their combined expression of 26 markers using UMPAS and colored by cluster ID defined by FlowSOM. Donors matched by the deleted or overexpressed gene and concatenated into 93,280 randomly-sampled total events

FIG. 9C illustrates IL-2 and IFNγ secretion by HA CAR T cells stimulated with Nalm6-GD2 in the presence of 200 μM ATP in order to mimic solid tumor microenvironment

FIG. 9D illustrates histograms representing levels of expression of GD2 antigen on various tumor lines.

FIGS. 10A-10H summarize the results of experiments performed to illustrate that overexpression of adenosine deaminase ADA1 improves exhausted and non-exhausted CAR T cell function.

FIG. 10A is a schematic description of ADA1 role in purinergic pathway (top) and ADA1 construct structure (bottom).

FIG. 10B illustrates expanded phenotyping of genetically modified HA CAR T cells at Day 14 post-activation. Five thousands or maximum of CD8+HA CAR T cells from each donor (n=4) were organized by their combined expression of 26 markers using UMPAS. Donors matched by the deleted or overexpressed gene and concatenated into 93,280 randomly-sampled total events. Violin plots showing frequency of population clusters defined using FlowSOM per condition.

FIG. 10C illustrates a plot of principal components 1 vs 2 for each of the expression profile assessed at Day 14 post-activation in cells from CRISPR-knockout experiment (KO) or ADA-overexpressing (O/E) HA CAR T cells.

FIG. 10D illustrates a volcano plot depicting RNA expression level in control HA T cells relative to ADA O/E HA CAR T cells. Significantly different genes were identified by DESeq2 (Wald test) (adjusted P value <0.01).

FIG. 10E is a heatmap showing DEGs identified from AAVS1 vs ADA overexpressing HA CAR T cells.

FIG. 10F illustrates 02 consumption rates (OCR) and extracellular acidification rate (ECAR) for HA and ADA O/E CAR T cells measured by Seahorse at Day 11 post-activation. Bar graphs show quantitative data of spare respiratory capacity (SRC) and basal OCR/ECAR ratio. Representative plots shown (n=2).

FIG. 10G illustrates frequency of Foxp3+CD25+CD127− CD8 HA and HA ADA O/E CAR T cells at Day 10 post-activation.

FIG. 10H illustrates cytotoxic function at 1:8 E:T ratio, IL-2 secretion after 24 hrs stimulation and proliferation index 72 hrs after stimulation with Nalm6-GD2 or CHLA25.5 tumor lines. Data are mean±s.e.m. from duplicate or triplicate wells. Representative graph shown (n=2-3 donors).

FIGS. 11A-11G summarize the results of experiments performed to illustrate expressions of CD39 and CD73 on different tumor lines and effect of overexpression of ADA on CAR T cell phenotype, transcriptome, and function.

FIG. 11A illustrates CD39 and CD73 surface expression by various tumor cell lines.

FIG. 11B illustrates ADA-Tag surface expression and CAR expression.

FIG. 11C illustrates expression of CD69 after 24 hrs of HA CART cell co-cultured with Nalm6-GD2+ leukemia cells in the presence or absence of 200 μM eATP. Data are mean s.e.m. from triplicate wells.

FIG. 11D illustrates phenotype and frequency of indicated population in CD4+HA and HA ADA O/E CAR T cells analyzed using FlowSOM at Day 14 post-activation.

FIG. 11E illustrates GSEA analysis of HA ADA O/E vs HA CAR T cells at Day 14 post-activation.

FIG. 11F illustrates killing Incucyte assay at 1:8 E:T ratio, IL-2 secretion 24 hrs post-stimulation and proliferation index 72 hrs after stimulation with Nalm6 tumor line at Day 14 post-activation.

FIG. 11G illustrates killing Incucyte assay at 1:8 E:T ratio, IL-2 secretion 24 hrs post-stimulation and proliferation index 72 hrs after stimulation with Nalm6 tumor line at day 14 post-activation.

FIG. 12A illustrates two exemplary plasmid designs for expression of ADA1 and ADA2. SS: signaling domain (leader sequence); CD8-TM: CD8 transmembrane sequence; tEGFR: surface selection marker (truncated EGFR-like protein).

FIG. 12B schematically summarizes the results of experiments performed to demonstrate that overexpression of each of the two human ADA isoenzymes resulted in an increased tumor killing by the HA CAR T cells.

FIG. 12C schematically summarizes the results of experiments performed to demonstrate that non-exhausted, CAR T cells expressing membrane bound ADA1 or ADA2 secreted higher levels of IL-2 and IFNγ.

FIG. 12D schematically summarizes the results of experiments performed to demonstrate that CAR T cells expressing ADA1-TM or ADA2-TM produced more IL-2 and IFNγ as compared to control group and this increase was abrogated in the presence of adenosine deaminase inhibitor—EHNA. HA CAR T cells were stimulated with Nalm6-GD2 tumor line in the presence or absence of 10 uM of EHNA inhibitor at day 15 post-activation. IL-2 and IFNγ secretion was assessed using ELISA. One donor shown.

FIG. 12E schematically summarizes the results of experiments performed to test anti-tumor potency of transmembrane-bound adenosine deaminase by using 143b solid tumor model.

### DETAILED DESCRIPTION OF THE DISCLOSURE

The present disclosure generally relates to, inter alia, methods and compositions for the prevention and/or treatment of various health conditions. In particular, described herein are chimeric polypeptides having adenosine deaminase activity for increasing effector function of CAR T cells. Also provided are methods for generating engineered immune cells with enhanced effector function, the engineered immune cells according to the presently described methods, pharmaceutical compositions comprising the same, as well as methods and kits for the prevention and/or treatment of a health condition in subjects in need thereof.

The following descriptions and examples illustrate embodiments of the present disclosure in detail. Although the present disclosure has been described in some details by way of illustration and example for purposes of clarity and understanding, it will be apparent that certain changes and modifications can be practiced within the scope of the appended claims.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment. It is to be understood that the present disclosure is not limited to the particular embodiments described herein and as such can vary. Those of skill in the art will recognize that there are variations and modifications of the present disclosure, which are encompassed within its scope.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the disclosure can be used in combination with any other unless specifically indicated otherwise.

### Definition

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated cases, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a” “an”, and “the” include plural referents unless the context clearly dictates otherwise. For example, the term “a cell” includes one or more cells, including mixtures thereof “A and/or B” is used herein to include all of the following alternatives: “A”, “B”, “A or B”, and “A and B”.

Furthermore, the use of the term “including” as well as other forms, such as “include”, “includes” and “included”, is not limiting.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the present disclosure can be used to achieve methods of the present disclosure.

The term “administration” and its grammatical variations, as used herein, refer to the delivery of a bioactive composition or formulation by an administration route comprising, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

The term “cancer” refers to the presence of cells possessing several characteristics of cancer-causing cells, such as uncontrolled proliferation, immortality, metastatic potential, rapid growth and proliferation rate, and certain characteristic morphological features. Cancer cells can aggregate into a mass, such as a tumor, or can exist alone within a subject. A tumor can be a solid tumor, a soft tissue tumor, or a metastatic lesion. As used herein, the term “cancer” also encompasses other types of non-tumor cancers. Non-limiting examples include blood cancers or hematological cancers, such as leukemia. Cancer can include premalignant, as well as malignant cancers.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications can occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “operably linked”, as used herein, denotes a physical or functional linkage between two or more elements, e.g., polypeptide sequences or polynucleotide sequences, which permits them to operate in their intended fashion. For example, an operably linkage between a polynucleotide of interest and a regulatory sequence (for example, a promoter) is functional link that allows for expression of the polynucleotide of interest. It should be understood that, operably linked elements may be contiguous or non-contiguous. In the context of a polypeptide, “operably linked” refers to a physical linkage (e.g., directly or indirectly linked) between amino acid sequences (e.g., different domains) to provide for a described activity of the polypeptide. In the present disclosure, various domains of the recombinant polypeptides of the disclosure may be operably linked to retain proper folding, processing, targeting, expression, binding, and other functional properties of the recombinant polypeptides in the cell. Operably linked domains of the recombinant polypeptides of the disclosure may be contiguous or non-contiguous (e.g., linked to one another through a linker).

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a sequence. This definition also includes sequences that have deletions and/or additions, as well as those that have substitutions. Sequence identity typically is calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “recombinant” or “engineered” nucleic acid molecule, polypeptide, or cell as used herein, refers to a nucleic acid molecule, polypeptide, or cell that has been altered through human intervention.

As used herein, and unless otherwise specified, a “therapeutically effective amount” or a “therapeutically effective number” of an agent is an amount or number sufficient to provide a therapeutic benefit in the treatment or management of a disease, e.g., cancer, or to delay or minimize one or more symptoms associated with the disease. A therapeutically effective amount or number of a compound means an amount or number of therapeutic agent, alone or in combination with other therapeutic agents, which provides a therapeutic benefit in the treatment or management of the disease. The term “therapeutically effective amount” can encompass an amount or number that improves overall therapy of the disease, reduces or avoids symptoms or causes of the disease, or enhances therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human subject) and non-human animals. In some embodiments, a “subject” or “individual” is a patient under the care of a physician. Thus, the subject can be a human patient or a subject who has, is at risk of having, or is suspected of having a disease of interest (e.g., cancer) and/or one or more symptoms of the disease. The subject can also be a subject who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

As used herein, the term “functional variant thereof” relates to a molecule having qualitative biological activity in common with the wild-type molecule from which the variant was derived. For example, when referencing a polypeptide having an enzymatic activity (e.g., an enzyme such as an adenosine deaminase; ADA), the term “functional variant” refers to an enzyme that has a polypeptide sequence that is at least about 70%, at least about 75%, at least about 80%, at least about 85%, at least about 90%, at least about 95% or at least about 99% identical to a polypeptide sequence encoding the enzyme. The “functional variant” enzyme may retain amino acids residues that are recognized as conserved for the enzyme, and may have non-conserved amino acid residues substituted or found to be of a different amino acid, or amino acid(s) inserted or deleted, but which does not affect or has insignificant effect its enzymatic activity, as compared to the enzyme described herein. The “functional variant” enzyme has an enzymatic activity that is identical or essentially identical to the biological activity of the enzyme (e.g., ADA) described herein. One skilled in the art will appreciate that the “functional variant” enzyme may be found in nature, i.e., naturally occurring, or be an engineered mutant thereof. As such, the term “ADA polypeptide variant” includes naturally occurring allelic variants or alternative splice variants of an ADA polypeptide. For example, an ADA polypeptide variant includes the substitution of one or more amino acids in the amino acid sequence of a parent ADA polypeptide with a similar or homologous amino acid(s) or a dissimilar amino acid(s). There are many scales on which amino acids can be ranked as similar or homologous. (Gunnar von Heijne, Sequence Analysis in Molecular Biology, p. 123-39 (Academic Press, New York, NY 1987).

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Headings, e.g., (a), (b), (i) etc., are presented merely for ease of reading the specification and claims. The use of headings in the specification or claims does not require the steps or elements be performed in alphabetical or numerical order or the order in which they are presented.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to”, “at least”, “greater than”, “less than”, and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to +5%, or up to +1%.

It is understood that aspects and embodiments of the disclosure described herein include “comprising,” “consisting,” and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub combination was individually and explicitly disclosed herein.

### Adenosine Deaminase and Car T Cell Therapy

### Adenosine Deaminase

Adenosine deaminase (also known as adenosine aminohydrolase, or ADA) is an enzyme (EC 3.5.4.4) considered one of the key enzymes of purine metabolism. The enzyme has been found in bacteria, plants, invertebrates, vertebrates, and mammals, with high conservation of amino acid sequence. The high degree of amino acid sequence conservation suggests the crucial nature of ADA in the purine salvage pathway.

Primarily, ADA in humans is involved in the development and maintenance of the immune system. However, ADA association has also been observed with epithelial cell differentiation, neurotransmission, and gestation maintenance. It has also been proposed that ADA, in addition to adenosine breakdown, stimulates release of excitatory amino acids and is necessary to the coupling of A1 adenosine receptors and heterotrimeric G proteins. Adenosine deaminase deficiency leads to pulmonary fibrosis, suggesting that chronic exposure to high levels of adenosine can exacerbate inflammation responses rather than suppressing them. It has also been recognized that adenosine deaminase protein and activity is upregulated in mouse hearts that overexpress HIF1α, which in part explains the attenuated levels of adenosine in HIF-1α expressing hearts during ischemic stress.

Two types of adenosine deaminases exist in humans: ADA1 and ADA2. ADA1 (41 kDa) is encoded by the ADA gene on chromosome 20q13.12 (OMIM 608958 or Entrez Gene ID 100) and is produced by all cells. The primary role of ADA1, which acts as a monomer, is to eliminate intracellular toxic derivatives of adenosine and deoxyadenosine and to protect the cells from apoptosis. The absence of ADA1 due to genetic mutations results in severe combined immunodeficiency (SCID). While the intracellular role of ADA1 has been established, this enzyme also has extracellular roles, including formation by ADA1 (or ecto-ADA) of a ternary complex with CD26 and A2a receptors bridging two different cells as a co-stimulatory molecule that impacts T-cell proliferation. ADA1 converts adenosine, an endogenous purine metabolite that acts via leukocyte purine receptors to suppress pro-inflammatory and Th1-polarizing responses, to inosine, which is immunologically inert. ADA1 also has roles in enhancing T-helper 2 (Th2) immunity via adenosine receptors. ADA1 deficiency impairs thymocyte development and B-lymphocyte immunoglobulin production resulting in severe combined immunodeficiency.

ADA2 (57 kDa) is encoded by the CECR1 (ADA2) gene on chromosome 2211.1 (OMIM 607575 or Entrez Gene ID 51816) and is produced by activated monocytes, macrophages, and dendritic cells (DCs). Independent of its enzymatic activity, ADA2 modulates immunity via binding cognate receptors on immune cells. ADA2 also induces monocyte differentiation to macrophages in T-cell co-cultures. ADA2, also known as Cat Eye Syndrome Chromosome Region, Candidate 1, or CECR1, is an adenosine deaminase that catalyzes the deamination of adenosine and 2-prime-deoxyadenosine to inosine and deoxyinosine, respectively. In contrast to ADA1, ADA2 is a secreted homodimer and is highly expressed in plasma. ADA2 is highly expressed in dendritic cells, CD14+ monocytes, and lymphoid tissues, particularly in the thymus. ADA2 has a higher Km for adenosine (23, 24) and is thereby less enzymatically active than ADA1. While residual ADA2 activity ADA2 can be measured in patients with ADA1 deficiency (23, 25), its important roles in immunity has previously been under-appreciated.

### CAR T Cell Therapy

CAR T cell therapy has been shown to be highly effective in many types of blood cancers. However, in the context of solid tumors, there are still many challenges to address before CAR T can become a standard treatment, including overcoming the hostile tumor microenvironment has been proven to be one of the most challenging. In this context, several reports have highlighted the role of adenosine as a crucial immunosuppressive factor that accumulates in the tumor microenvironment. In a pathological condition, extracellular adenosine (eADO) concentration can be up to 100 times higher than in a physiological state. This increase can be the result of passive release of adenosine from dying cells, active export through equilibrative nucleoside transporters (ENTs), or production of adenosine through ATP/ADP catabolism mediated by CD39 and CD73. Extracellular adenosine then can subsequently suppress T cells through interaction with different G protein-coupled receptors: ADORA1, ADORA2A, ADORA2B, ADORA3, where ADORA2a (A2aR) has the highest affinity. Therefore, targeting adenosinergic pathway represents an attractive new therapeutic strategy in the context of CAR T cell immunotherapy.

As described in greater details below, experimental data presented herein demonstrate that, inter alia, a population of exhausted CAR T cells exhibiting antigen-independent clustering has high surface expression of ectoenzymes CD39 and CD73, both of which are involved in adenosine production, and that exhausted CD39+ CAR T cells upregulate markers associated with Treg phenotype at the gene and protein levels and exhibit adenosine-mediated suppressive functions. These data further support the different expression kinetics of CD39 compared to canonical exhaustion/activation markers, such as TIM3, LAG3 or PD1, which are upregulated early upon activation. Unlike these canonical exhaustion/activation markers, CD39 appear to correlate specifically with progressive loss of function in exhausted CAR T cells.

CD39+CD8 exhausted CAR T cells exhibit a distinctive transcriptional, phenotypic, and functional profile as compared to their CD39− counterparts. CD39+CD8 CAR T cells exhibit high proliferative potential as defined as Ki67+ and secrete elevated levels of cytokines, such as IFNγ, granzyme B, IL-27, and TGFβ, which are involved in Treg differentiation and function. They also secrete low level of IL-2, MPC-1, TNFα, and TNFβ cytokines. These data suggest a high level of similarity between CD39+ exhausted and regulatory T cells. The gene enrichment analysis confirmed upregulation of genes associated with the Treg phenotype. Additionally, CD39+CD8 CAR T cells show a consistent suppressive capacity via adenosine production which aligns with reports characterizing CD39 as a Treg marker and in vitro suppressive function of CD39+CD8 T cells obtained from human tumors. However, in contrast to studies showing that CD73 expression is reduced upon activation and differentiation, demonstrated herein is that exhausted CD8 CAR T cells exhibit increased expression of both CD39 and CD73, which leads to active hydrolysis of eATP and generation of adenosine.

It has been shown that TGFβ is linked to CD39 expression. However, as demonstrated herein, neutralizing TGFβ antibodies does not cause any changes in CD39 expression in HA CAR T cells. Instead, the experimental data presented herein demonstrate a strong correlation between CAR expression and CD39 upregulation, suggesting the role of intrinsic factors. Indeed, blocking tonic signaling with dasatinib (tyrosine kinase inhibitor) in CD39− sorted HA CAR T cells inhibits CD39 expression. This supports that when cells are in a state of chronic antigen stimulation, increased suppressive capacity of the cells is necessary to combat overstimulation and self-damage.

As demonstrated herein, deletion of adenosine receptor A2aR in exhausted CAR T cells, in contrast to deletion of CD39 or CD73, does not result in significant phenotypic changes, but improves tumor-specific killing. Of CAR T cells with knockouts of CD39, CD73, and high affinity adenosine A2a receptor, only CD39 or CD73 knockouts affect exhaustion phenotype, leading to significantly increased frequency of Tscm and effector-like population of CAR T cells. Despite these changes in the phenotype, deletion of all three genes leads to increased IL-2 secretion in short time course assays. Only A2aR KO exhibits significantly higher tumor growth control in the killing test.

A2a receptor plays a crucial role in inosine-induced anti-tumor T cell response. Moreover, A2b receptor can be expressed by T cells and mediate T cell suppression. Therefore, A2aR knock out may not be the most efficient way to improve CAR T cell therapy. Instead, overexpression of adenosine deaminase (ADA) enzyme on the surface of CAR T cells can decrease adenosine accumulation in the tumor microenvironment. Thus, in an aspect, provided herein are compositions and methods for increasing effector function of CAR T cells by overexpressing ADA, which can modulate balance between adenosine and non-suppressive inosine, less potent agonist of A2aR than adenosine. Adenosine can increase a cAMP-biased signaling while inosine activates ERK1/2-biased signaling. Inosine signaling through A2aR has been shown to induce Th1-type response that is beneficial in the context of T cell immunotherapy. Inosine can be utilized by T cells as an alternative to glucose as a source of energy and support CAR T cell effector function. As demonstrated herein, ADA overexpression in CAR T cells induces changes at the transcriptional and protein level, exhibiting more T_{SCM} (stem cell memory T cell)-like and less exhausted phenotype. ADA overexpression in both exhausted and non-exhausted CAR T cells lead to changes in phenotype, with a higher frequency of stem cell-like memory T cell effectors and a simultaneous decrease of exhausted sub-populations. Both antigen-driven proliferation and effector function of CAR T cells significantly improve after ADA overexpression. Memory cells have been reported to exhibit increased β-oxidation and mitochondrial spare respiratory capacity (SRC). As demonstrated herein, ADA-overexpressing CAR T cells are enriched in expression of genes involved in fatty acid metabolism and show a significant shift towards oxidative phosphorylation and enhanced SRC, as compared to control CAR T cells under similar conditions, e.g., CAR T cells that have not been engineered to overexpress such ADA. This translates into improved antigen-specific proliferation and cytotoxic function. This phenomenon is observed in both exhausted and non-exhausted CAR T cells.

Experimental data presented herein demonstrate that overexpression of adenosine deaminase (ADA) can be an innovative approach to increase effector function of CAR T cells by modulating balance between adenosine and non-suppressive inosine. In some embodiments, the ADA activity is of ADA1, ADA2, or a functional variant of any thereof.

### Compositions of the Disclosure

As described in greater details below, one aspect of the present disclosure relates to a chimeric polypeptide comprising one or more polypeptide modules, for example, having a first polypeptide module with adenosine deaminase activity and a second polypeptide module capable of anchoring the adenosine deaminase activity to a surface of a T cell. Some embodiments of the present disclosure provide an engineered T cell comprising the chimeric polypeptide or a nucleic acid encoding the chimeric polypeptide.

### Chimeric Polypeptides

As outlined above, some embodiments of the present disclosure relate to a chimeric polypeptide engineered to improve CAR T cells phenotype and effector function. As described in greater details in Examples section below, CD39 expression correlate with progressive loss of function during CAR T cell exhaustion. Briefly, CD39+CD8+ exhausted CAR T cells exhibit Treg-associated phenotype and suppression function. CD39+CD8 CAR T cells are not only exhausted, but they can represent a novel cell subpopulation with enriched suppressive molecular signature, phenotype and function. As explained in greater details below, conversion of CD39-CAR T cell population into CD39+ depends on tonic signaling. Chronic T cell stimulation is sufficient to convert CD39− cells into CD39+ cells. Exhausted CAR T cells exhibit high expression of enzymatically active CD39 and CD73 which results in production of suppressive adenosine. Higher levels of expression of CD39 and CD73 on the surface of exhausted CAR T cells correlate with increased capacity to degrade ATP and transform ADP/AMP into adenosine.

As demonstrated herein, adenosine can suppress cytokine production by CART T cells after antigen stimulation. Exhausted CAR T cells express active CD39 and CD73 on their surface, which results in increased capability of generating adenosine. Adenosine in turn exerts suppressive effects on CAR T cell function and proliferation in an A2a receptor-mediated manner. Blocking A2a receptor on CAR T cells or knocking-out CD39 on CAR T cells can restore cytokine (e.g., IL-2) production by CAR T cells in the presence of adenosine-producing cells. Therefore, purinergic pathway can modulate exhausted CAR T cells phenotype and function. Autocrine adenosine production, as a result of co-expression of CD39 and CD73 by CAR T cells, can lead not only to suppressive effects on neighboring cells, but to intrinsic suppression of CAR T cell activity. Adenosine and the pathway that regulates its production play a crucial role in modulating CAR T cell response and phenotype.

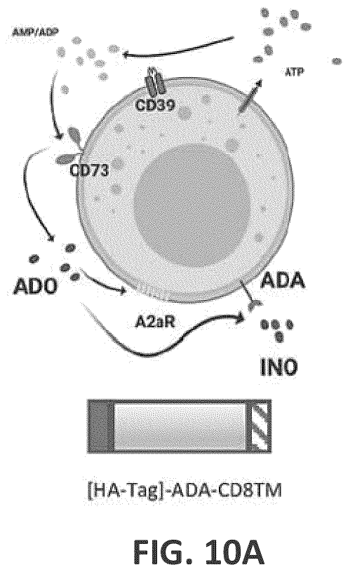

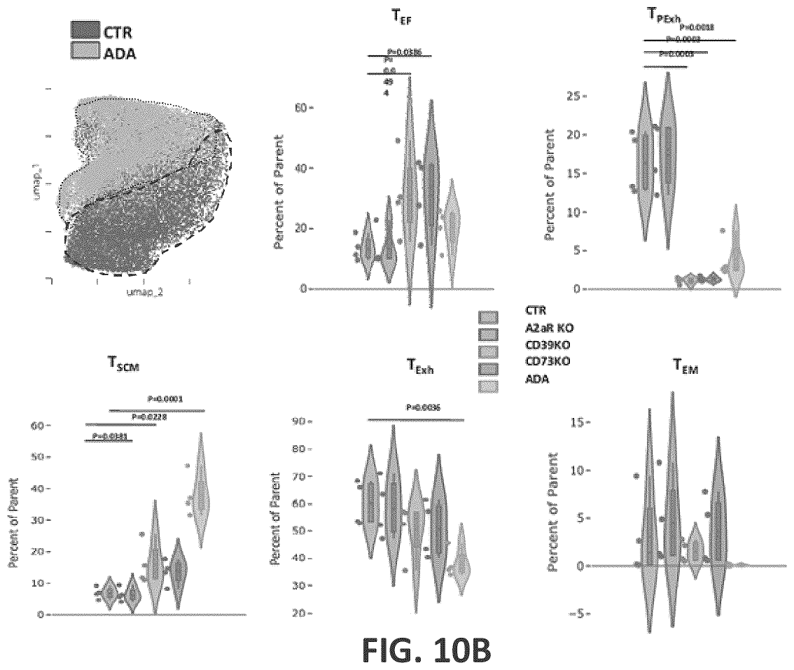

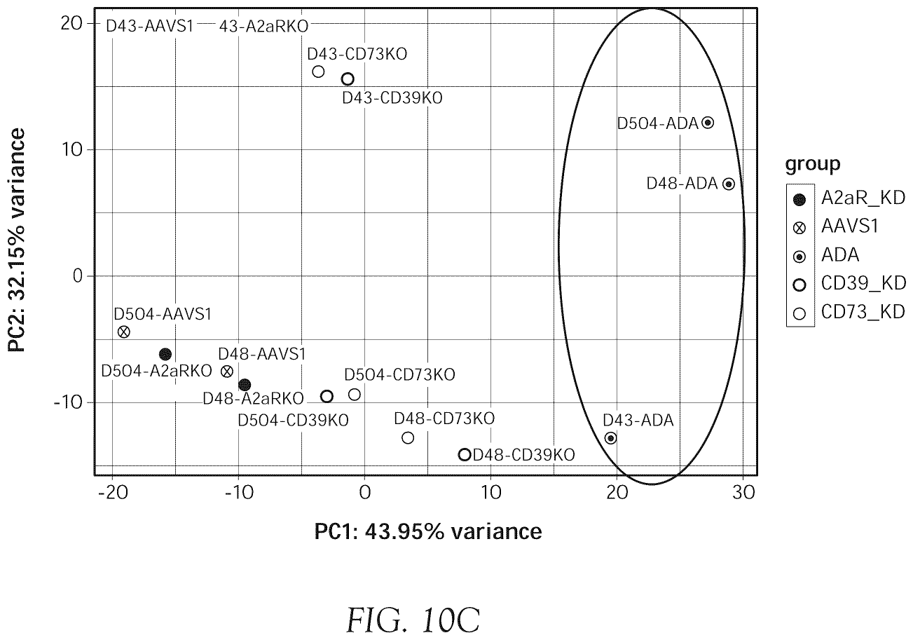

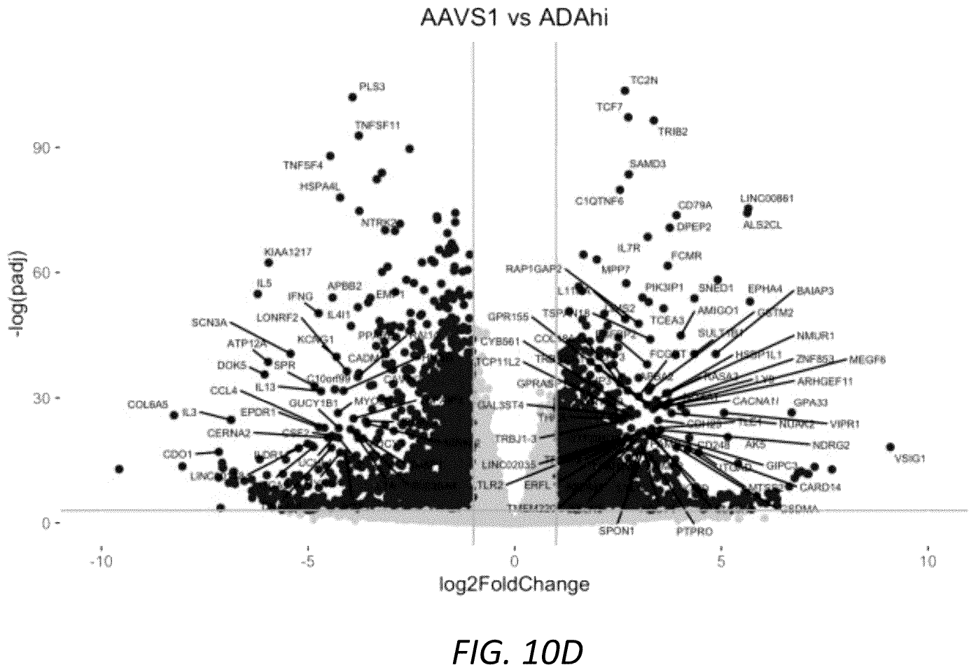

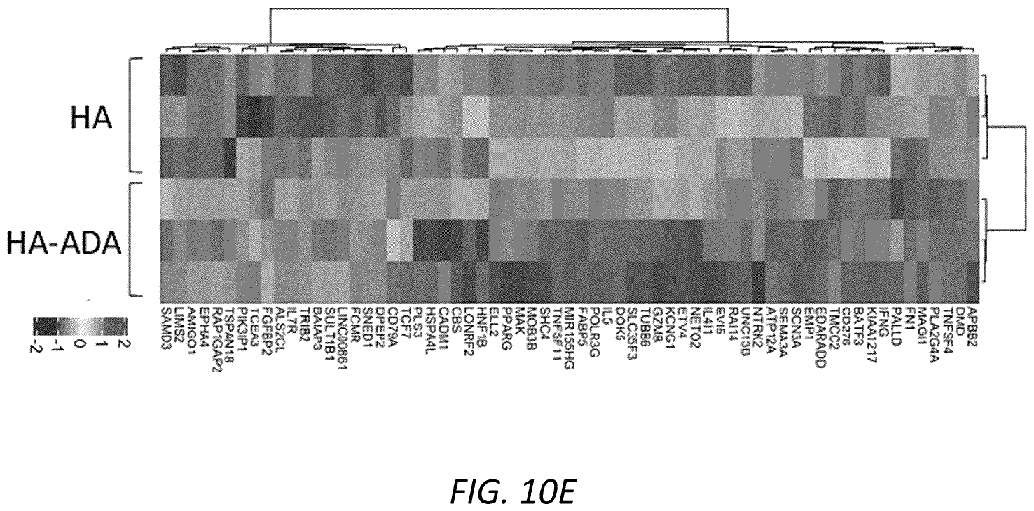

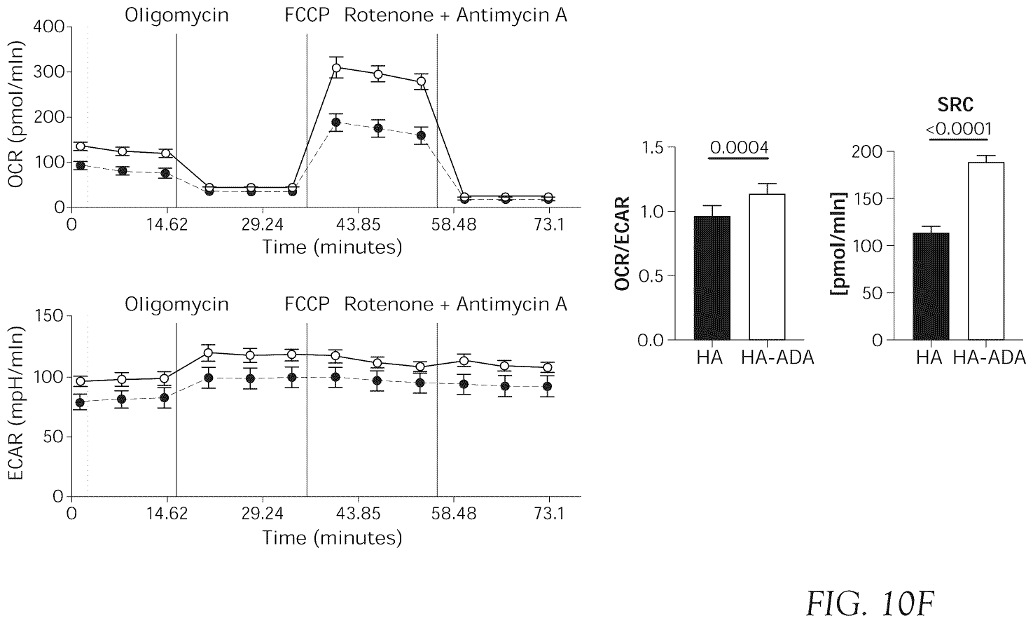

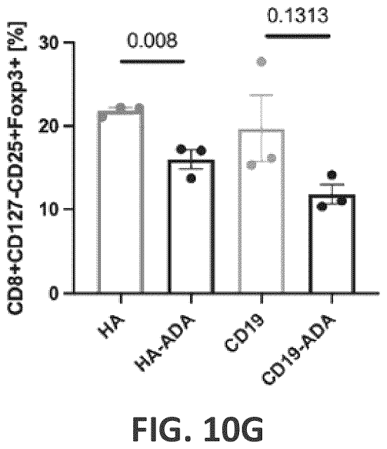

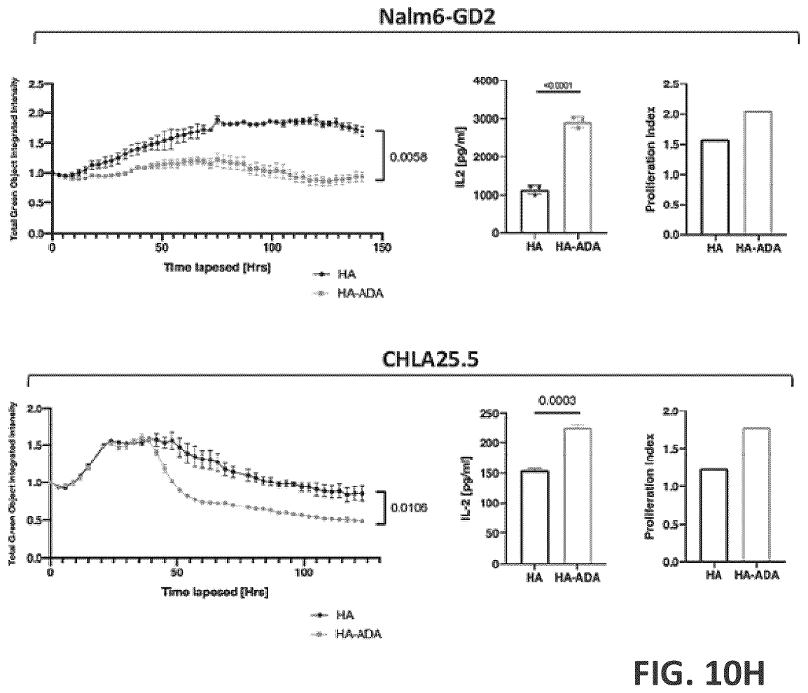

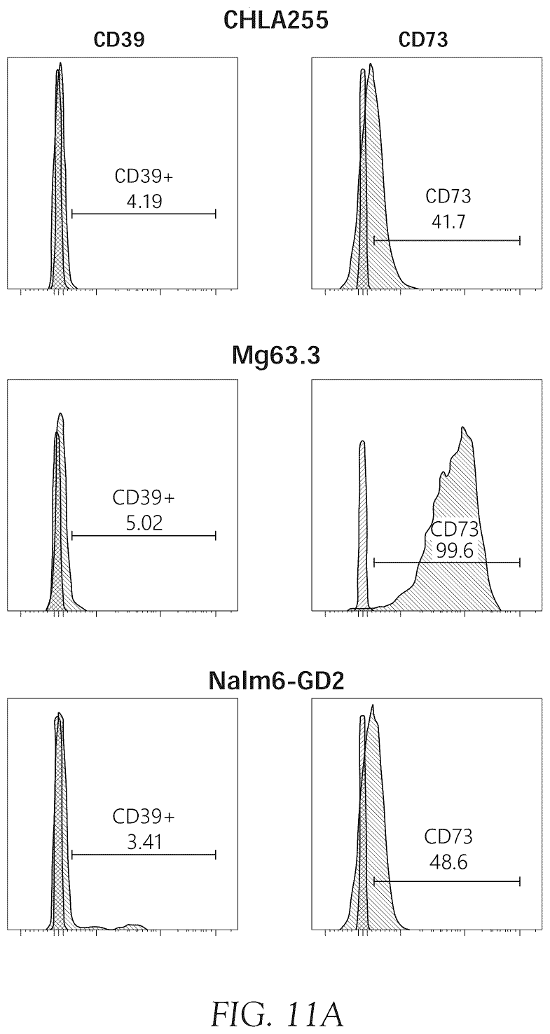

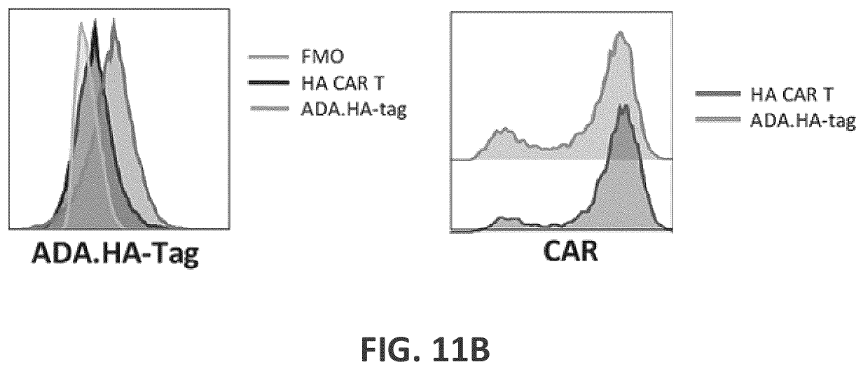

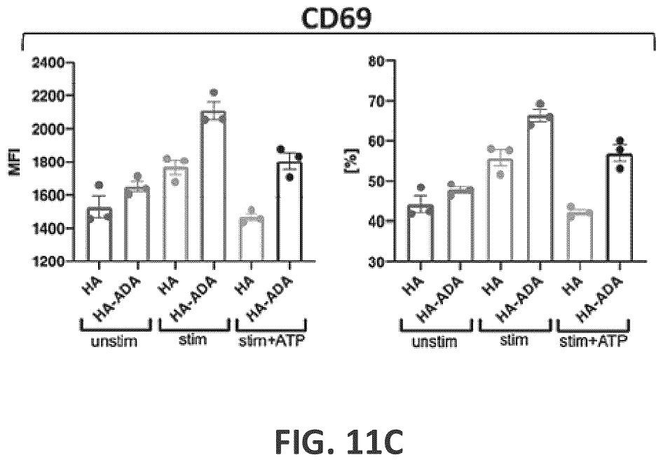

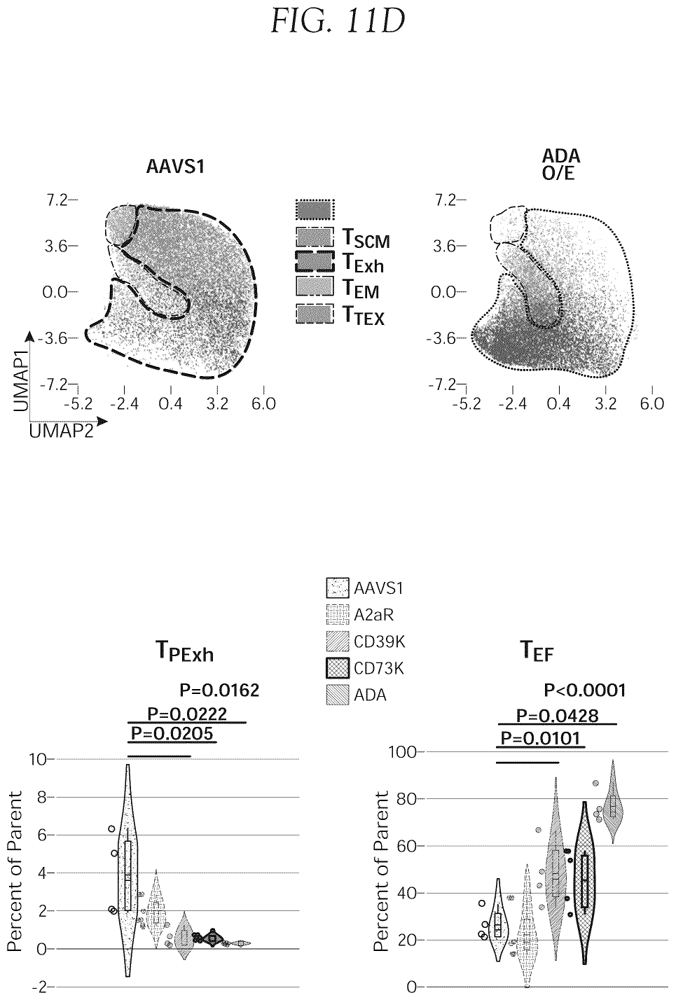

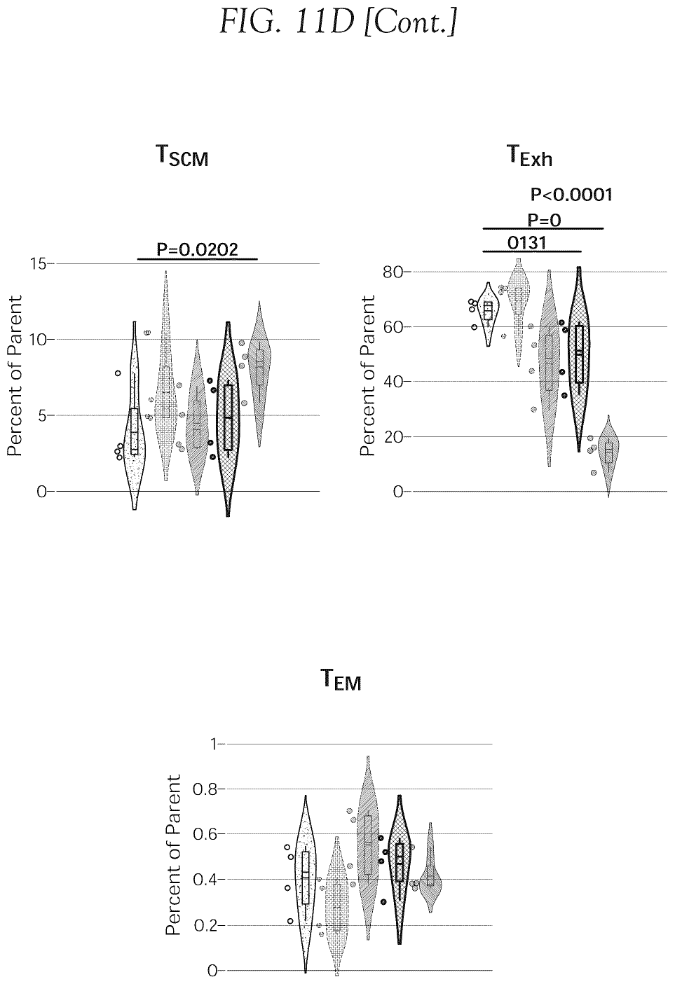

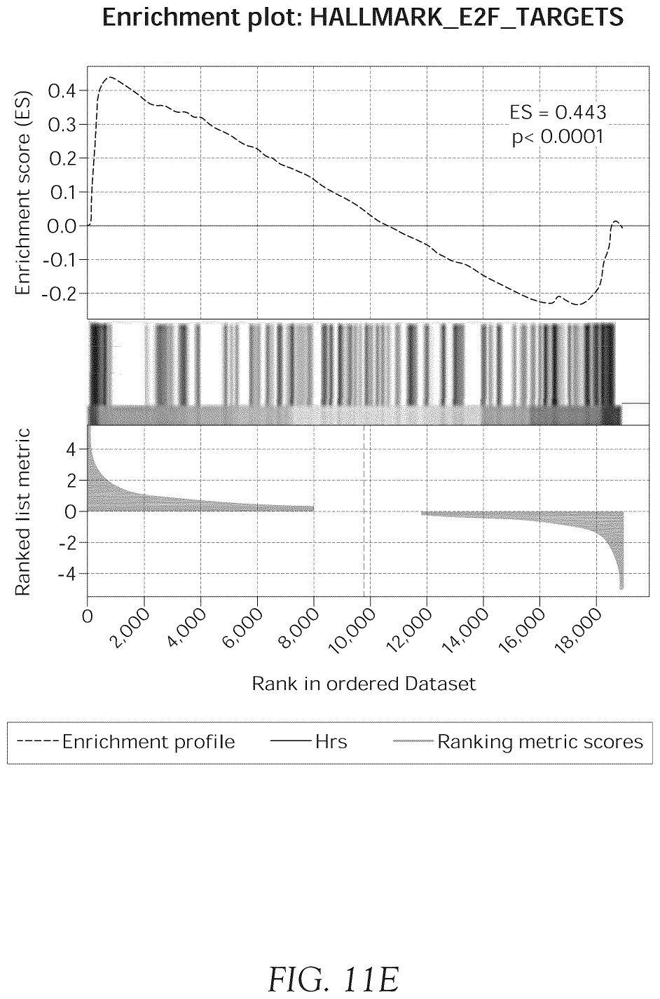

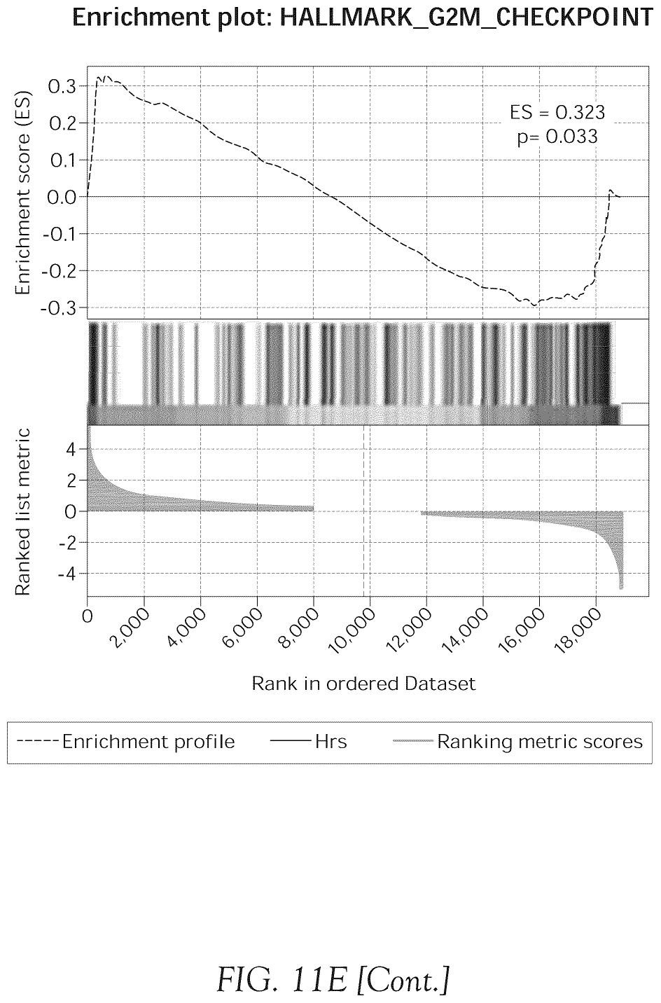

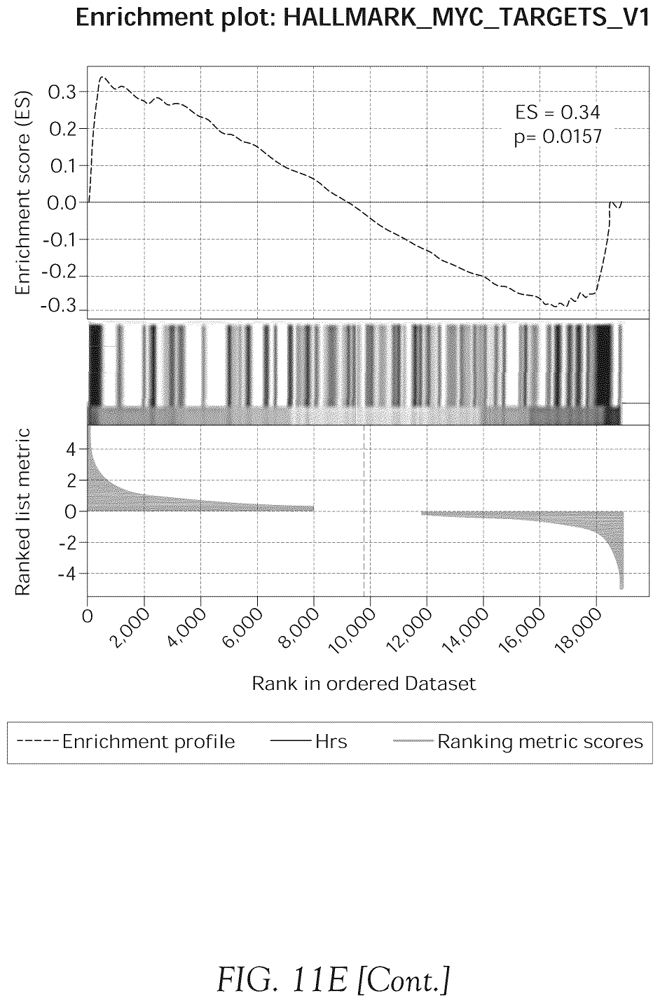

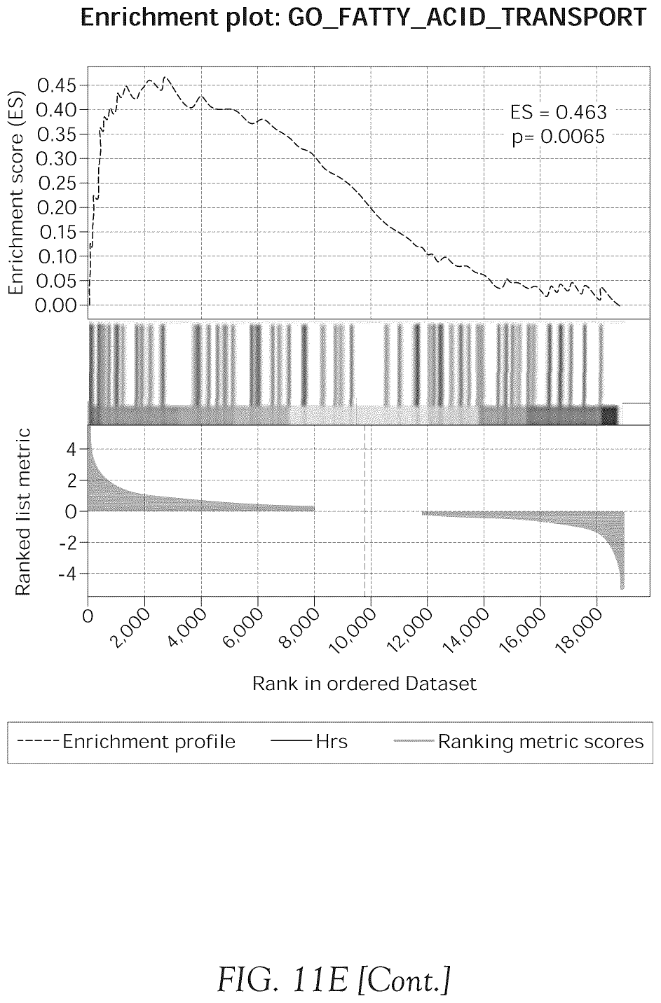

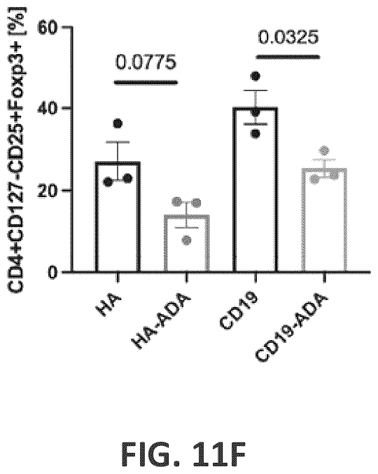

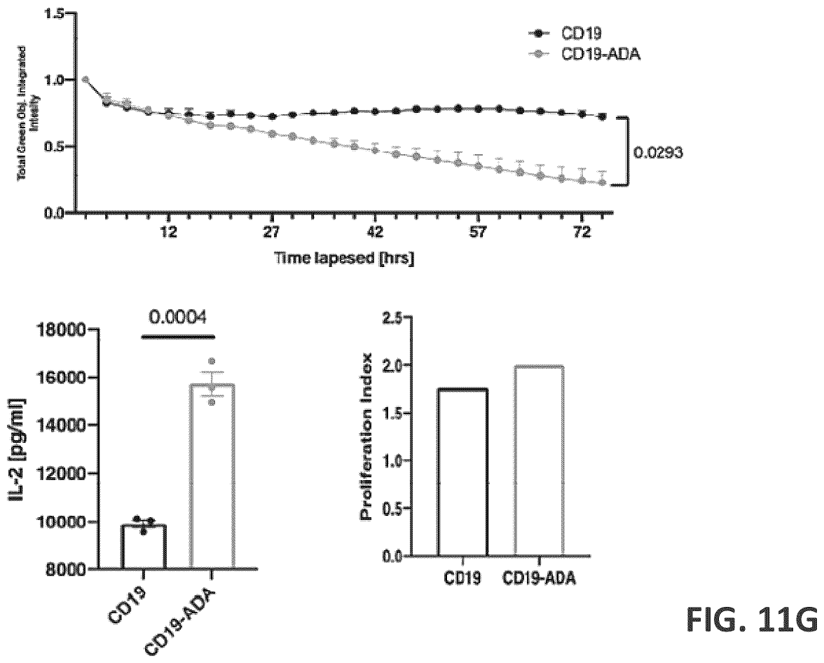

As described in greater detail below, overexpression of adenosine deaminase can improve CAR T cells phenotype and effector function. In the context of the tumor microenvironment, production of adenosine is not only regulated by CD39 and CD73 present on CAR T cells, but also expressed on the surface of cancer-associated fibroblasts, stromal, or directly on the tumor cells. While knocking out CD39 or CD73 may improve cytokine secretion in vitro, it may not be a successful approach in vivo. Similarly, although A2a receptor exhibits the highest affinity for adenosine, it is not the only adenosine receptor expressed by T cells. Therefore, an alternative approach to diminish the suppressive effect of adenosine on CAR T cells is to overexpress the enzyme responsible for metabolizing adenosine into inosine, the adenosine deaminase (ADA). In some embodiments of the disclosure, to ensure that overexpressed ADA can anchor on the surface of a T cell, it can be fused to a polypeptide transmembrane domain, for example a transmembrane domain of e.g. CD8. As described in greater detail below, transmembrane-bound ADA overexpression significantly and robustly increase fitness and function of CAR T cells, both exhausted and non-exhausted, by shifting their phenotype towards memory-like cells (see, e.g., Examples 8 and 9). In particular, exhausted CAR T cells engineered to overexpress transmembrane-bound ADA had increased spare respiratory capacity (SRC), a characteristic of memory T cells. For example, genes associated with memory phenotype and persistence, such as TCF7, IL7R were found to be upregulated in ADA-overexpressing HA CAR T cells (see, e.g., FIG. 10E). There was an upregulation of many genes involved in cell proliferation, cMYC regulated pathways, and fatty acid metabolism (see, e.g., FIG. 11E). In contrast, genes associated with effector function such as granzyme B, IL-3, IL-5, TNFSF4 (OX40), or TNFSF11 (RANKL) were downregulated. In addition, transmembrane-bound ADA overexpression significantly decreases Foxp3 frequency in exhausted HA and non-exhausted CD19 CD8 and CD4 CAR T cells (see, e.g., FIGS. 10G and 11F). Decreased percent of Tregs in ADA+ CAR T cells translated into increased effector function against tumor lines expressing different surface antigen density and proliferation (see, e.g., FIGS. 10H and 11G).

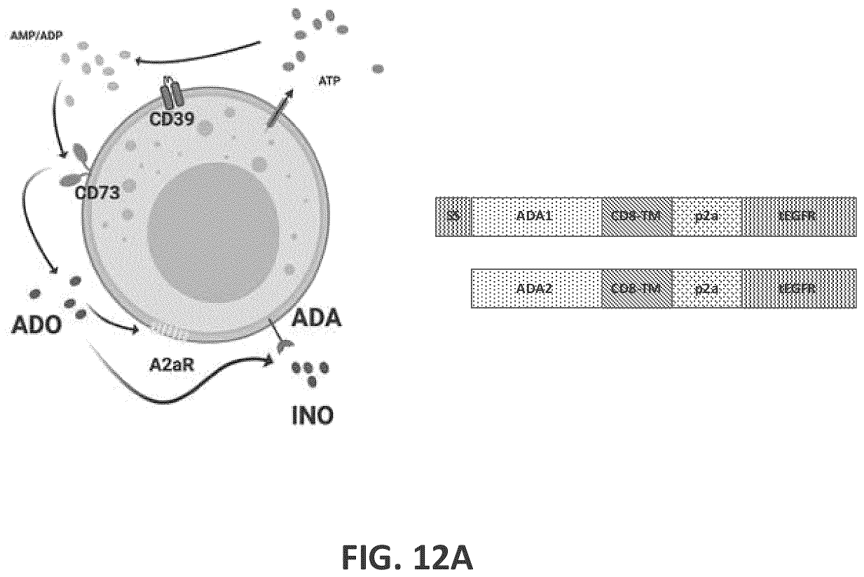

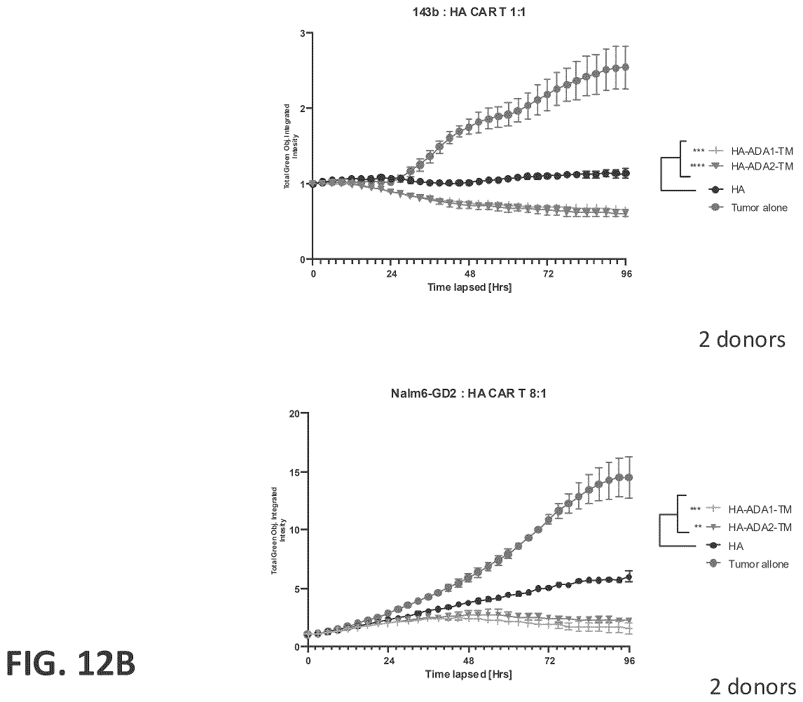

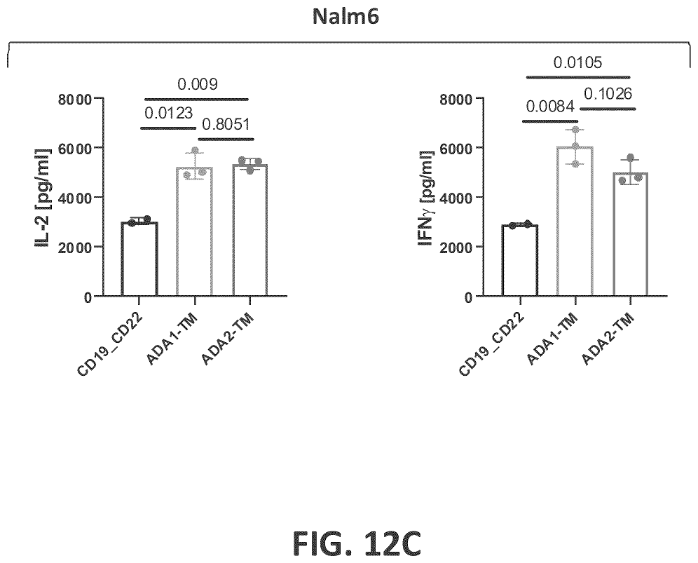

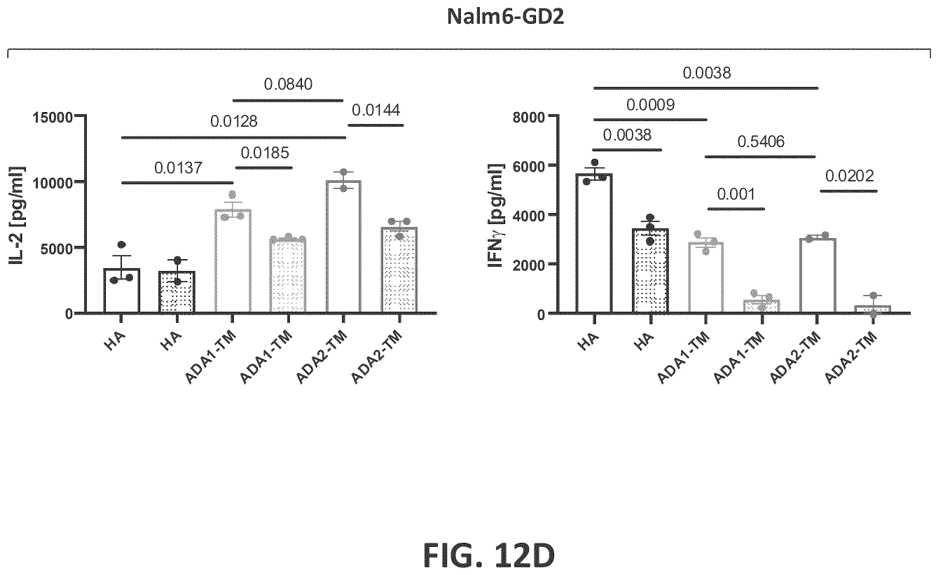

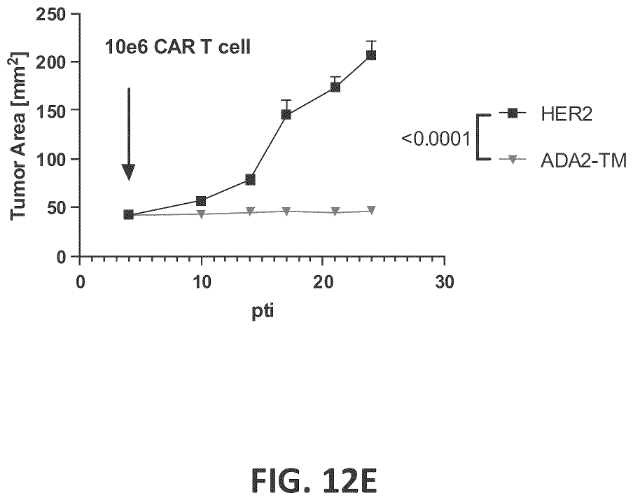

Furthermore, the experimental data described herein provides the evidence that overexpression of transmembrane-bound ADA1 or transmembrane-bound ADA2 significantly improves effector function of exhausted and non-exhausted CAR T cells in vitro and as well as in vivo. In particular, CAR T cells engineered to overexpress either one of membrane-bound ADA1 and membrane-bound ADA2 resulted in an increased tumor killing compared to the control CAR T cells expressing HA (see e.g., FIG. 12B). both CAR T cells overexpressing either HA-ADA1-TM or HA-ADA2-TM produced more IL-2 and IFNγ as compared to control group and this increase was abrogated in the presence of EHNA (see e.g., FIG. 12D). These results indicate that elevated cytokine secretion by CAR T cells was mediated by enzymatic activity of transmembrane-bound ADA.

In one aspect, provided herein is a chimeric polypeptide comprising a first polypeptide module having adenosine deaminase activity and a second polypeptide module capable of anchoring (e.g., attaching, tethering, or immobilizing) the adenosine deaminase activity to a surface of a T cell.

Non-limiting exemplary embodiments of the chimeric polypeptides according to the present disclosure include one or more of the following features. In some embodiments, the first polypeptide module is operably linked to the second polypeptide module. In some embodiments, the first polypeptide module having a human adenosine deaminase activity. In some embodiments, the adenosine deaminase activity is of ADA1, ADA2, or a functional variant of any thereof. In some embodiments, the first polypeptide module having a human ADA1 activity or a functional variant thereof. In some embodiments, the first polypeptide module having a human ADA2 activity or a functional variant thereof. In some embodiments, the first polypeptide module comprises an amino acid sequence having at least 80% sequence identity, for example, at least 80%, at least 85%, at least 90%, at least 95%, at least 100% sequence identity to SEQ ID NO: 7. In some embodiments, the first polypeptide module comprises an amino acid sequence having at least 95%, at least 96%, at least 97%, at least 98%, at least 99%, at least 100% sequence identity to the sequence of SEQ ID NO: 7. In some embodiments, the first polypeptide module comprises a amino acid sequence having 100% sequence identity to SEQ ID NO: 7. In some embodiments, the first polypeptide module comprises an amino acid sequence having 100% sequence identity to SEQ ID NO: 7, wherein one, two, three, four, or five of the amino acid residues in SEQ ID NO: 7 is/are substituted by a different amino acid residue.

In some embodiments, the first polypeptide module comprises an amino acid sequence having at least 80% sequence identity, for example, at least 80%, at least 85%, at least 90%, at least 95%, at least 100% sequence identity to SEQ ID NO: 8. In some embodiments, the first polypeptide module comprises an amino acid sequence having at least 95%, at least 96%, at least 97%, at least 98%, at least 99%, at least 100% sequence identity to the sequence of SEQ ID NO: 8. In some embodiments, the first polypeptide module comprises a amino acid sequence having 100% sequence identity to SEQ ID NO: 8. In some embodiments, the first polypeptide module comprises an amino acid sequence having 100% sequence identity to SEQ ID NO: 8, wherein one, two, three, four, or five of the amino acid residues in SEQ ID NO: 8 is/are substituted by a different amino acid residue.

As described above, in some embodiments of the disclosure, the second polypeptide module of the chimeric polypeptide includes a polypeptide transmembrane domain. Non-limiting examples of transmembrane domains suitable for the compositions and methods of the disclosure include those derived from CD8a, CD4, CD28, CD80, ICOS, CTLA4, PD1, PD-L1, BTLA, HVEM, CD27, 4-1BB, 4-1BBL, OX40, OX40L, DR3, GITR, CD30, SLAM, CD2, 2B4, TIM1, TIM2, TIM3, TIGIT, CD226, CD160, LAG3, LAIR1, B7-1, B7-H1, and B7-H transmembrane domain. In some embodiments, the polypeptide transmembrane domain is a CD8 transmembrane domain or a functional variant thereof. In some embodiments, the CD8 transmembrane domain comprises an amino acid sequence having at least 80% sequence identity, for example, at least 80%, at least 85%, at least 90%, at least 95%, at least 100% sequence identity to SEQ ID NO: 9.

In some embodiments, the CD8 transmembrane domain comprises an amino acid sequence having at least 95%, at least 96%, at least 97%, at least 98%, at least 99%, at least 100% sequence identity to the sequence of SEQ ID NO: 7. In some embodiments, the CD8 transmembrane domain comprises an amino acid sequence having 100% sequence identity to SEQ ID NO: 9. In some embodiments, the CD8 transmembrane domain comprises an amino acid sequence having 100% sequence identity to SEQ ID NO: 9, wherein one, two, three, four, or five of the amino acid residues in SEQ ID NO: 9 is/are substituted by a different amino acid residue. In some embodiments, the first polypeptide module is operably linked to the second polypeptide module.

Designation of the amino acid sequence of the chimeric polypeptide that includes a polypeptide module having adenosine deaminase activity as the “first” polypeptide module and the amino acid sequence of the chimeric polypeptide that includes a polypeptide module including a polypeptide transmembrane domain as the “second” polypeptide module is not intended to imply any particular structural arrangement of the “first” and “second” amino acid sequences within the chimeric polypeptide. By way of non-limiting example, in some embodiments of the disclosure, the chimeric polypeptide may include an N-terminal polypeptide module having adenosine deaminase activity and a C-terminal polypeptide module including a polypeptide transmembrane domain. In other embodiments, the chimeric polypeptide may include an N-terminal polypeptide module including a polypeptide transmembrane domain and a C-terminal polypeptide module having adenosine deaminase activity. In addition or alternatively, the chimeric polypeptide may include more than one polypeptide module having adenosine deaminase activity, and/or more than one polypeptide module including a polypeptide transmembrane domain. Accordingly, in some embodiments, a first amino acid sequence of the chimeric polypeptide includes at least two, three, four, five, six, seven, eight, nine, or ten polypeptide modules each having adenosine deaminase activity. In some embodiments, the at least two, three, four, five, six, seven, eight, nine, or ten polypeptide modules of a second amino acid sequence are each including a polypeptide transmembrane domain.

In some embodiments, a first amino acid sequence of the chimeric polypeptide is operably linked to a second amino acid sequence via a linker. There is no particular limitation on the linkers that can be used in the multivalent polypeptides described herein. In some embodiments, the linker is a synthetic compound linker such as, for example, a chemical cross-linking agent. Non-limiting examples of suitable cross-linking agents that are commercially available include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES). Other examples of alterative structures and linkages suitable for the multivalent polypeptides and multivalent antibodies of the disclosure include those described in Spiess et al., Mol. Immunol. 67:95-106, 2015.

In some embodiments, a first amino acid sequence of a chimeric polypeptide disclosed herein is operably linked to a second amino acid sequence via a linker polypeptide sequence (peptidal linkage). In principle, there are no particular limitations to the length and/or amino acid composition of the linker polypeptide sequence. In some embodiments, the polypeptide linker includes a single-chain peptide comprising about one to 100 amino acid residues (e.g., 2, 3, 4, 5, 6, 7, 8, 9, 10, 11, 12, 13, 14, 15, 16, 17, 18, 19, 20, etc. amino acid residues). In some embodiments, the linker polypeptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker polypeptide sequence includes about 1 to 10, about 5 to 15, about 10 to 20, about 15 to 25, about 20 to 40, about 30 to 50, about 40 to 60, about 50 to 70 amino acid residues. In some embodiments, the linker polypeptide sequence includes about 40 to 70, about 50 to 80, about 60 to 80, about 70 to 90, or about 80 to 100 amino acid residues. In some embodiments, the linker polypeptide sequence includes about 1 to 10, about 5 to 15, about 10 to 20, about 15 to 25 amino acid residues.

### Nucleic Acids

In an aspect, provided herein is isolated nucleic acids encoding the chimeric polypeptides described herein, expression cassettes encoding the chimeric polypeptides described herein, and expression vectors containing the isolated nucleic acids encoding the chimeric polypeptides described herein. In some embodiments, isolated nucleic acids can be operably linked to regulator sequences which facilitate expression of the chimeric polypeptides in a host cell.

The terms “nucleic acid” and “polynucleotide” can be used interchangeably herein, and refer to both RNA and DNA molecules, including nucleic acids comprising cDNA, genomic DNA, synthetic DNA, and DNA or RNA molecules containing nucleic acid analogs. A nucleic acid can be double-stranded or single-stranded (e.g., a sense strand or an antisense strand). A nucleic acid can contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR § 1.822 is used herein.

The nucleic acids of the present disclosure can be nucleic acids of any length, including nucleic acids that are generally between about generally between about 0.5 Kb and about 20 Kb, for example between about 0.5 Kb and about 20 Kb, between about 1 Kb and about 15 Kb, between about 2 Kb and about 10 Kb, or between about 5 Kb and about 25 Kb, for example between about 10 Kb to 15 Kb, between about 15 Kb and about 20 Kb, between about 5 Kb and about 20 Kb, about 5 Kb and about 10 Kb, or about 10 Kb and about 25 Kb.

In some embodiments disclosed herein, the nucleic acids of the disclosure include a nucleotide sequence encoding a chimeric polypeptide which include (i) first polypeptide module having adenosine deaminase activity, and (ii) a second polypeptide module capable of anchoring (e.g., attaching, tethering, or immobilizing) the adenosine deaminase activity to a surface of a T cell.

In some embodiments, the nucleic acids include a nucleotide sequence encoding the chimeric polypeptide that includes an amino acid sequence having at least 80%, at least 85%, at least 90%, at least 95%, at least 96%, at least 97%, at least 98%, at least 99%, or 100% sequence identity to the amino acid sequence of the chimeric polypeptide as disclosed herein or a functional fragment thereof.

Nucleic acid sequences having a high degree of sequence identity (e.g., at least 80%, at least 85%, at least 90%, at least 95%, at least 96%, at least 97%, at least 98%, at least 99%, or 100%) to a sequence of a modified genome or RNA replicon of an alphavirus species of interest can be identified and/or isolated by using the sequences identified herein (e.g., SEQ ID NOs: 7-9) or any others as they are known in the art, by genome sequence analysis, hybridization, and/or PCR with degenerate primers or gene-specific primers from sequences identified in the alphavirus species genome.

In some embodiments, the nucleic acids as disclosed herein can be incorporated into an expression cassette or an expression vector. Accordingly, some embodiments disclosed herein relate to vectors or expression cassettes including the nucleic acids as disclosed herein. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette can be inserted into a vector for targeting to a desired host cell and/or into an individual. As such, in some embodiments, an expression cassette of the disclosure includes a coding sequence for the chimeric polypeptide as disclosed herein, which is operably linked to expression control elements, such as a promoter, and optionally, any or a combination of other nucleic acid sequences that affect the transcription or translation of the coding sequence.

In some embodiments, the nucleic acids of the disclosure can be incorporated into an expression vector. It will be understood by one skilled in the art that the term “vector” generally refers to a recombinant polynucleotide construct designed for transfer between host cells, and that can be used for the purpose of transformation, e.g., the introduction of heterologous DNA into a host cell. As such, in some embodiments, the vector can be a plasmid, phage, or cosmid, into which another DNA segment can be inserted so as to bring about the replication of the inserted segment. In some embodiments, the expression vector can be an integrating vector. Accordingly, also provided herein are vectors, plasmids or viruses containing one or more of the nucleic acids encoding any of the chimeric polypeptides disclosed herein. The nucleic acid described above can be contained within a vector that is capable of directing their expression in, for example, a cell that has been transduced with the vector. Suitable vectors for use in eukaryotic and prokaryotic cells are known in the art and are commercially available or readily prepared by a skilled artisan. Additional vectors can also be found, for example, in Ausubel, F. M., et al., Current Protocols in Molecular Biology, (Current Protocol, 1994) and Sambrook et al., “Molecular Cloning: A Laboratory Manual,” 2nd ED. (1989).

It should be understood that not all vectors and expression control sequences will function equally well to express the DNA sequences described herein. Neither will all hosts function equally well with the same expression system. However, one of skill in the art can make a selection among these vectors, expression control sequences and hosts without undue experimentation. For example, in selecting a vector, the host must be considered because the vector must replicate in it. The vector's copy number, the ability to control that copy number, and the expression of any other proteins encoded by the vector, such as antibiotic markers, should also be considered. For example, vectors that can be used include those that allow the DNA encoding the multivalent polypeptides and multivalent antibodies of the present disclosure to be amplified in copy number. Such amplifiable vectors are known in the art. They include, for example, vectors able to be amplified by DHFR amplification (see, e.g., Kaufman, U.S. Pat. No. 4,470,461) or glutamine synthetase (“GS”) amplification (see, e.g., U.S. Pat. No. 5,122,464 and European published application EP 338,841).

Accordingly, in some embodiments, the chimeric polypeptides of the present disclosure can be expressed from vectors, generally expression vectors. The vectors are useful for autonomous replication in a host cell or can be integrated into the genome of a host cell upon introduction into the host cell, and thereby are replicated along with the host genome (e.g., non-episomal mammalian vectors). Expression vectors are capable of directing the expression of coding sequences to which they are operably linked. In general, expression vectors of utility in recombinant DNA techniques are often in the form of plasmids (vectors). However, other forms of expression vectors, such as viral vectors (e.g., replication defective retroviruses, adenoviruses, and adeno-associated viruses) are also included.

Exemplary recombinant expression vectors can include one or more regulatory sequences, selected on the basis of the host cells to be used for expression, operably linked to the nucleic acid sequence to be expressed.

Suitable methods for transforming or transfecting host cells can be found in Sambrook et al. (1989) Molecular Cloning: A Laboratory Manual (2nd ed., Cold Spring Harbor Laboratory Press, Plainview, N.Y.) and other standard molecular biology laboratory manuals.

The nucleic acid sequences encoding the chimeric polypeptides of the present disclosure can be optimized for expression in the host cell of interest. For example, the G-C content of the sequence can be adjusted to levels average for a given cellular host, as calculated by reference to known genes expressed in the host cell. Methods for codon optimization are known in the art. Codon usages within the coding sequence of the chimeric polypeptides disclosed herein can be optimized to enhance expression in the host cell, such that about 1%, about 5%, about 10%, about 25%, about 50%, about 75%, or up to 100% of the codons within the coding sequence have been optimized for expression in a host cell. In these instances, the expression of the codon optimized polypeptides may be enhanced by at least about 20%, about 50%, about 60%, about 70%, about 80%, about 90%, or about 100% as compared to a reference polypeptide, e.g., the original polypeptide that has not been codon optimized.

In selecting an expression control sequence, a variety of factors should also be considered. These include, for example, the relative strength of the sequence, its controllability, and its compatibility with the actual DNA sequence encoding the subject chimeric polypeptides, particularly as regards potential secondary structures. Hosts should be selected by consideration of their compatibility with the chosen vector, the toxicity of the product coded for by the DNA sequences of this disclosure, their secretion characteristics, their ability to fold the polypeptides correctly, their fermentation or culture requirements, and the ease of purification of the products coded for by the DNA sequences.

Viral vectors that can be used in the disclosure include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

The nucleic acids are not limited to sequences that encode the chimeric polypeptides; some or all of the non-coding sequences that lie upstream or downstream from a coding sequence can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

Exemplary nucleic acids of the present disclosure can include fragments not found as such in the natural state. Thus, this disclosure encompasses recombinant nucleic acid molecules, such as those in which a nucleic acid sequence (for example, a sequence encoding the chimeric polypeptide of the disclosure) is incorporated into a vector (e.g., a plasmid or viral vector) or into the genome of a host cell (e.g., T cell).

### Engineered T Cells

The nucleic acids encoding the chimeric polypeptides of the present disclosure can be introduced into a host cell, such as, for example, a human T lymphocyte, to produce an engineered T cell containing the nucleic acids. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

In some embodiments, host cells (e.g., T cells) can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. Host cells (e.g., T cells) can be either untransformed cells or cells that have already been transfected with at least one nucleic acid molecule.

In some embodiments, the host cell is an animal cell. In some embodiments, the animal cell is a mammalian cell. In some embodiments, the animal cell is a human cell. In some embodiments, the cell is a non-human primate cell.

In some embodiments, the host cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell (T_{H}), a cytotoxic T cell (T_{CTL}), or other T cell.

In some embodiments, the immune system cell is a T lymphocyte. In some embodiments, the cell can be obtained by leukapheresis performed on a sample obtained from a subject. In some embodiments, the subject is a human patient or a subject who has, is at risk of having, or is suspected of having a disease of interest (e.g., cancer) and/or one or more symptoms of the disease.

Accordingly, in an aspect, provided herein is an engineered T cell produced by any of the methods provided herein. In some embodiments, the T cell is a CD8+ T cytotoxic lymphocyte cell or a CD4+ T helper lymphocyte cell. In some embodiments, the CD8+ T cytotoxic lymphocyte cell is selected from the group consisting of naïve CD8+ T cells, central memory CD8+ T cells, effector memory CD8+ T cells, effector CD8+ T cells, CD8+ stem memory T cells, bulk CD8+ T cells. In some embodiments, the CD4+ T helper lymphocyte cell is selected from the group consisting of naïve CD4+ T cells, central memory CD4+ T cells, effector memory CD4+ T cells, effector CD4+ T cells, CD4+ stem memory T cells, and bulk CD4+ T cells. In some embodiments, the T cell is an exhausted T cell. In some embodiments, the T cell is a non-exhausted T cell. In some embodiments, the T cell was obtained leukapheresis of a sample obtained from a subject.

In another aspect, provided herein are cell cultures including at least one engineered T cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one engineered cell as disclosed herein and a culture medium are also within the scope of this application. Methods and systems suitable for generating and maintaining cell cultures are known in the art.

### Pharmaceutical Compositions

The engineered T cells, chimeric polypeptides, nucleic acids encoding the chimeric polypeptides of the disclosure can be incorporated into compositions, including pharmaceutical compositions. Such compositions generally can include one or more engineered T cells, chimeric polypeptides, nucleic acids encoding the chimeric polypeptides of the disclosure and a pharmaceutically acceptable excipient, e.g., a carrier. Accordingly, in one aspect, some embodiments of the disclosure relate to pharmaceutical compositions including a pharmaceutically acceptable excipient and (a) engineered T cells of the disclosure; (b) chimeric polypeptides of the disclosure; and/or (c) nucleic acids encoding the chimeric polypeptides of the disclosure.

In some embodiments, the pharmaceutical compositions of the disclosure are formulated for the treating, ameliorating a health condition, e.g., a proliferative disease such as cancer, or for reducing or delaying the onset of the disease.

Non-limiting exemplary embodiments of the pharmaceutical compositions described herein can include one or more of the following features. In some embodiments, the composition includes a nucleic acid encoding one or more chimeric polypeptides of the disclosure, and a pharmaceutically acceptable excipient. In some embodiments, the nucleic acid is encapsulated in a viral capsid or a lipid nanoparticle. In some embodiments, the nucleic acid is incorporated into an expression cassette or an expression vector. In some embodiments, the expression vector is a viral vector. In some embodiments, the viral vector is a lentiviral vector, an adenovirus vector, an adeno-associated virus vector, or a retroviral vector.

In some embodiments, the nucleic acid can be introduced into a host immune cell, for example, a T lymphocyte to produce a recombinant immune cell containing the nucleic acid. In some embodiments, the nucleic acid can be administered into a subject in need thereof.

Introduction of the nucleic acids of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be delivered by viral or non-viral delivery vehicles known in the art. For example, the nucleic acid molecule can be stably integrated in the host genome, or can be episomally replicating, or present in the host cell as a mini-circle expression vector for transient expression. Accordingly, in some embodiments, the nucleic acid molecule is maintained and replicated in the host cell as an episomal unit. In some embodiments, the nucleic acid molecule is stably integrated into the genome of the host cell. Stable integration can be achieved using classical random genomic recombination techniques or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the host cell as a mini-circle expression vector for transient expression.

The nucleic acid molecules can be encapsulated in a viral capsid, or a liposome, or a lipid nanoparticle (LNP), or can be delivered by viral or non-viral delivery means and methods known in the art, such as electroporation. For example, introduction of nucleic acids into cells can be achieved by viral transduction. In a non-limiting example, adeno-associated virus (AAV) can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, the composition includes at least one engineered T cell of the disclosure, and a pharmaceutically acceptable excipient. In some embodiments, the at least one engineered T cell exhibits an enhanced effector function when introduced into a subject, as compared to the effector function of control T cells under similar conditions, e.g., T cells that have not been engineered. Examples of effector functions that are enhanced in the engineered T cells include, but are not limited to growth rate (proliferation), death rate, death rate type, target cell inhibition (cytotoxicity), target cell killing, target cell survival, cluster of differentiation change, macrophage activation, B cell activation, cytokine production, in vivo persistence, and increased spare respiratory capacity.

In certain embodiments, the pharmaceutical compositions in accordance with some embodiments disclosed herein include cultures of engineered T cells that can be washed, treated, combined, supplemented, or otherwise altered prior to administration to an individual in need thereof. Furthermore, administration can be at varied doses, time intervals or in multiple administrations.

In certain embodiments, the pharmaceutical compositions in accordance with some embodiments disclosed herein include engineered T cells comprising the chimeric polypeptides of the disclosure.

The pharmaceutical compositions provided herein can be in any form that allows for the composition to be administered to a subject. In some specific embodiments, the pharmaceutical compositions are suitable for human administration. As used herein, the term “pharmaceutically acceptable” means approved by a regulatory agency of the federal or a state government or listed in the U.S. Pharmacopeia or other generally recognized pharmacopeia for use in animals, and more particularly in humans. The carrier can be a diluent, adjuvant, excipient, or vehicle with which the pharmaceutical composition is administered. Saline solutions and aqueous dextrose and glycerol solutions can also be employed as liquid carriers, including injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. In some embodiments, the pharmaceutical composition is sterilely formulated for administration into an individual. In some embodiments, the individual is a human. One of ordinary skilled in the art will appreciate that the formulation should suit the mode of administration.

In some embodiments, the pharmaceutical compositions of the present disclosure are formulated to be suitable for the intended route of administration to an individual. For example, the pharmaceutical composition can be formulated to be suitable for parenteral, intraperitoneal, colorectal, intraperitoneal, and intratumoral administration. In some embodiments, the pharmaceutical composition can be formulated for intravenous, oral, intraperitoneal, intratracheal, subcutaneous, intramuscular, topical, or intratumoral administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the engineered immune cells of the disclosure can be formulated for administration to a subject using techniques known to the skilled artisan. For example, formulations comprising populations of engineered immune cells can include pharmaceutically acceptable excipient(s). Excipients included in the formulations will have different purposes depending, for example, on the engineered immune cells used and the mode of administration. Examples of generally used excipients included, without limitation: saline, buffered saline, dextrose, water-for-injection, glycerol, ethanol, and combinations thereof, stabilizing agents, solubilizing agents and surfactants, buffers and preservatives, tonicity agents, bulking agents, and lubricating agents. The formulations comprising engineered immune cells can have been prepared and cultured in the absence of non-human components, e.g., in the absence of animal serum. A formulation can include one population of engineered immune cells, or more than one, such as two, three, four, five, six or more populations of engineered immune cells.

Formulations comprising population(s) of engineered immune cells can be administered to a subject using modes and techniques known to the skilled artisan. Exemplary modes include, but are not limited to, intravenous injection. Other modes include, without limitation, intratumoral, intradermal, subcutaneous (S.C., s.q., sub-Q, Hypo), intramuscular (i.m.), intraperitoneal (i.p.), intra-arterial, intramedullary, intracardiac, intra-articular (joint), intrasynovial (joint fluid area), intracranial, intraspinal, and intrathecal (spinal fluids). Devices useful for parenteral injection of infusion of the formulations can be used to effect such administration.

### Kits

Also provided herein are kits for the practice of a method described herein. A kit can include one or more of the engineered immune cells (e.g., engineered T cells), chimeric polypeptides, nucleic acids encoding the chimeric polypeptides, and/or pharmaceutical compositions as described and provided herein. For examples, provided herein, in some embodiments, are kits that include one or more engineered T cells of the disclosure. In some embodiments, provided herein are kits that include one or more pharmaceutical compositions of the disclosure. In some embodiments, the kits of disclosure further include written instructions for making the engineered T cells, chimeric polypeptides, nucleic acids encoding the chimeric polypeptides, and/or pharmaceutical compositions of the disclosure and using the same.

In some embodiments, the kits of the disclosure further include one or more syringes (including pre-filled syringes) and/or catheters (including pre-filled syringes) used to administer one any of the provided T cells, nucleic acids, and pharmaceutical compositions to a subject in need thereof. In some embodiments, a kit can have one or more additional therapeutic agents that can be administered simultaneously or sequentially with the other kit components for a desired purpose, e.g., for modulating an activity of a cell, inhibiting a target cancer cell, or treating a health condition in a subject in need thereof.

For example, any of the above-described kits can further include one or more additional reagents, where such additional reagents can be selected from: dilution buffers; reconstitution solutions, wash buffers, control reagents, control expression vectors, negative control T-cell populations, positive control T-cell populations, reagents for ex vivo production of the T-cell populations.

In some embodiments, the components of a kit can be in separate containers. In some other embodiments, the components of a kit can be combined in a single container. For example, in some embodiments of the disclosure, the kit includes one or more of the provided immune cells, nucleic acids, and/or pharmaceutical compositions as described herein in one container (e.g., in a sterile glass or plastic vial) and a further therapeutic agent in another container (e.g., in a sterile glass or plastic vial).

In some embodiments, a kit can further include instructions for using the components of the kit to practice the methods disclosed herein. For example, the kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure can be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and intellectual property information.

In some embodiments, a kit can include further instructions for using the components of the kit to practice the methods disclosed herein. The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

### Methods of the Disclosure

As described in greater details below, one aspect of the present disclosure relates to methods of generating the presently described engineered T cells, methods of administering the engineered T cells, and methods of treating individuals of relevant health conditions, such as proliferative diseases (e.g., cancers), autoimmune diseases, and microbial infections (e.g., viral infections).

### Methods of Generating Engineered T Cells

The nucleic acids encoding the chimeric polypeptides of the present disclosure can be introduced into a host cell, such as, for example, a human T lymphocyte, to produce an engineered T cell containing the nucleic acids. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

The nucleic acids can be delivered by, for example, viral or non-viral delivery vehicles known in the art. In some embodiments, the nucleic acids can be maintained and replicated in the host cell (e.g., T cell) as an episomal unit. In some embodiments, the nucleic acids can be stably integrated into the genome of the host cell (e.g., T cell). Stable integration can be achieved using classical random genomic recombination techniques or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases). In some embodiments, the nucleic acids can present in the host cell (e.g., T cell) as a mini-circle expression vector for transient expression.

Accordingly, in an embodiments, provided herein is a method for generating an engineered T cell with enhanced effector function, the method comprising introducing into a T cell any one of chimeric polypeptides the disclosure or nucleic acid encoding the chimeric polypeptide. In some embodiments, the introduced chimeric polypeptide results in a reduced intracellular level of adenosine in the engineered T cell compared to reference T cell that does not comprise the chimeric polypeptide. In some embodiments, the introduced chimeric polypeptide results in an enhanced effector function of the engineered T cell as compared to a control T cell under similar condition, e.g., a T cell that has not been engineered to include such chimeric polypeptide. In some embodiments, the method further comprises introducing into the T cell at least one recombinant antigen-specific receptor. In some embodiments, the at least one recombinant antigen-specific receptor comprises an engineered T cell receptor (TCR) and/or an engineered chimeric antigen receptor (CAR).

### Methods of Treatment

Administration of any one of the therapeutic compositions described herein, e.g., engineered T cells, nucleic acid molecules encoding chimeric polypeptides of the disclosure, and pharmaceutical compositions, can be used to treat individuals in the treatment of relevant health conditions, such as proliferative diseases (e.g., cancers), autoimmune diseases, and microbial infections (e.g., viral infections). In some embodiments, one or more engineered T cells, nucleic acid molecules, and pharmaceutical compositions as described herein can be incorporated into therapeutic agents for use in methods of treating a subject who has, who is suspected of having, or who can be at high risk for developing one or more health conditions, such as proliferative diseases (e.g., cancers), autoimmune diseases, and chronic infections. In some embodiments, the subject is a mammalian subject. In some embodiments, the subject has or is suspected of having a proliferative disease, an autoimmune disease, or an infection. In some embodiments, the proliferative disease is a cancer. In some embodiments, the subject is a patient under the care of a physician.

Accordingly, in an aspect, provided herein is a method for preventing and/or treating a health condition in a subject in need thereof, the method comprising administering to the subject a composition comprising: (a) at least one engineered T cells of the present disclosure; and/or (b) a pharmaceutical composition of the present disclosure. In some embodiments, the health condition is a proliferative disease (e.g., cancer), an autoimmune disease, or a chronic infection. In some embodiments, the subject is a mammalian subject. In some embodiments, the mammalian subject is a human subject. In some embodiments, the engineered T cells are autologous to the subject. In some embodiments, the engineered T cells are obtained from tumor infiltrating lymphocytes (TILs) or peripheral blood mononuclear cells (PBMCs). In some embodiments, the administered composition inhibits adenosine-mediated immunosuppression in the subject. In some embodiments, the adenosine-mediated immunosuppression in the subject is inhibited by at least 10%, such as at least about 10%, at least about 20%, at least about 30%, at least about 40%, at least about 50%, at least about 60%, at least about 70%, at least about 80%, at least about 90%, at least about 2 times, about three times, about four time, about five times, about six times, about seven times, about eight times, about nine times, about 20 times, about 50 times, about 100 times, or about 200 times compared to a reference subject. In some embodiments, the reference subject is a subject that has not been administered with the same composition. In some embodiments, the reference subject is a subject that has been administered with a polypeptide having no adenosine deaminase activity, and/or having an adenosine deaminase activity that is not operably linked with a polypeptide module capable of anchoring (e.g., attaching, tethering, or immobilizing) the adenosine deaminase activity to a surface of a T cell.

In some embodiments, the administered composition confers an enhanced effector function of the engineered T cells as compared to the effector function of a control T cell under similar conditions, e.g., a T cell that has not been administered with such composition. Examples of effector functions that are enhanced in the engineered immune cells include, but are not limited to growth rate (proliferation), death rate, death rate type, target cell inhibition (cytotoxicity), cluster of differentiation change, macrophage activation, B cell activation, cytokine production, in vivo persistence, and increased spare respiratory capacity. In some embodiments, an effector function of the immune cells including the composition of the disclosure is enhanced at levels that are at least 10% higher, such as at least 10% higher than about 10%, at least higher than about 20%, at least higher than about 30%, at least higher than about 40%, at least higher than about 50%, at least higher than about 60%, at least higher than about 70%, at least higher than about 80%, at least higher than about 90%, at least higher than about 2 times, higher than about three times, higher than about four time, higher than about five times, higher than about six times, higher than about seven times, higher than about eight times, higher than about nine times, higher than about 20 times, higher than about 50 times, higher than about 100 times, or higher than about 200 times compared to a reference immune cell under similar conditions, e.g., a reference T cell that has not been engineered to include such composition. Accordingly, in some embodiments, the reference immune cell does not include a composition of the disclosure. For example, in some embodiments, the reference T cell is a T cell that has not been administered with the same composition. In some embodiments, the reference T cell is a T cell that has been administered with a polypeptide having no adenosine deaminase activity, and/or having an adenosine deaminase activity that is not operably linked with a polypeptide module capable of anchoring (e.g., attaching, tethering, or immobilizing) the adenosine deaminase activity to a surface of a T cell.

In some embodiments, the enhanced effector function comprises increased production of one or more cytokines (e.g., interferon gamma (IFNγ), tumor-necrosis factor α (TNFα), and interleukin-2 (IL-2)). In some embodiments, the administered composition confers a production of one or more cytokines that is increased by at least 10%, such as at least about 10%, at least about 20%, at least about 30%, at least about 40%, at least about 50%, at least about 60%, at least about 70%, at least about 80%, at least about 90%, at least about 2 times, about three times, about four time, about five times, about six times, about seven times, about eight times, about nine times, about 20 times, about 50 times, about 100 times, or about 200 times compared to a reference immune cell under similar conditions, e.g., reference T cell. In some embodiments, the reference T cell is a T cell that has not been administered with the same composition. In some embodiments, the reference T cell is a T cell that has been administered with a polypeptide having no adenosine deaminase activity, and/or having an adenosine deaminase activity that is not operably linked with a polypeptide module capable of anchoring (e.g., attaching, tethering, or immobilizing) the adenosine deaminase activity to a surface of a T cell.

In some embodiments, the composition is administered to the subject individually (monotherapy) or in combination with a second therapy, wherein the second therapy is selected from the group consisting of chemotherapy, radiotherapy, immunotherapy, hormonal therapy, toxin therapy, or surgery.

Non-limiting exemplary embodiments of the treatment methods described herein can include one or more of the following features. In some embodiments, the health condition is a proliferative disease or an infection. Exemplary proliferative diseases can include, without limitation, angiogenic diseases, a metastatic diseases, tumorigenic diseases, neoplastic diseases and cancers. In some embodiments, the proliferative disease is a cancer. In some embodiments, the cancer is a pediatric cancer. In some embodiments, the cancer is a pancreatic cancer, a colon cancer, an ovarian cancer, a prostate cancer, a lung cancer, mesothelioma, a breast cancer, a urothelial cancer, a liver cancer, a head and neck cancer, a sarcoma, a cervical cancer, a stomach cancer, a gastric cancer, a melanoma, a uveal melanoma, a cholangiocarcinoma, multiple myeloma, leukemia, lymphoma, and glioblastoma.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the subject inhibits metastasis of the cancer in the subject. In some embodiments, the administered composition inhibits tumor growth in the subject.

Exemplary proliferative diseases can include, without limitation, angiogenic diseases, a metastatic diseases, tumorigenic diseases, neoplastic diseases and cancers. In some embodiments, the proliferative disease is a cancer. The term “cancer” generally refers to a disease characterized by the rapid and uncontrolled growth of aberrant cells. The aberrant cells can form solid tumors or constitute a hematological malignancy. Cancer cells can spread locally or through the bloodstream and lymphatic system to other parts of the body. There are no specific limitations with respect to the cancers which can be treated by the compositions and methods of the present disclosure. Non-limiting examples of suitable cancers include ovarian cancer, renal cancer, breast cancer, prostate cancer, liver cancer, brain cancer, lymphoma, leukemia, cervical cancer, skin cancer, pancreatic cancer, colorectal cancer, lung cancer and the like.

Other cancers that can be suitable treated with the compositions and methods of the present disclosure include, but are not limited to, acute myeloblastic leukemia (AML), acute lymphoblastic leukemia (ALL), chronic myelocytic leukemia (CML), adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, colon and rectum cancer, endometrial cancer, esophagus cancer, Ewing's family of tumors (e.g. Ewing's sarcoma), eye cancer, transitional cell carcinoma, vaginal cancer, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, gallbladder cancer, gastrointestinal carcinoid tumors, gastrointestinal stromal tumors, gestational trophoblastic disease, Non-Hodgkin's lymphoma, Hodgkin's lymphoma, childhood Non-Hodgkin's lymphoma, Kaposi's sarcoma, kidney cancer, laryngeal and hypopharyngeal cancer, liver cancer, lung cancer, lung carcinoid tumors, malignant mesothelioma, multiple myeloma, myelodysplastic syndrome, rhabdomyosarcoma, salivary gland cancer, sarcomas, melanoma skin cancer, non-melanoma skin cancers, stomach cancer, testicular cancer, thymus cancer, thyroid cancer, uterine cancer (e.g., uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

Particularly suitable cancers include, but are not limited to, breast cancer, ovarian cancer, lung cancer, pancreatic cancer, mesothelioma, leukemia, lymphoma, brain cancer, prostate cancer, multiple myeloma, melanoma, bladder cancer, bone sarcomas, soft tissue sarcomas, retinoblastoma, renal tumors, neuroblastoma, and carcinomas.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the subject inhibits metastasis of the cancer in the subject. For example, in some embodiments, the composition administered to the subject can reduce metastatic nodules in the subject. In some embodiments, the administered composition inhibits tumor growth in the subject.

In some embodiments, the proliferative disease is an autoimmune disease. In some embodiments, the autoimmune disease is selected from the group consisting of rheumatoid arthritis, insulin-dependent diabetes mellitus, hemolytic anemias, rheumatic fever, thyroiditis, Crohn's disease, myasthenia gravis, glomerulonephritis, autoimmune hepatitis, multiple sclerosis, alopecia areata, psoriasis, vitiligo, dystrophic epidermolysis bullosa, systemic lupus erythematosus, moderate to severe plaque psoriasis, psoriatic arthritis, Crohn's disease, ulcerative colitis, and graft vs. host disease.

In some embodiments, the administered composition inhibits proliferation of a target cancer cell, and/or inhibits tumor growth of the cancer in the subject. For example, the target cell can be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the engineered immune cells disclosed herein, wherein the engineered immune cells inhibit the proliferation of the target cell and/or inhibit tumor growth of a target cancer in the subject compared to the proliferation of the target cell and/or tumor growth of the target cancer in subjects who have not been administered with the engineered immune cells.

Administration of the compositions described herein, e.g., engineered immune cells, nucleic acids, and pharmaceutical compositions, can be used in the stimulation of an immune response. In some embodiments, one or more of engineered immune cells, nucleic acids, and/or pharmaceutical compositions as described herein are administered to an individual after induction of remission of cancer with chemotherapy, or after autologous or allogeneic hematopoietic stem cell transplantation. In some embodiments, compositions described herein are administered to a subject in need of increasing the production of interferon gamma (IFNγ), tumor-necrosis factor alpha (TNFα), and/or interleukin-2 (IL-2) in the treated subject relative to the production of these molecules in subjects who have not been administered one of the therapeutic compositions disclosed herein.

In some embodiments, the administered composition confers an enhanced effector function of the immune cells, e.g., T cells. Examples of effector functions that are enhanced in the engineered immune cells include, but are not limited to growth rate (proliferation), death rate, death rate type, target cell inhibition (cytotoxicity), target cell killing, target cell survival, cluster of differentiation change, macrophage activation, B cell activation, cytokine production, in vivo persistence, and increased spare respiratory capacity. In some embodiments, an effector function of the immune cells including the composition of the disclosure is enhanced at levels that are at least 10% higher, such as at least 10% higher than about 10%, at least higher than about 20%, at least higher than about 30%, at least higher than about 40%, at least higher than about 50%, at least higher than about 60%, at least higher than about 70%, at least higher than about 80%, at least higher than about 90%, at least higher than about 2 times, higher than about three times, higher than about four time, higher than about five times, higher than about six times, higher than about seven times, higher than about eight times, higher than about nine times, higher than about 20 times, higher than about 50 times, higher than about 100 times, or higher than about 200 times compared to a reference immune cell under similar conditions, e.g., a reference T cell that has not been administered with such composition. In some embodiments, the reference immune cell does not include a composition of the disclosure. In some embodiments, the administered composition confers an increased cell-surface expression of ADA in the immune cells. In some embodiments, the administered composition confers a cell-surface expression of ADA that is increased by at least 10%, such as at least about 10%, at least about 20%, at least about 30%, at least about 40%, at least about 50%, at least about 60%, at least about 70%, at least about 80%, at least about 90%, at least about 2 times, about three times, about four time, about five times, about six times, about seven times, about eight times, about nine times, about 20 times, about 50 times, about 100 times, or about 200 times compared to a reference immune cell under similar conditions, e.g., a reference T cell. In some embodiments, the reference T cell is a T cell that has not been administered with the same composition. In some embodiments, the reference T cell is a T cell that has been administered with a polypeptide having no adenosine deaminase activity, and/or having an adenosine deaminase activity that is not operably linked with a polypeptide module capable of anchoring the adenosine deaminase activity to a surface of a T cell.

An effective amount of the compositions described herein, e.g., engineered T cells, nucleic acids, and/or pharmaceutical compositions, can be determined based on the intended goal, for example cancer regression. For example, where existing cancer is being treated, the amount of a composition disclosed herein to be administered can be greater than where administration of the composition is for prevention of cancer. One of ordinary skill in the art would be able to determine the amount of a composition to be administered and the frequency of administration in view of this disclosure. The quantity to be administered, both according to number of treatments and dose, also depends on the individual to be treated, the state of the individual, and the protection desired. Precise amounts of the composition also depend on the judgment of the practitioner and are peculiar to each subject. Frequency of administration could range from 1-2 days, to 2-6 hours, to 6-10 hours, to 1-2 weeks or longer depending on the judgment of the practitioner.

Determination of the amount of compositions to be administered will be made by one of skill in the art, and will in part be dependent on the extent and severity of cancer, and whether the engineered immune cells, e.g., T cells, are being administered for treatment of existing cancer or prevention of cancer. For example, longer intervals between administration and lower amounts of compositions can be employed where the goal is prevention. For instance, amounts of compositions administered per dose can be 50% of the dose administered in treatment of active disease, and administration can be at weekly intervals. One of ordinary skill in the art, in light of this disclosure, would be able to determine an effective amount of compositions and frequency of administration. This determination would, in part, be dependent on the particular clinical circumstances that are present (e.g., type of cancer, severity of cancer).

In some embodiments, it can be desirable to provide a continuous supply of a composition disclosed herein to the subject to be treated, e.g., a patient. In some embodiments, continuous perfusion of the region of interest (such as a tumor) can be suitable. The time period for perfusion would be selected by the clinician for the particular subject and situation, but times could range from about 1-2 hours, to 2-6 hours, to about 6-10 hours, to about 10-24 hours, to about 1-2 days, to about 1-2 weeks or longer. Generally, the dose of the composition via continuous perfusion will be equivalent to that given by single or multiple injections, adjusted for the period of time over which the doses are administered.

In some embodiments, administration is by intravenous infusion. An effective amount of the engineered T cells, nucleic acids, and/or pharmaceutical compositions disclosed herein can be determined based on the intended goal, for example tumor regression. For example, where existing cancer is being treated, the number of cells to be administered can be greater than where administration of the engineered immune cells, e.g., T cells, disclosed herein is for prevention of cancer. One of ordinary skill in the art would be able to determine the number of cells to be administered and the frequency of administration in view of this disclosure. The quantity to be administered, both according to number of treatments and dose, also depends on the individual to be treated, the state of the individual, and the protection desired. Precise amounts of the therapeutic composition also depend on the judgment of the practitioner and are peculiar to each individual. Frequency of administration could range from 1-2 days, to 2-6 hours, to 6-10 hours, to 1-2 weeks or longer depending on the judgment of the practitioner. Generally, the dose of the therapeutic composition via continuous perfusion will be equivalent to that given by single or multiple injections, adjusted for the period of time over which the doses are administered.

### Administration of Engineered Immune Cells to a Subject.

In some embodiments, the methods of the disclosure involve administering an effective amount or number of the engineered immune cells, e.g., engineered T cells, provided herein to a subject in need thereof. This administering step can be accomplished using any method of implantation delivery in the art. For example, the engineered immune cells, e.g., engineered T cells, can be infused directly in the subject's bloodstream or otherwise administered to the subject.

In some embodiments, the methods disclosed herein include administering, which term is used interchangeably with the terms “introducing,” implanting,” and “transplanting,” engineered immune cells, e.g., engineered T cells, into an individual, by a method or route that results in at least partial localization of the introduced cells at a desired site such that a desired effect(s) is/are produced. The engineered immune cells, e.g., engineered T cells, or their differentiated progeny can be administered by any appropriate route that results in delivery to a desired location in the individual where at least a portion of the administered cells or components of the cells remain viable. The period of viability of the cells after administration to a subject can be as short as a few hours, e.g., twenty-four hours, to a few days, to as long as several years, or even the lifetime of the individual, e.g., long-term engraftment.

When provided prophylactically, the engineered immune cells, e.g., engineered T cells, described herein can be administered to a subject in advance of any symptom of a disease or health condition to be treated. Accordingly, in some embodiments the prophylactic administration of an engineered T cell population prevents the occurrence of symptoms of the disease or health condition.

When provided therapeutically in some embodiments, engineered immune cells are provided at (or after) the onset of a symptom or indication of a disease or health condition, e.g., upon the onset of disease or health condition.

For use in the various embodiments described herein, an effective amount of engineered immune cells, e.g., T cells, as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof.

In some embodiments, the engineered immune cells, e.g., T cells, are non-autologous to the subject in need of treatment. In some embodiments, the adoptive cell therapy is an allogeneic adoptive cell therapy. For example, in some embodiments, the engineered immune cells, e.g., T cells, are allogeneic to the subject in need of treatment. In an allogeneic adoptive cell therapy, the engineered immune cells, e.g., T cells, are not derived from the individual receiving the adoptive cell therapy. Allogeneic cell therapy generally refers to a therapy whereby the individual (donor) who provides the immune cells is a different individual (of the same species) than the individual receiving the cell therapy. For example, a population of engineered immune cells being administered to an individual is derived from one more unrelated donors, or from one or more non-identical siblings. Accordingly, the engineered immune cells can be derived from one or more donors or can be obtained from an autologous source. In some embodiments, the engineered immune cells are expanded in culture prior to administration to a subject in need thereof.

In some embodiments, the delivery of a cell composition (e.g., a composition including a plurality of engineered immune cells, e.g., T cells, according to any of the cells described herein) into a subject by a method or route results in at least partial localization of the cell composition at a desired site. A composition including engineered immune cells, e.g., T cells, can be administered by any appropriate route that results in effective treatment in the subject, e.g., administration results in delivery to a desired location in the subject where at least a portion of the composition delivered, e.g., at least 1×10⁴ cells, is delivered to the desired site for a period of time. Exemplary modes of suitable administration include injection, infusion, and instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is often considered a standard mode of administration.

In some embodiments, the engineered immune cells, e.g., T cells, are administered systemically, e.g., via infusion or injection. For example, a population of engineered immune cells, e.g., T cells, as described herein are administered other than directly into a target site, tissue, or organ, such that it enters, the subject's circulatory system and, thus, is subject to metabolism and other similar biological processes.

The efficacy of a treatment including any of the compositions provided herein for the prevention or treatment of a disease or health condition in a subject can be determined by a skilled clinician. However, one skilled in the art will appreciate that a prevention or treatment is considered effective if any one or all of the signs or symptoms or markers of disease are improved or ameliorated as compared to an untreated subject under similar conditions. Efficacy can also be measured by failure of a subject to worsen as assessed by decreased hospitalization or need for medical interventions (e.g., progression of the disease is halted or at least slowed). Methods of measuring these indicators are known to those of skill in the art and/or described herein. Treatment includes any treatment of a disease in a subject or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms as compared to an untreated subject under similar conditions.

Measurement of the degree of efficacy is based on parameters selected with regard to the disease being treated and the symptoms experienced. In general, a parameter is selected that is known or accepted as correlating with the degree or severity of the disease, such as a parameter accepted or used in the medical community. For example, in the treatment of a solid cancer, suitable parameters can include reduction in the number and/or size of metastases, number of months of progression-free survival, overall survival, stage or grade of the disease, the rate of disease progression, the reduction in diagnostic biomarkers (for example without limitation, a reduction in circulating tumor DNA or RNA, a reduction in circulating cell-free tumor DNA or RNA, and the like), and combinations thereof, as compared to an untreated subject under similar conditions. It will be understood that the effective dose and the degree of efficacy will generally be determined with relation to a single subject and/or a group or population of subjects. Therapeutic methods of the disclosure reduce symptoms and/or disease severity and/or disease biomarkers by at least about 1, 2, 3, 4, 5, 10, 15, 20, 25, 30, 35, 40, 45, 50, 55, 60, 65, 70, 75, 80, 85, 90, 95, 96, 97, 98, 99, or 100% as compared to an untreated subject under similar conditions.

As discussed above, a therapeutically effective amount of a pharmaceutical composition can be an amount of the pharmaceutical composition that is sufficient to promote a particular beneficial effect when administered to a subject, such as one who has, is suspected of having, or is at risk for a disease or health condition. In some embodiments, an effective amount includes an amount sufficient to prevent or delay the development of a symptom of the disease or health condition, alter the course of a symptom of the disease or health condition (for example but not limited to, slow the progression of a symptom of the disease), or reverse a symptom of the disease or health condition, as compared to an untreated subject under similar conditions. It is understood that for any given case, an appropriate effective amount can be determined by one of ordinary skill in the art using routine experimentation.

### Additional Therapies

As discussed above, any one of the compositions as disclosed herein, e.g., engineered immune cells (e.g., engineered T cells) and pharmaceutical compositions, can be administered to a subject in need thereof as a single therapy (e.g., monotherapy). In addition or alternatively, in some embodiments of the disclosure, one or more of the engineered immune cells and pharmaceutical compositions described herein can be administered to the subject in combination with one or more additional (e.g., supplementary) therapies, e.g., at least one, two, three, four, or five additional therapies. Suitable therapies to be administered in combination with the compositions of the disclosure include, but are not limited to chemotherapy, radiotherapy, immunotherapy, hormonal therapy, toxin therapy, targeted therapy, and surgery. Other suitable therapies include therapeutic agents such as chemotherapeutics, anti-cancer agents, and anti-cancer therapies.

Administration “in combination with” one or more additional therapies includes simultaneous (concurrent) and consecutive administration in any order. In some embodiments, the one or more additional therapies is selected from the group consisting of chemotherapy, radiotherapy, immunotherapy, hormonal therapy, toxin therapy, and surgery. The term chemotherapy as used herein encompasses anti-cancer agents. Various classes of anti-cancer agents can be suitably used for the methods disclosed herein. Non-limiting examples of anti-cancer agents include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

Topoisomerase inhibitors are also another class of anti-cancer agents that can be used herein. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins such as irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

Antineoplastics include the immunosuppressant dactinomycin, doxorubicin, epirubicin, bleomycin, mechlorethamine, cyclophosphamide, chlorambucil, ifosfamide. The antineoplastic compounds generally work by chemically modifying a cell's DNA.

Alkylating agents can alkylate many nucleophilic functional groups under conditions present in cells. Cisplatin and carboplatin, and oxaliplatin are alkylating agents. They impair cell function by forming covalent bonds with the amino, carboxyl, sulfhydryl, and phosphate groups in biologically important molecules.

Vinca alkaloids bind to specific sites on tubulin, inhibiting the assembly of tubulin into microtubules (M phase of the cell cycle). The vinca alkaloids include: vincristine, vinblastine, vinorelbine, and vindesine.

Anti-metabolites resemble purines (azathioprine, mercaptopurine) or pyrimidine and prevent these substances from becoming incorporated in to DNA during the “S” phase of the cell cycle, stopping normal development and division. Anti-metabolites also affect RNA synthesis.

Plant alkaloids and terpenoids are obtained from plants and block cell division by preventing microtubule function. Since microtubules are vital for cell division, without them, cell division cannot occur. The main examples are vinca alkaloids and taxanes.

Podophyllotoxin is a plant-derived compound which has been reported to help with digestion as well as used to produce two other cytostatic drugs, etoposide and teniposide. They prevent the cell from entering the G1 phase (the start of DNA replication) and the replication of DNA (the S phase).

Taxanes as a group includes paclitaxel and docetaxel. Paclitaxel is a natural product, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

In some embodiments, the anti-cancer agents can be selected from remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, gefitinib (Iressa®), taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bortezomib (Velcade®), bisphosphonate, arsenic trioxide, vincristine, doxorubicin (Doxil®), paclitaxel, ganciclovir, adriamycin, estrainustine sodium phosphate (Emcyt®), sulindac, etoposide, and combinations of any thereof.

In other embodiments, the anti-cancer agent can be selected from bortezomib, cyclophosphamide, dexamethasone, doxorubicin, interferon-alpha, lenalidomide, melphalan, pegylated interferon-alpha, prednisone, thalidomide, or vincristine.

In some embodiments, the methods of prevention and/or treatment as described herein further include an immunotherapy. In some embodiments, the immunotherapy includes administration of one or more checkpoint inhibitors. Accordingly, some embodiments of the methods of treatment described herein include further administration of a compound that inhibits one or more immune checkpoint molecules. Non-limiting examples of immune checkpoint molecules include CTLA4, PD-1, PD-L1, A2AR, B7-H3, B7-H4, TIM3, and combinations of any thereof. In some embodiments, the compound that inhibits the one or more immune checkpoint molecules includes an antagonistic antibody. Examples of antagonistic antibodies suitable for the compositions and methods disclosed herein include, but are not limited to, ipilimumab, nivolumab, pembrolizumab, durvalumab, atezolizumab, tremelimumab, and avelumab.

In some aspects, the one or more anti-cancer therapy is radiation therapy. In some embodiments, the radiation therapy can include the administration of radiation to kill cancerous cells. Radiation interacts with molecules in the cell such as DNA to induce cell death. Radiation can also damage the cellular and nuclear membranes and other organelles. Depending on the radiation type, the mechanism of DNA damage may vary as does the relative biologic effectiveness. For example, heavy particles (i.e. protons, neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray. Radiation also contemplated herein includes, for example, the directed delivery of radioisotopes to cancer cells. Other forms of DNA damaging factors are also contemplated herein such as microwaves and UV irradiation.

Radiation may be given in a single dose or in a series of small doses in a dose-fractionated schedule. The amount of radiation contemplated herein ranges from about 1 to about 100 Gy, including, for example, about 5 to about 80, about 10 to about 50 Gy, or about 10 Gy. The total dose may be applied in a fractioned regime. For example, the regime may include fractionated individual doses of 2 Gy. Dosage ranges for radioisotopes vary widely, and depends on the half-life of the isotope and the strength and type of radiation emitted. When the radiation includes use of radioactive isotopes, the isotope may be conjugated to a targeting agent, such as a therapeutic antibody, which carries the radionucleotide to the target tissue (e.g., tumor tissue).

Surgery described herein includes resection in which all or part of a cancerous tissue is physically removed, exercised, and/or destroyed. Tumor resection refers to physical removal of at least part of a tumor. In addition to tumor resection, treatment by surgery includes laser surgery, cryosurgery, electrosurgery, and microscopically controlled surgery (Mohs surgery). Removal of pre-cancers or normal tissues is also contemplated herein.

Accordingly, in some embodiments, a composition according to the present disclosure is administered to the subject individually as a single therapy (monotherapy) or as a first therapy in combination with at least one additional therapies (e.g., second therapy). In some embodiments, the second therapy is selected from the group consisting of chemotherapy, radiotherapy, immunotherapy, hormonal therapy, toxin therapy, targeted therapy, and surgery. In some embodiments, the second therapy is selected from the group consisting of chemotherapy, radiotherapy, immunotherapy, hormonal therapy, toxin therapy or surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

Each of the aspects and embodiments described herein are capable of being used together, unless excluded either explicitly or clearly from the context of the embodiment or aspect.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### Examples

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

Additional embodiments are disclosed in further detail in the following examples, which are provided by way of illustration and are not in any way intended to limit the scope of this disclosure or the claims.

### Example 1. General Materials and Methods

Viral Vector Construction: MSGV retroviral vectors encoding the following CARs were previously described: CD19-28z, CD19-BBz, GD2-BBz and Her2-BBz (Neelapu et al. 2017). HA-28z CAR was created as previously described in (Rachel et al) by introduction of a point mutation into the 14G2a scFv of the GD2-28z CAR plasmid to create the E101K mutation.

T Cell Isolation: Healthy donor buffy coats were purchased from the Stanford Blood Center under an IRB-exempt protocol. Primary human T cells were isolated using the RosetteSep Human T cell Enrichment kit (Stem Cell Technologies) according to the manufacturer's protocol. Isolated T cells were cryopreserved in CryoStor CS10 cryopreservation medium (Stem Cell Technologies). CD39− T cells were purified using anti-PE MicroBeads (Miltenyi Biotec) and LD autoMACS (Miltenyi Biotec) columns according to the manufacturer's protocol. Depletion efficiency was assessed by flow cytometry.

Human CAR T Cell Production: Non-tissue culture treated 12-well plates were coated overnight at 4° C. with 1 ml Retronectin (Takara) at 25 g/ml in PBS. Plates were washed with PBS and blocked with 2% BSA for 15 minutes. Thawed retroviral supernatant was added at approximately 1 ml per well and centrifuged for 2 hours at 32° C. at 3,200 rpm before the addition of cells. Primary human T cells were thawed and activated with Human T-Expander CD3/CD28 Dynabeads (Gibco) at a 3:1 bead:cell ratio in complete medium (RPMI 1640 supplemented with 10% fetal bovine serum, 10 mM N-2-hydroxyethylpiperazine-N9-2-ethanesulfonic acid, 2 mM GlutaMAX, 100 U/mL penicillin (Gibco), and 100 U/mL (Peprotech). T cells were transduced with retroviral vector on Days 2 and 3 post-activation. Beads were taken off at Day 4 post-activation.

Cell Lines: The CD19+ Nalm6-GL B-ALL cell line was provided by D. Barrett (Barrett 2011). Nalm6-GD2 was created by co-transducing Nalm6-GL with cDNAs for GD2 synthase and GD3 synthase. All cell lines were cultured in complete media (CM) (RPMI supplemented with 10% FBS, 10 mM HEPES, 2 mM GlutaMAX, 100 U ml-1 penicillin, and 100 g ml-1 streptomycin (Gibco)). STR DNA profiling of all cell lines was conducted by Genetica Cell Line testing once per year. None of the cell lines used in this study was included in the commonly misidentified cell lines registry. Before using for in vivo experiments, cell lines were tested with MycoAlert detection kit (Lonza). All cell lines tested negative.

Flow Cytometry: The anti-CD19 CAR idiotype antibody was provided by B. Jena and L. Cooper. The 1A7 anti-14G2a idiotype antibody was obtained from NCI Frederick and University of Texas M.D. Anderson Cancer Center. Her2 CARs was detected using human Her2-Fc recombinant proteins (R&D). The idiotype antibodies and Fc-fusion protein were conjugated in house with Dylight650 antibody labelling kits (Thermo Fisher). T cell surface phenotype was assessed using the following antibodies:
From BioLegend: CD4-APC-Cy7 (clone OKT4), CD8-PerCp-Cy5.5 (clone SK1), TIM-3-BV510 (clone F38-2E2), CD39-FITC, PE or APC-Cy7 (clone A1), CD3-PacBlue (clone HIT3a);
From eBioscience: PD-1-PE-Cy7 (clone eBio J105), LAG-3-PE (clone 3DS223H), CD45RO-PE-Cy7 (clone UCHL1), CD45-PerCp-Cy5.5 (clone HI30), CCR7-PE (clone 3D12); and
From BD: LAG-3-BV421 (clone T47-530), CD45RA-FITC or BV711 (clone HI100), CD62L-BV605 (clone DREG-56), CD73-PE-Cy7 or BV510 (clone AD2), CD4-BUV395 (clone SK3), CD8-BUV805 (clone SK1).

Proliferation Assay: T cells were labeled with 2.5 μM CTV at 37° C. for 10 min followed by addition of 5 ml ice cold PBS 2% FBS to quench the reaction. Next, cells were washed with complete RPMI 1640 and 5×10⁴ cells were activated and cultured in the 96-well plates coated overnight at 4° C. with 1 or 5 μg/ml CD19 or 1A7 idiotype. Proliferation assay was performed in the absence of exogenous IL-2. After 72-96 hours, CTV dilution, as an indicator of cell proliferation, was assessed by flow cytometry.

Suppression Assay: For assessing IL-2 secretion inhibition 5×10⁴ CD19 CAR T cells 5×10⁴ Nalm6 tumor cells together with 5×10⁴ of HA CAR or Mock T cells were cultured in 300 μL CM in 96-well flat bottom plates for 24 h. Triplicate wells were plated for each condition. Culture supernatants were collected and analyzed for IL-2 by ELISA (BioLegend).

Co-culture Assays: CAR-T cells were cultured with 1 μM of CPI444 (CORVUS BIOPHARMA) for 2 to 24 hours before the duration of co-culture (unless stated otherwise) with tumor cells or plate-bound idiotype at the concentration 1 or 5 μg/ml. To stimulate a2aR, cells were treated with 0.01-0.1 mM of NECA (Torcis). For cytotoxicity assays, approximately 5×10⁴ of tumor cells were co-cultured with CAR T cells at indicated ratio in 200 μL CM in 96-well flat bottom plates. Four images per well at 10× zoom were collected at each time point. Tumor cell growth was quantified by measuring total integrated GFP intensity per well using an IncuCyte ZOOM Live-Cell analysis system (Essen Bioscience) every 2-3 hr. GFP signal was normalized to the time 0 signal. Cell culture supernatants were collected at 24 hours, and interleukin-2 (IL-2) and interferon-g concentrations were determined by enzyme-linked immunosorbent assay (Biolegend). Triplicate wells were plated for each condition. All the co-culture experiments were performed between Day 10 and 16 post-activation unless stated differently in the text.

CRISPR Knockout: CRISPR-Cas9 gene knockout was performed by transient Cas9/gRNA (RNP) complex electroporation using the P3 Primary Cell 4D-Nucleofector X Kit S (Lonza). On Day 4 of culture, HA-28z CAR T cells were counted, pelleted and resuspended in P3 buffer at 1.5×10⁶-2×10⁶ cells per 18 μL reaction. 3.3 μg Alt-R.Sp (Streptococcus pyogenes) Cas9 nucleases. Cas9 protein (IDT) and 120 pmol chemically modified synthetic sgRNA (Synthego) (6:1 molar ratio gRNA:Cas9) per reaction was pre-complexed for 10 min at room temperature to create ribonucleoprotein complexes (RNP). A 18-μL cell suspension was mixed with RNP and electroporated using the EO-115 protocol in 16-well cuvette strips. Cells were recovered at 37° C. for 30 min in 200 L T cell medium then expanded as described above. Knockdown efficiency was determined using TIDE and/or flow cytometry. Control HA-28z CAR T cells were electroporated with a gRNA targeting the safe harbor locus AAVS1. The following gRNA target sequences were used:

AAVS1:
(SEQ ID NO: 1)
GGGGCCACTAGGGACAGGAT;
ADORA2a-guide1:
(SEQ ID NO: 2)
GUCUGUGGCCAUGCCCAUCA;
ADORA2a-guide2:
(SEQ ID NO: 3)
UACACCGAGGAGCCCAUGAU;
CD73-guide1:
(SEQ ID NO: 4)
GCGGGCGCCCGCGCGGCUCG;
CD73-guide2:
(SEQ ID NO: 5)
CUAUGUGUCCCCGAGCCGCG;
and
CD39:
(SEQ ID NO: 6)
UGGCACCCUGGAAGUCAAAG.

Bulk RNA-Seq: For bulk RNA isolation, healthy donor T cells were prepared as described above. On Day 14 CD39+ and CD39− CD4+ or CD8+ subsets were isolated using a BD FACSAria cell sorter (Stem Cell FACS Core, Stanford University School of Medicine) and total mRNA was isolated using Qiagen RNEasy Plus mini isolation kit. Bulk RNA-seq was performed by BGI America (Cambridge, MA) using the BGISEQ-500 platform, single-end 50-bp read length, at 30×10⁶ reads per sample. Significantly different genes were identified by DESeq2 using Wald test. Gene annotation enrichment analysis was performed using Kegg pathways and GO terms (biological process, cellular component, and molecular function). Functional annotation clustering was performed, and terms with p<0.05 (Benjamini corrected) were shown. Redundant terms were manually removed for visualization.

ATP Measurements: CAR T cells were washed with RPMI without phenol red (Agilent). Next, 5×10⁴ of cells were resuspended in 150 μL of phenol free RPMI media and spiked with 20 μM of ATP (PerkinElmer) in the presence. After 10 min of incubation at 37° C. supernatants were collected and concentration of ATP/sample was measured using ATPlite Luminescence Assay System (PerkinElmer) according to the manufacturer's protocol.

ADO Measurements: To measure the ability of ectoenzymes at the surface CAR T cells were treated with CPX006 24 hrs and 2 hrs before the assay. Next, cells were washed with RPMI without phenol-free (Agilent). Next, 5×10 of cells were resuspended in 150 μl of phenol-free RPMI media and spiked with 20 μM of ATP (PerkinElmer). After 30 min of incubation at 37° C., supernatants were collected. For autocrine production of adenosine, 3×10⁵ CAR T cells were resuspended in 120 μL of phenol-free RPMI (Agilent) and incubated at 37° C. for 2 hrs. Adenosine concentration was assessed using Adenosine Assay Kit (Abcam) according to the manufacturer's protocol.

Luminex: At Day 14 post-activation, sorted CD39+ and CD39− CD4+ or CD8+ CAR T cells were co-cultured with Nalm6-GD2 cells at 1:1 ratio. Duplicate wells were plated for each condition. After 24 hrs, supernatants were collected and analyzed using Luminex assay was performed at the Human Immune Monitoring Center of Stanford University. Human 62-plex kits were purchased from eBioscience/Affymetrix and used according to the manufacturer's recommendations with modifications as described. Briefly, beads were added to a 96-well plate and washed in a BioTek ELx405 Select Deep Well Washer. Samples were added to the plate containing the mixed antibody-linked beads and incubated at room temperature for 1 h followed by overnight incubation at 4° C. with shaking. The cold and room temperature incubation steps were performed on an orbital shaker at 500-600 rpm. After overnight incubation, plates were washed in a BioTek ELx405 Select Deep Well Washer; then, biotinylated detection antibody was added for 75 min at room temperature with shaking. The plate was washed as described earlier and streptavidin-PE was added. After incubation for 30 min at room temperature, washing was performed as described earlier and reading buffer was added to the wells. Plates were read using a Luminex FLEXMAP 3D instrument with a lower bound of 50 beads per sample per cytokine. Custom assay control beads by Radix Biosolutions were added to all wells. The dilution factor was accounted for. For each cytokine, concentration (pg/ml) was calculated. Heatmaps were generated using the GraphPad Prism 8.4.

Seahorse Mito Stress Assay: A Seahorse XFe96 Bioanalyser (Agilent) was used to determine OCR and ECAR for HA and ADA O/E HA CAR T cells. Cells were washed with assay media (XF Base media (Agilent) with glucose (25 mM), sodium pyruvate (1 mM) and L-glutamine (2 mM) (Gibco), pH 7.4 at 37° C.) before being plated onto Seahorse cell culture plates coated with Cell-Tak (Corning) at 2×10⁵ cells per well. After adherence and equilibration, cell OCR and ECAR was measured during a Seahorse Mito Stress assay (Agilent), with addition of oligomycin (1.5 μM), carbonyl cyanide 4-(trifluoromethoxy)phenylhydrazone (FCCP; 1.0 μM) and antimycin A and rotenone (0.5 M each)).

Statistical Analysis: Unless otherwise noted, statistical analyses for significant differences between groups were conducted using unpaired two-tailed t-tests without correction for multiple comparisons and without assuming consistent s.d. using GraphPad Prism 8.4.

### Example 2. CD39 Expression Correlates with Progressive Loss of Function During CAR T Cell Exhaustion

This Example describes the results of experiments performed to demonstrate that CD39 expression correlated with progressive loss of function during CAR T cell exhaustion.

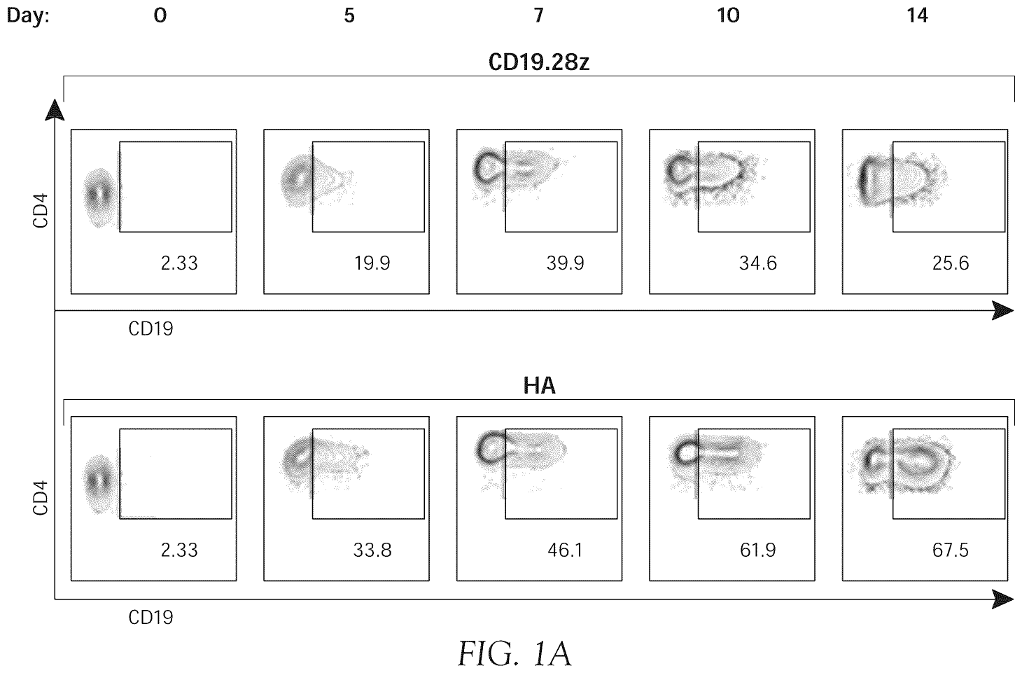

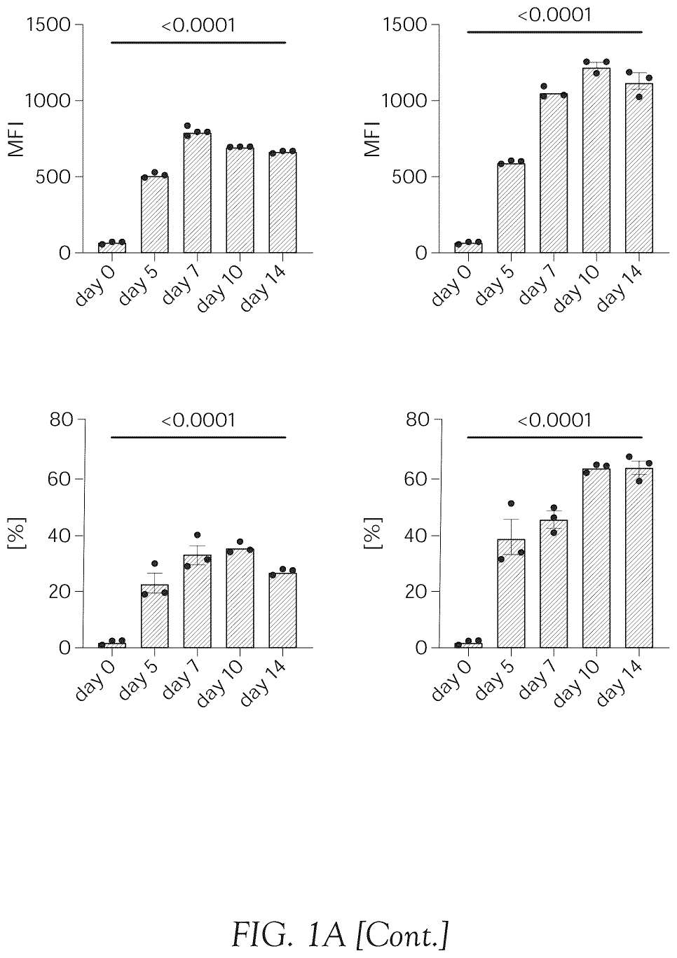

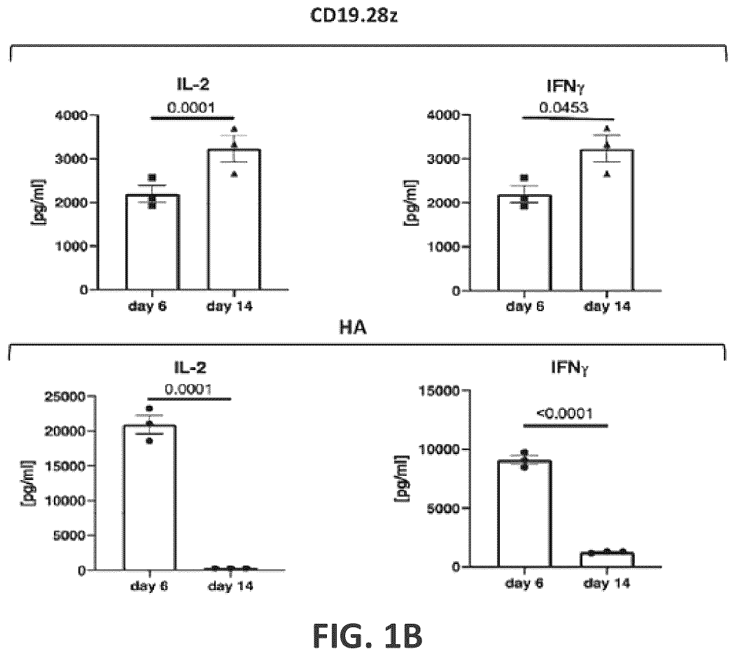

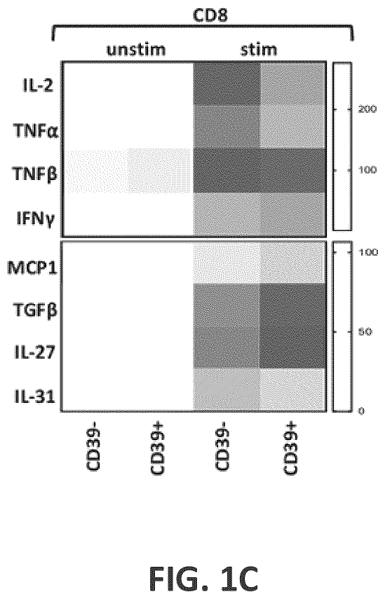

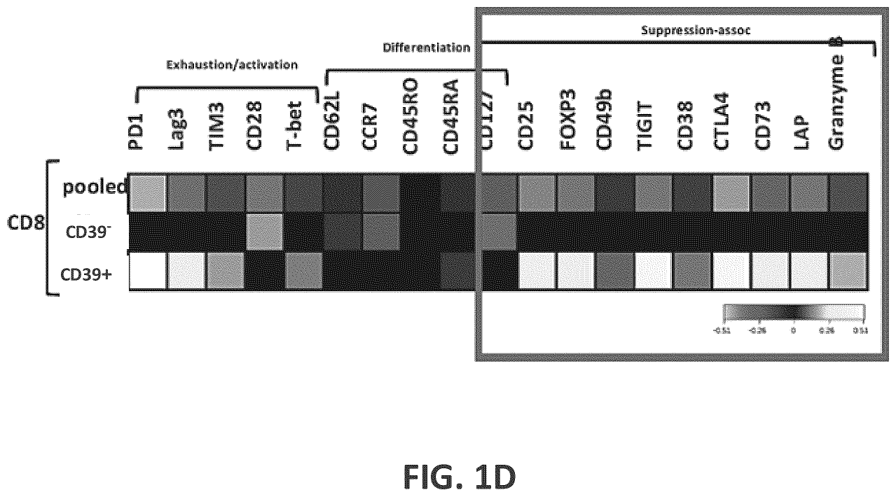

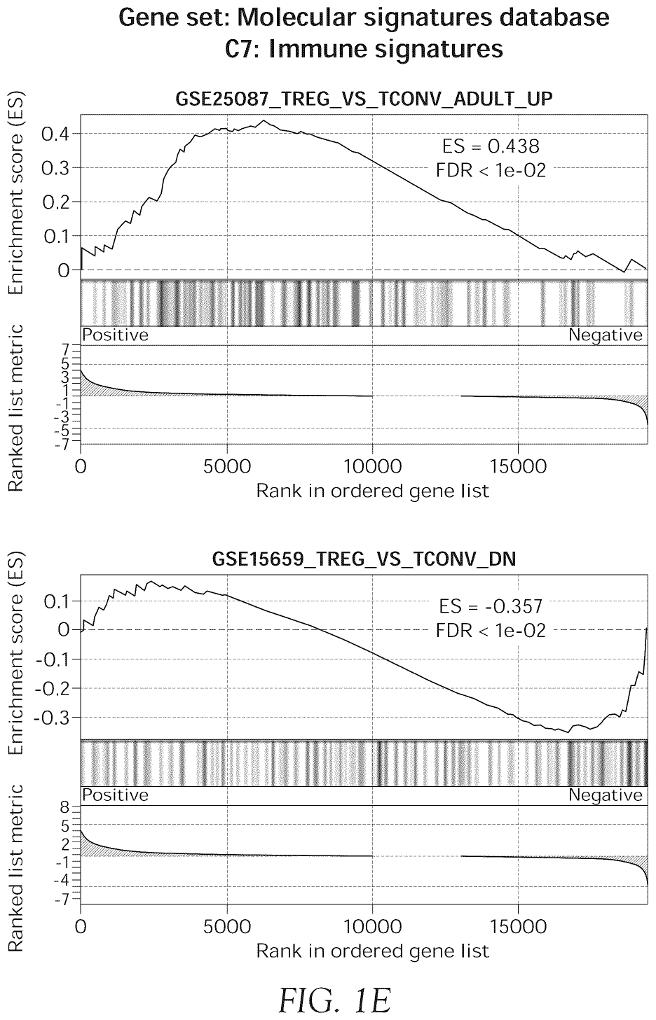

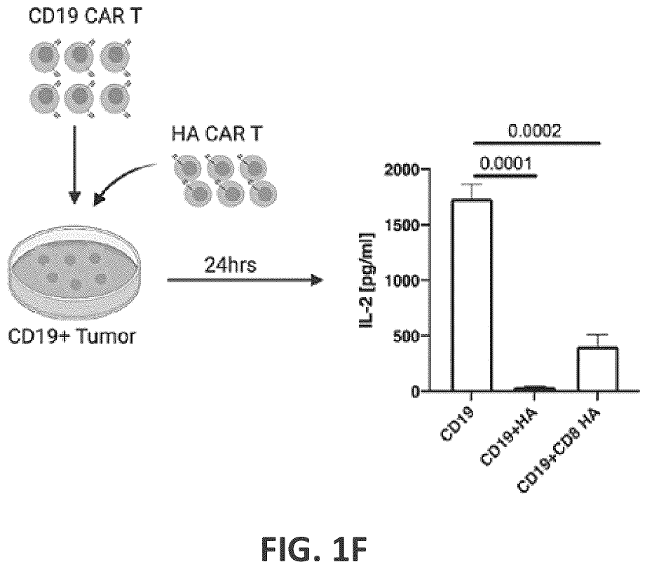

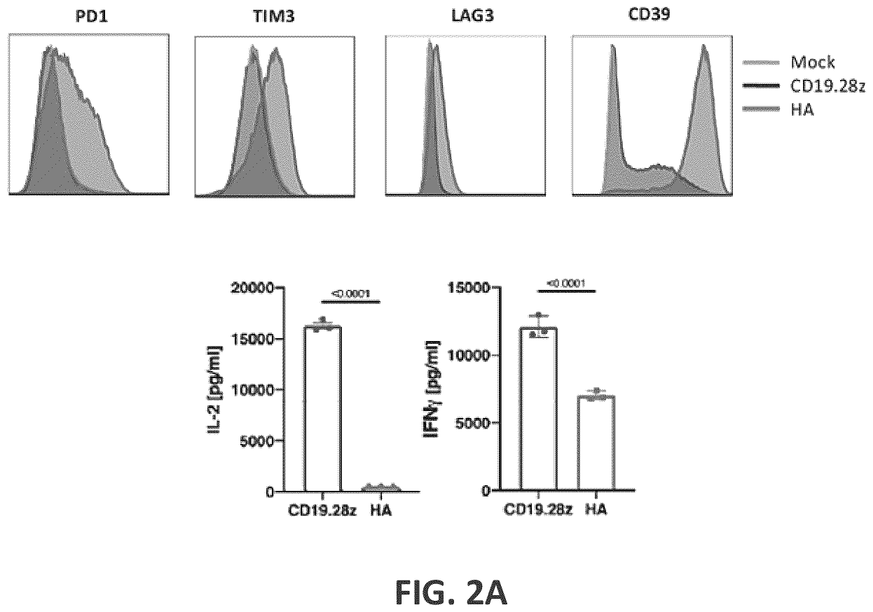

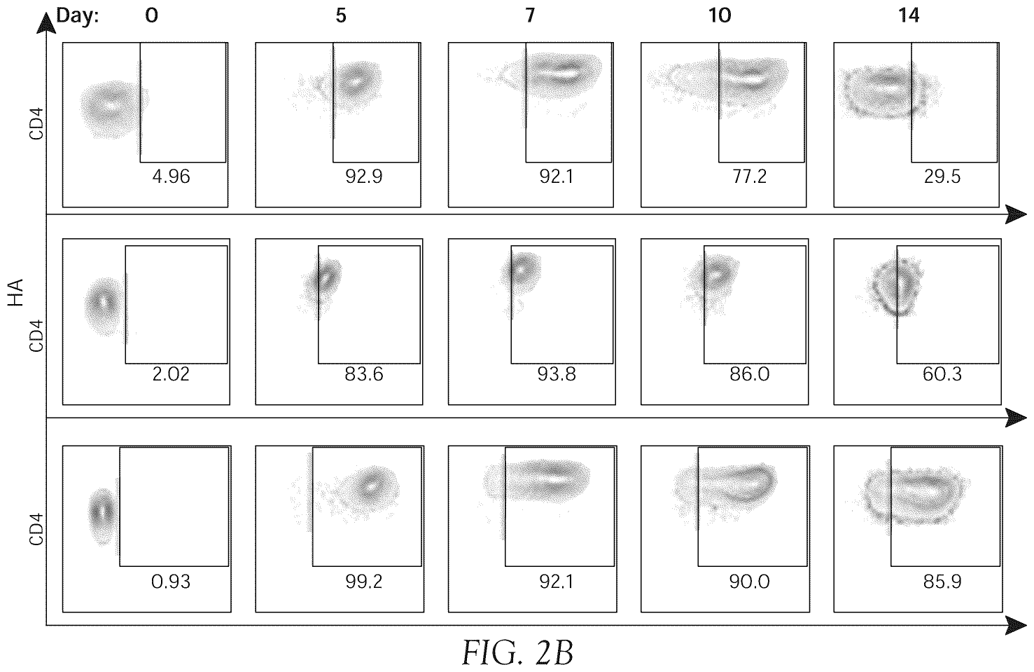

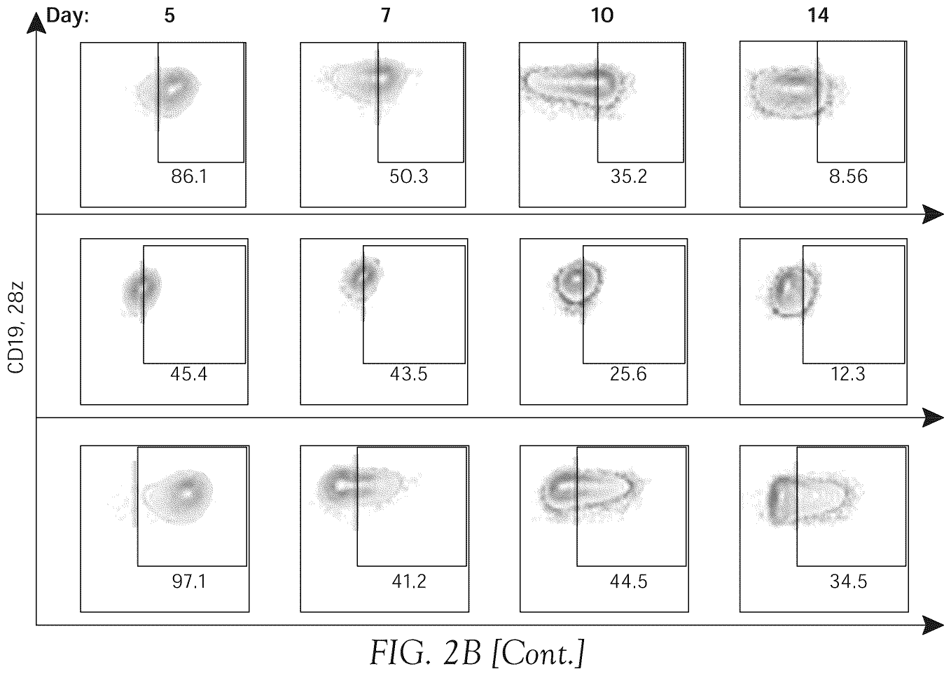

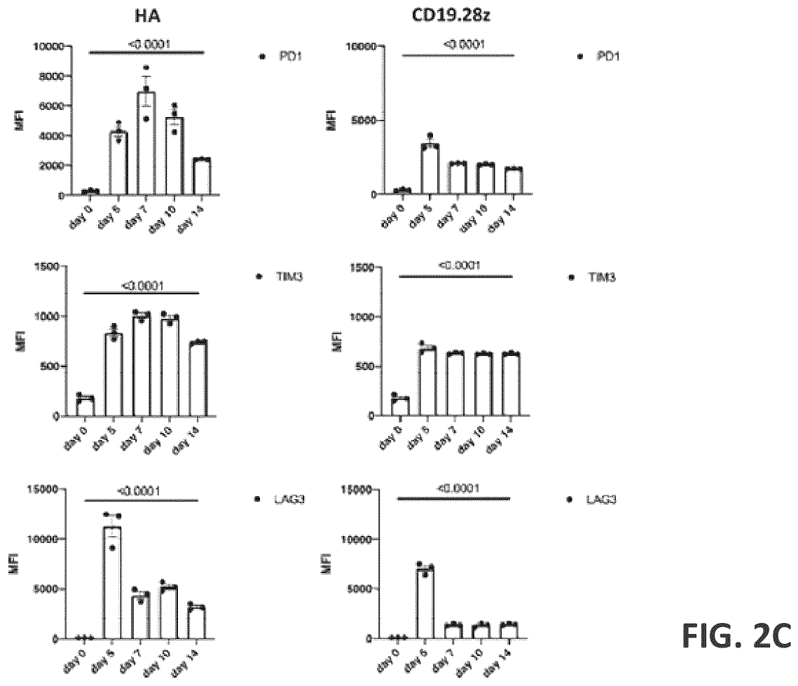

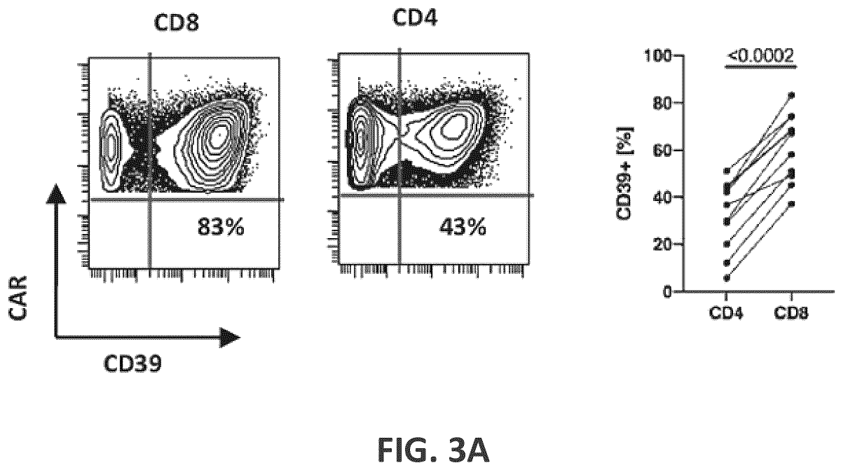

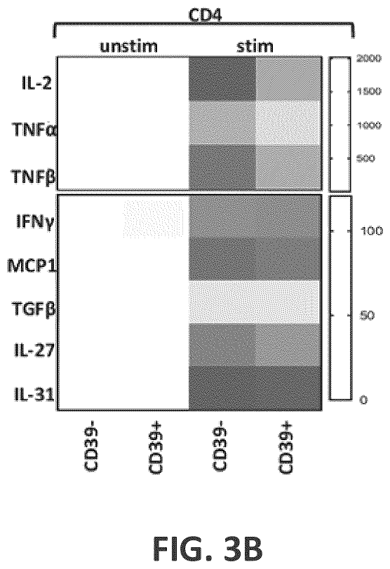

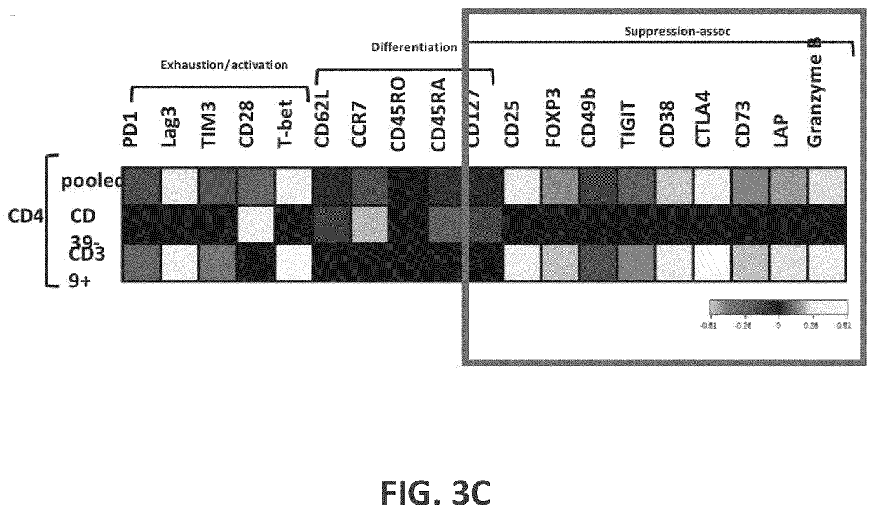

Human T cells expressing a high-affinity (HA) CAR targeting disialoganglioside GD2 (HA-CAR) that tonic signals in the absence of antigen exhibited decreased effector cytokine secretion and high surface expression of inhibitory receptors (FIG. 2A). Using this model, it was observed that CD39 expression on HA-CAR T cells strongly correlated with acquisition of others features of exhaustion. However, the kinetics of CD39 were different to that of other exhaustion markers. Canonical markers of T cell exhaustion such as TIM3, PD1, or LAG3, showed an expression and frequency peak at Days 5-7 post-activation, subsequently followed by a slow downregulation as activation-bead-induced signaling wanes (FIG. 2B). Conversely, CD39 expression and frequency peaked once the exhaustion phenotype was fully established (FIG. 1A). Although, the expression of exhaustion markers was still higher at the later time points as compared to non-tonically signaling CAR T cells (FIG. 1A; FIG. 2B). This delayed increase in the frequency of CD39 expression correlated as well with a significant/progressive decrease in secretion of effector cytokines IL-2 (p=0.0001) and IFNγ (p<0.0001) (FIG. 1B). Notably, CD39 expression appeared to be more specific to CD8 CAR T cells (61.3%±4.3) as compared to CD4 CAR T cells (32.5%±4.4) (FIG. 3A).

To assess if CD39 expression was indicative of diminished CAR T functionality, CD39+HA CAR T cells were co-cultured with Nalm6 leukemic cells engineered to overexpressed GD2 on their surface and subsequently measured the levels of cytokines secretion. As expected, data showed lower secretion of proinflammatory cytokines, such as IL-2, TNFα, and TNFβ, or IL-31 (belonging to IL-6 family) by CD39+ CAR T cells as compared to their CD39− counterparts (FIG. 1C). In contrast, IFNγ involved in immune cell migration and MPC-1 showed increased secretion (FIG. 1C). Notably, TGFβ and IL-27, both cytokines involved in differentiation and suppressive function of regulatory T cells, were secreted at higher levels by CD39+CD8 CAR T cells. A similar trend was observed in CD4+ CAR T cells (FIG. 3B).

Taken together, these results demonstrate that CD39+ was a distinctive dysfunctional/exhausted T cell population.

### Example 3. CD39+CD8+ Exhausted CAR T Cells Exhibit Tree-Associated Phenotype and Suppression Function

This Example describes the results of experiments performed to demonstrate that CD39+CD8+ exhausted CAR T cells exhibited Treg-associated phenotype and suppression function.

To better characterize CD39+ CAR-T cell population, a high-dimensional single cell mass cytometry was used (FIG. 1D). Higher expression of canonical exhaustion markers, such as TIM3, PD1, LAG3, was observed. Exhaustion associated transcriptional factor T-bet in CD39+CD8 CAR T cells was also observed. Notably, CD39+ CAR-T cells exhibited low or no expression of memory and homing molecules like CD62L, CCR7, or CD127, indicative of a more differentiated phenotype. However, in accordance with cytokine secretion pattern shown in FIG. 1C, markers associated with immunosuppression such as Foxp3, TIGIT, CD49, LAP, CD73, or CTLA4 were upregulated (marked with a box). Similar results in CD4+HA CAR T cells were obtained (FIG. 3C).

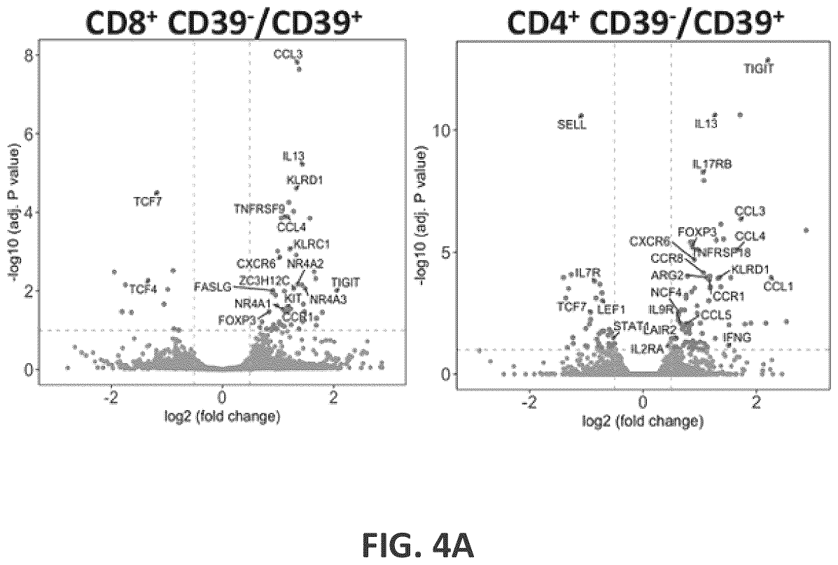

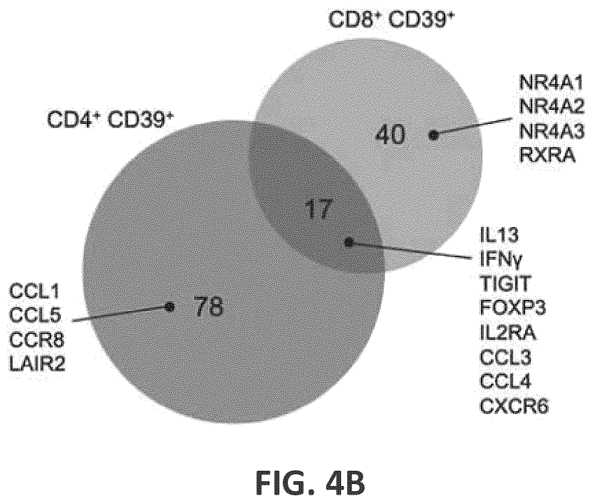

Genome-wide transcriptomic analysis confirmed that both CD4+CD39+ and CD8+CD39+ CAR-T cells expressed lower levels of memory/homing-associated genes such as (TCF7, TCF4, Sell or IL-7R) as compared to their CD39− counterparts. It also confirmed that CD39+ CAR-T cells expressed higher levels many Treg-associated genes (FIGS. 4A-4B). Gene Set Enrichment Analysis (GSEA) showed significant similarities in gene expression pattern between CD39+CD8 CAR T cells and regulatory T cells (FIG. 1E). It was observed that CD39+ T cells expressed high level of nuclear orphan receptors involved in inducing FOXP3 in regulatory T cells and retinoic X receptor alpha (RXRA), which has been reported to be involved in differentiation of Foxp3+ induced regulatory T cells.

To examine if exhausted CAR T cells exhibited not only phenotypical but also functional similarities to Tregs, their capacity to suppress the function of surrounding cells was assessed. CD19 CAR T cells with 4-1BBz co-stimulatory domain that demonstrated increased clinical efficacy and persistence were chosen to test their capacity to suppress the function of surrounding cells. IL-2 secreted by CD19.BBz CAR T cells co-cultured with Nalm6 leukemia cells was measured in the presence or absence of bulk or sorted CD8+HA CAR T cells for 24 hours. CD19.BBz CAR T cells activated in the presence of either bulk or CD8+HA T cells secreted significantly less IL-2, indicating that HA CAR-T cells can suppress antigen-dependent IL-2 production of neighboring healthy CAR-T cells (FIG. 1F).

Together, these data demonstrated that CD39+CD8 CAR T cells were not only exhausted, but they can represent a novel cell subpopulation with enriched suppressive molecular signature, phenotype and function.

### Example 4. Conversion of CD39− CAR T Cell Population into CD39+ Depends on Tonic Signaling

This Example describes the results of experiments performed to demonstrate that conversion of CD39− CAR T cell population into CD39+ depended on tonic signaling.

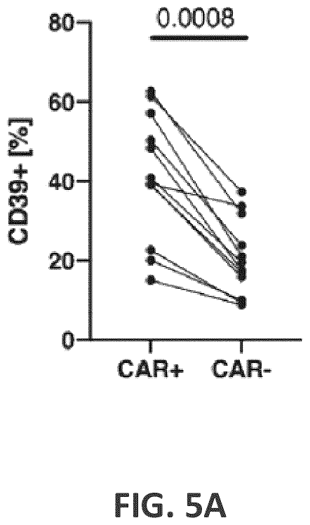

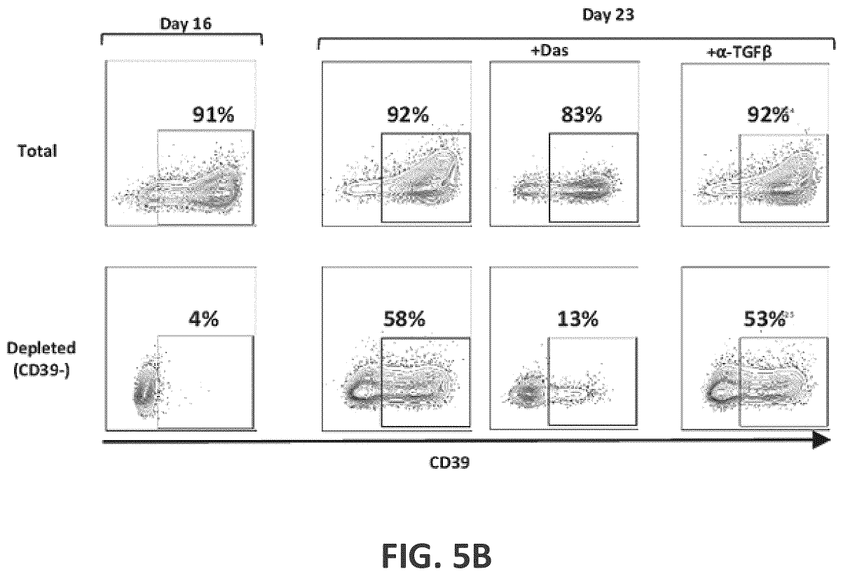

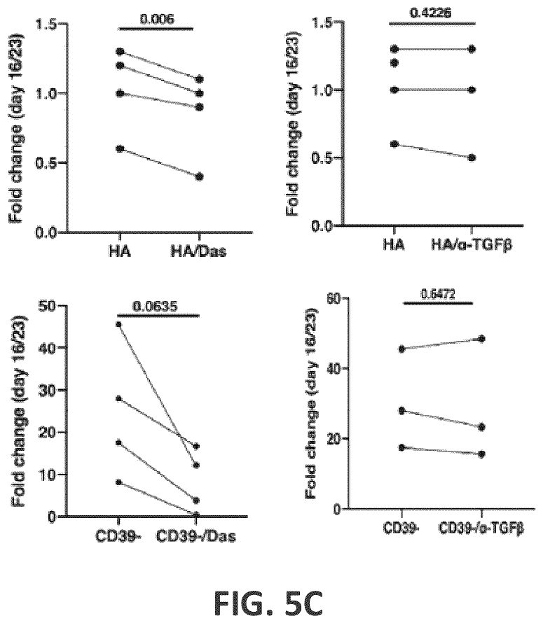

CD39 has been previously described as a marker of a regulatory T cell subset that can prevent effective anti-tumor immune responses in tumor-bearing hosts. In the antigen-independent CAR-driven exhaustion model presented herein, the frequency of CD39+ T cells was increased in cells with the HA CAR (FIG. 5A). This increase in CD39+ population can be due to the expansion of small pre-existing CD39+ population in the culture, or due to CAR− mediated factors triggered by tonic signaling. To distinguish between the two possibilities, CD39− HA CAR T cells were sorted and maintained in the presence or absence of the tyrosine kinase inhibitor dasatinib, which was recently shown to block tonic signaling without affecting CAR T cell proliferation (FIGS. 5B-5C). Addition of dasatinib to CD39-depleted HA CAR-T cultures severely affected their ability to generate CD39+HA CAR T cells. This result indicates that CAR-mediated signaling and exhaustion was required for upregulation of a CD39 and generation of a CD39+ T cell subset.

It has been shown that TGFβ plays a prominent role in the upregulation of CD39 in the context of regulatory T cells. Whether the TGFβ produced by exhausted cells was sufficient to drive conversion of CD39− cells into CD39+ cells was investigated. It was observed that addition of neutralizing anti-TGFβ antibody to bulk and CD39-depleted cultures did not affect the frequency of CD39+ cells, nor the expression levels of CD39 on CAR T cells (FIGS. 5B-5C).

These data suggested that chronic T cell stimulation was sufficient to convert CD39-cells into CD39+ cells.

### Example 5. Exhausted HA CAR T Cells Exhibit High Expression of Enzymatically Active CD39 and CD73 which Result in Production of Suppressive Adenosine

This Example describes the results of experiments performed to demonstrate that exhausted HA CAR T cells exhibited high expression of enzymatically active CD39 and CD73 which resulted in production of suppressive adenosine.

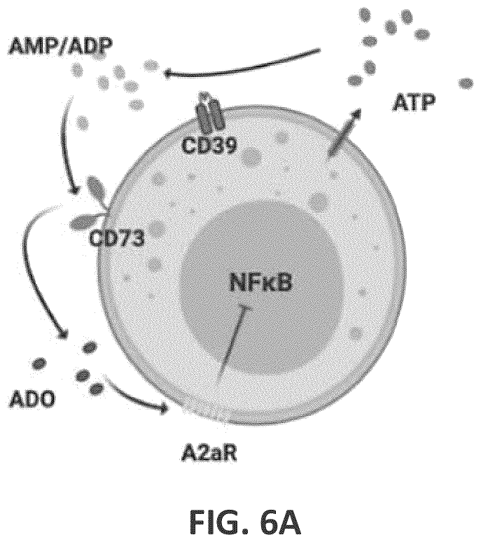

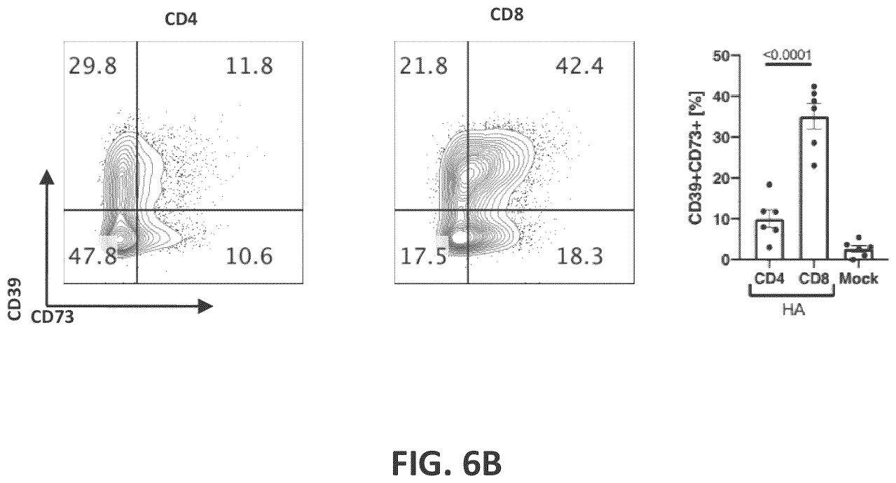

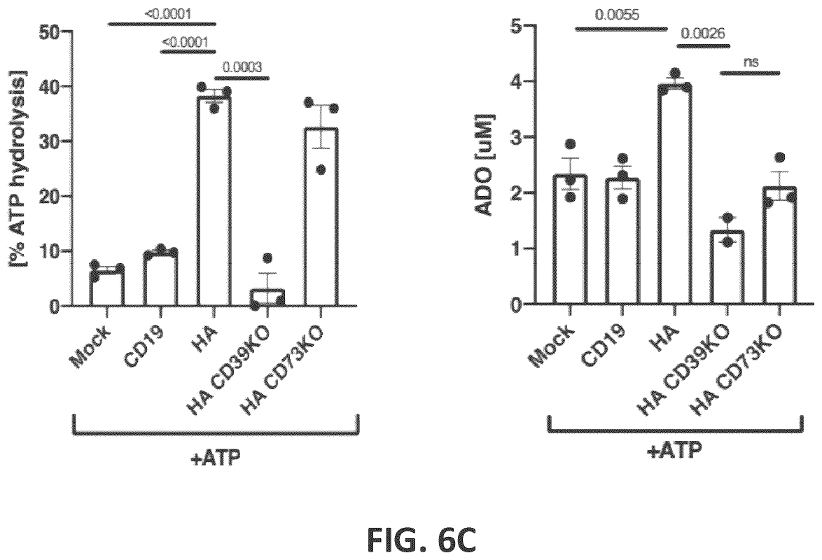

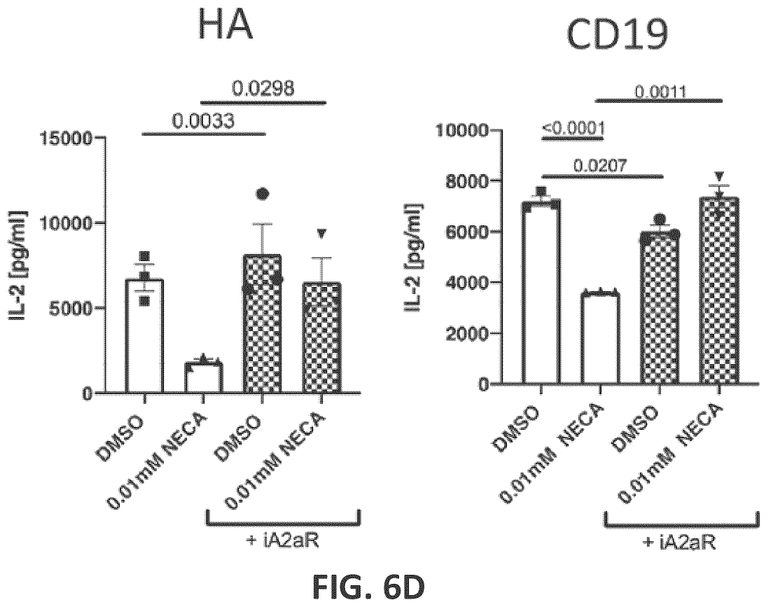

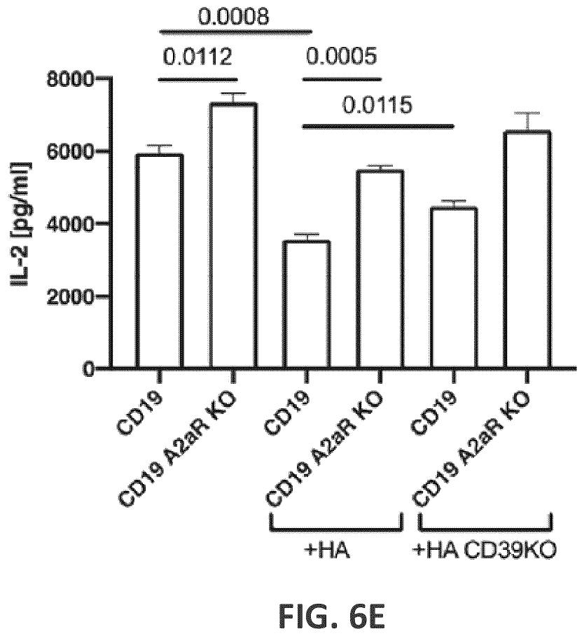

One of the mechanisms of Treg immunosuppression is co-expression of CD39 and CD73 that results in increased levels of extracellular adenosine, consequently suppressing effector T cells through the activation of high affinity A2a adenosine receptors (FIG. 6A). Vast majority of murine CD4+ Tregs express CD39 and CD73 at high levels, however only small proportion of human Treg cells are CD73+. Surprisingly, in the presently described tonic signaling CAR model, not only was the frequency of CD39+/CD73+ population much lower in CD4+ subset than CD8+, but also the levels of expression of CD73 in this subset was much lower in CD4+ subset than CD8+(FIG. 6B). Subsequently, whether these enzymes were functional was investigated by assessing the ability of these cells to hydrolyze extracellular ATP and to generate adenosine. Consistent with higher expression of CD39, HA CAR T cells showed a 4-fold increased capacity to hydrolyze eATP as compared to CD19 CAR or mock T cells (40% vs 10%±1.245 SEM). Using the CRISPR/Cas9 system to knock out CD39 or CD73, CD39 and not CD73 was crucial for conversion of eATP into ADP/AMP was confirmed (FIG. 6C). Next, whether exhausted CAR T cells could further process ADP/AMP into adenosine was investigated. It was observed that HA CAR T cells were able produce twice as much adenosine as compare to mock or non-exhausted CD19 CAR T cells (4 μM vs 2 μM±0.2965SEM) (FIG. 6C). Importantly, adenosine production was abrogated by knockout of either CD39 or CD73.

Taken together, these data indicates that higher levels of expression of CD39 and CD73 on the surface of exhausted HA CAR-T cells correlated with increased capacity to degrade ATP and transform ADP/AMP into adenosine.

### Example 6. Adenosine can Suppress Cytokine Production by CAR T Cells after Antigen Stimulation

This Example describes the results of experiments performed to demonstrate that adenosine can suppress cytokine production by CART T cells after antigen stimulation.

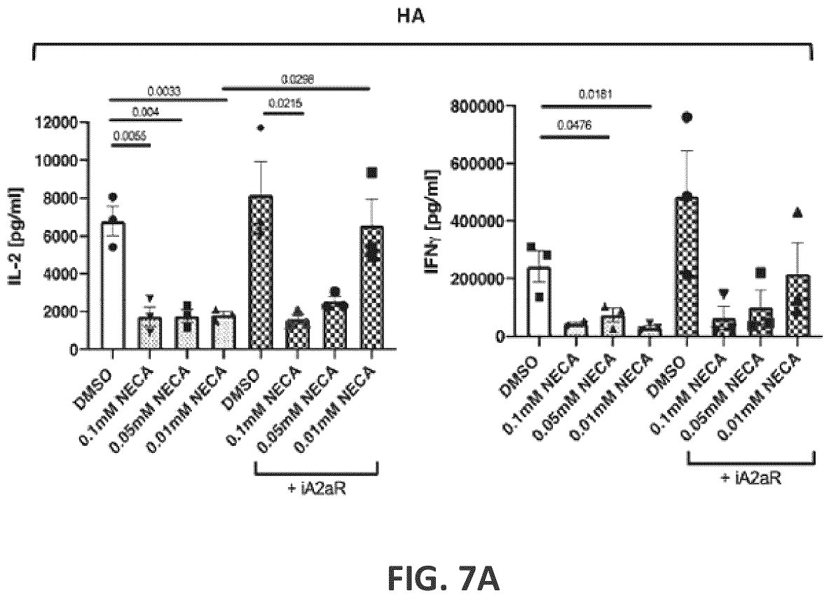

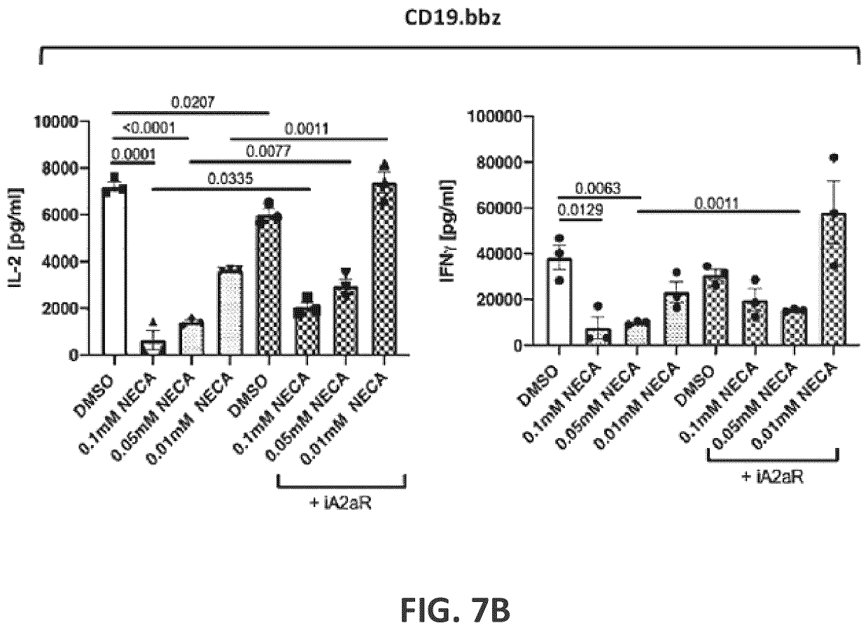

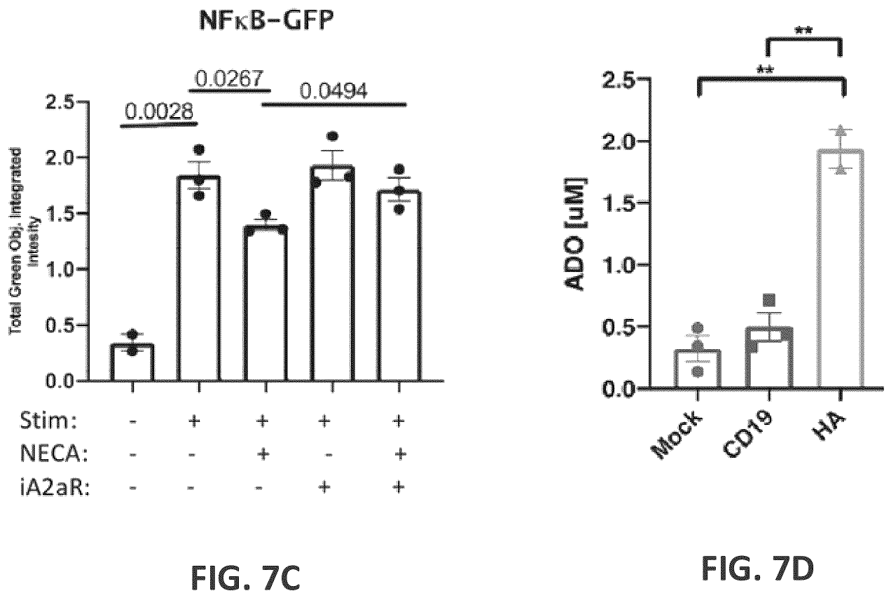

To test whether CAR T cells are susceptible to adenosine-mediated suppression, HA and CD19 CAR T cells were activated with plate-bounded idiotype in the presence or absence of the adenosine receptor agonist 5′-(N-ethylcarboxamido) adenosine (NECA), and analyzed the effector cytokine secretion capacity by ELISA (FIG. 6D; FIGS. 7A-7B). NECA treatment resulted in reduced IL-2 and IFNγ production. Adenosine-dependent a2aR stimulation led to elevate intracellular 3′,5′-cyclic adenosine monophosphate (cAMP) which in turn inhibited the NF-kB pathway and cell proliferation (FIG. 6A). To evaluate whether adenosine suppression of CAR T cells worked through the same mechanisms, an NF-κB activation reporter was constructed by placing the Green Fluorescence Protein (GFP) gene downstream of NF-κB response elements. HA CAR-T cells were co-transduced with this NF-kB-GFP reporter. Upon activation with plate-bound idiotype, NECA diminished activation of the NF-kB-GFP reporter (FIG. 7C). This inhibitory effect on activation was prevented by addition of the selective A2aR competitive antagonist (iA2aR), confirming A2a receptor (A2aR) involvement in the suppressive effect of NECA on HA CAR-T cells.

These data illustrated that exhausted CAR T cells expressed active CD39 and CD73 on their surface, which resulted in increased capability of generating adenosine, and that adenosine in turn exerted suppressive effects on CAR T cell function and proliferation in an A2a receptor-mediated manner. To test this hypothesis that adenosine production was responsible for the suppressive function of exhausted CAR T cells over neighboring cells, whether HA CAR-T cells were able to produce adenosine in an autocrine manner was investigated (FIG. 7D). Next, CD19 CAR T cells that were pre-incubated with A2aR inhibitor were activated in the presence or absence of (CD8) HA CAR T cells. In agreement with the hypothesis, blocking A2a receptor (A2aR) on CD19 CAR T cells or knocking-out CD39 on HA CAR T cells restored IL-2 production by CD19 CAR T cells in the presence of adenosine-producing HA cells (FIG. 6E).

### Example 7. Purinergic Pathway Modulates Exhausted CAR T Cells Phenotype and Function

This Example describes the results of experiments performed to demonstrate that purinergic pathway modulated exhausted CAR T cells phenotype and function.

Autocrine adenosine production, as a result of co-expression of CD39 and CD73 by HA CAR T cells, can lead not only to suppressive effects on neighboring cells, but to intrinsic suppression of CAR T cell activity, and in that context modulation of the purinergic pathway would improve CAR T cell function.

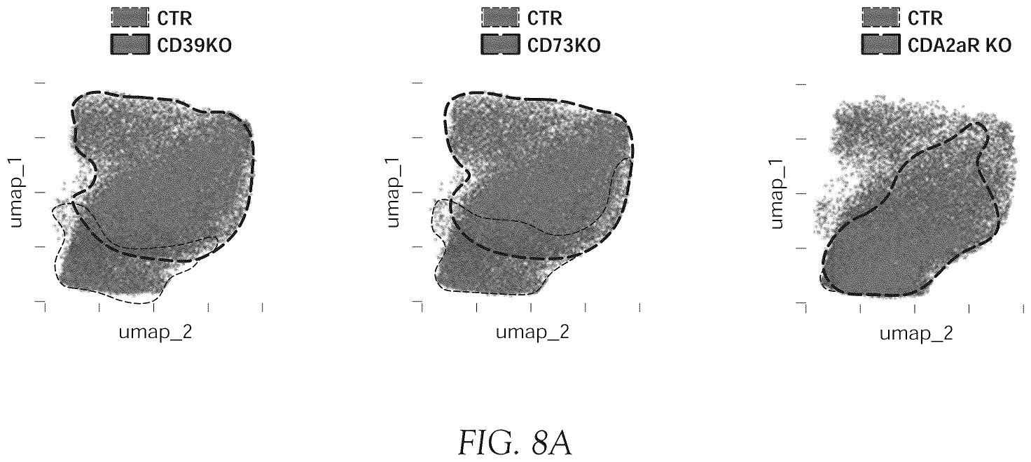

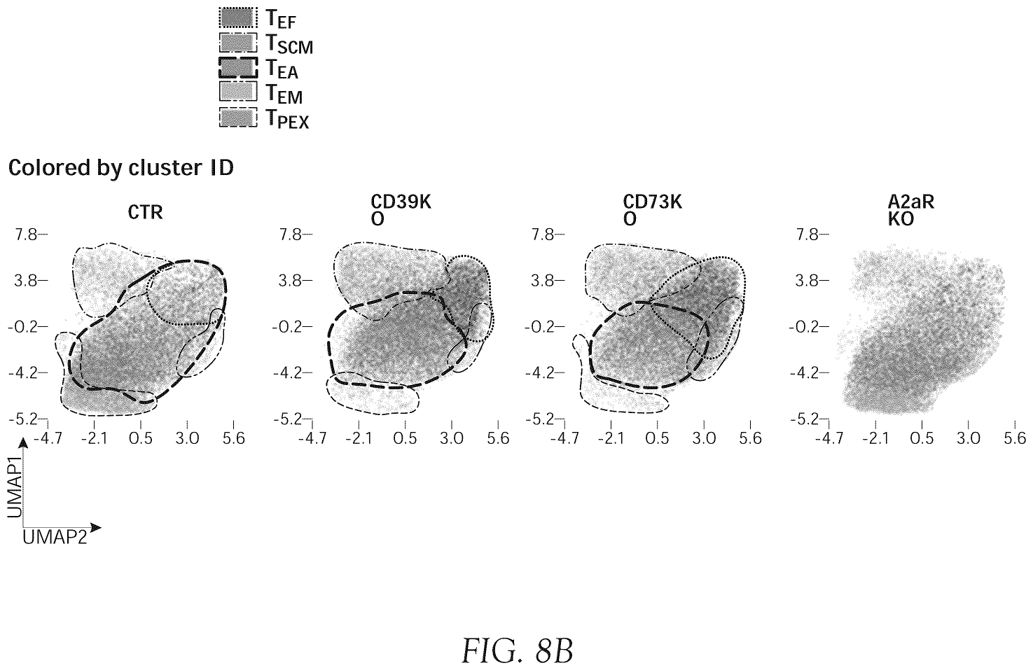

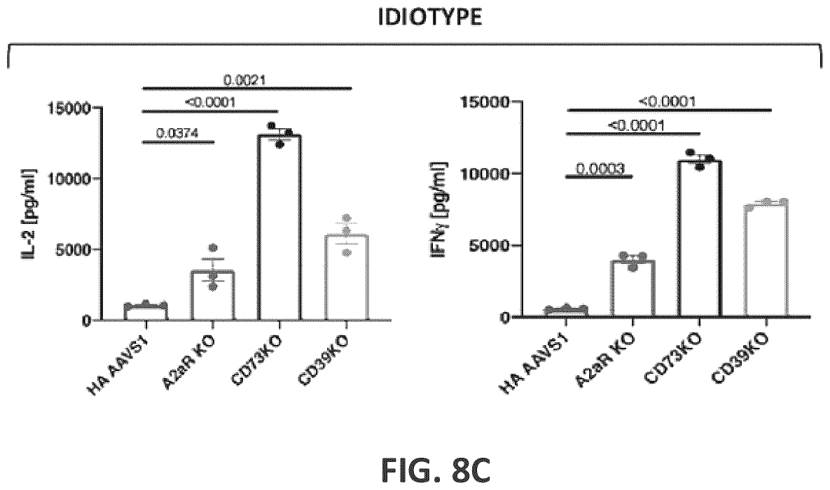

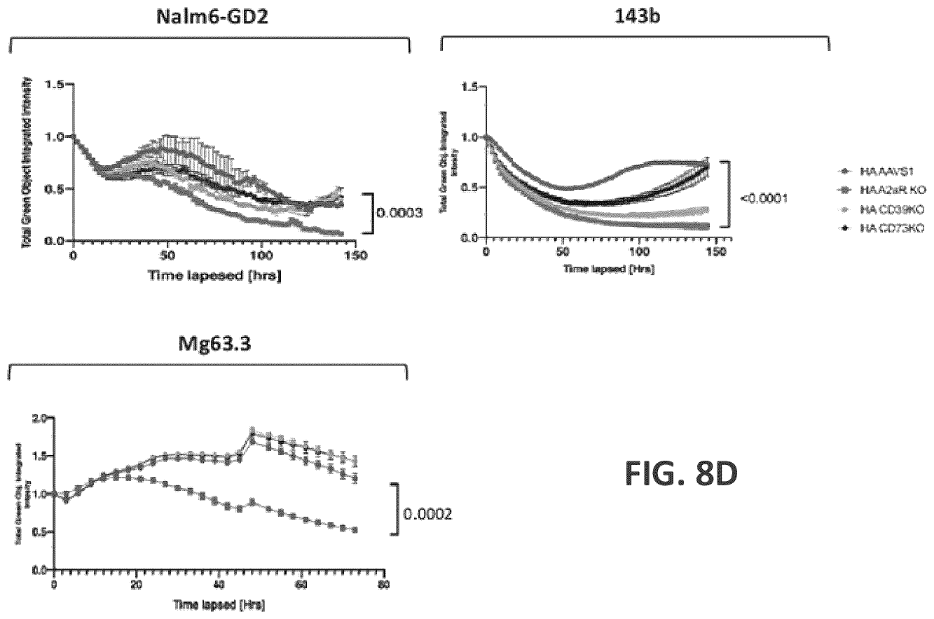

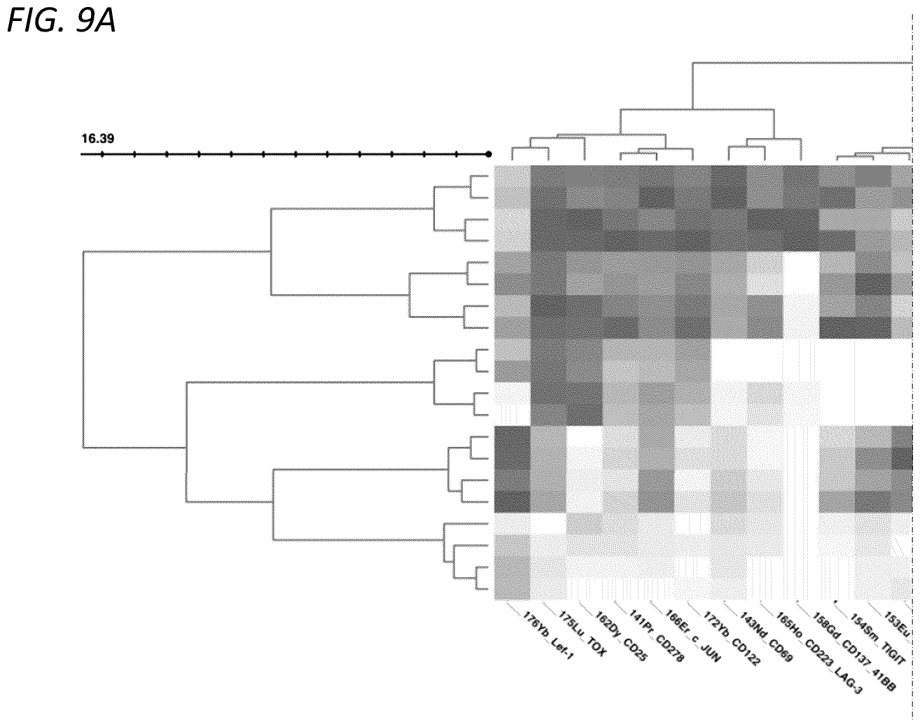

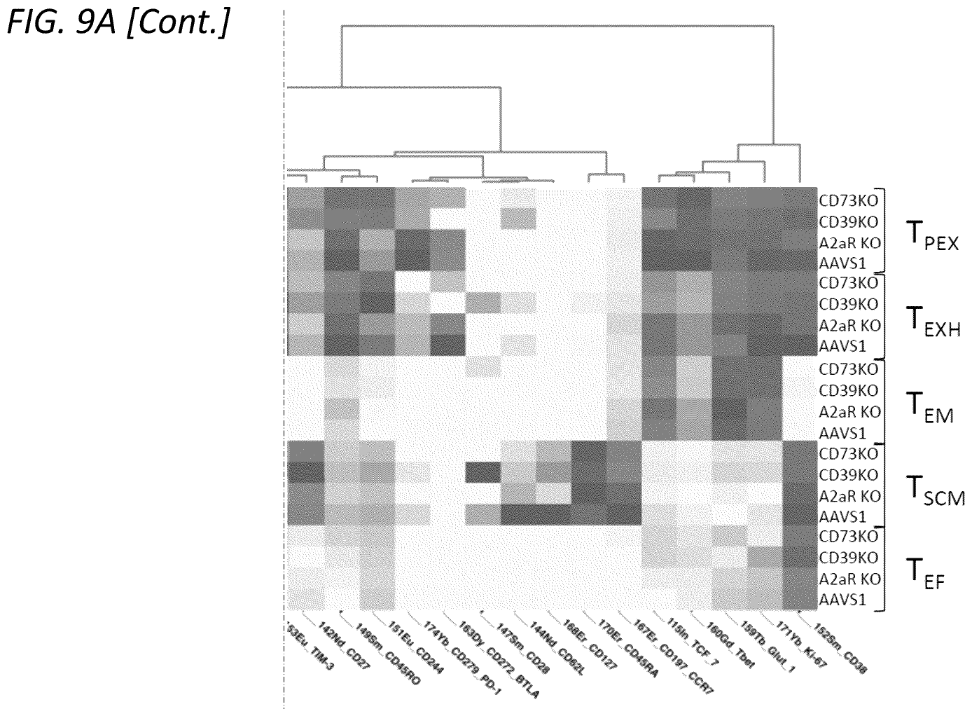

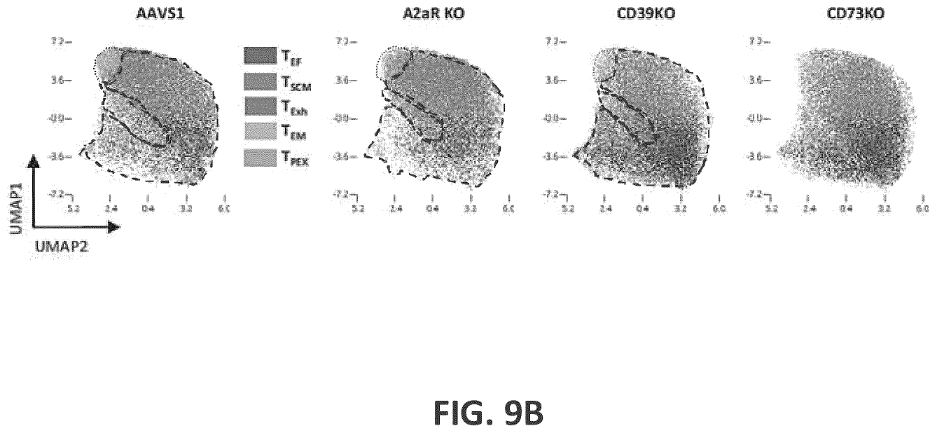

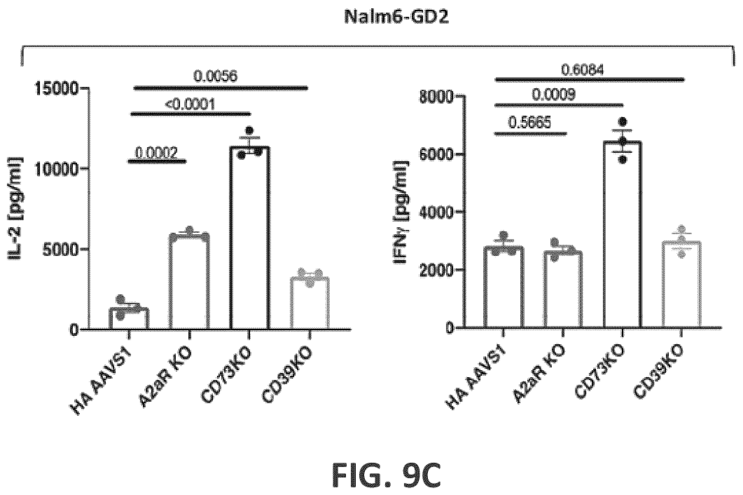

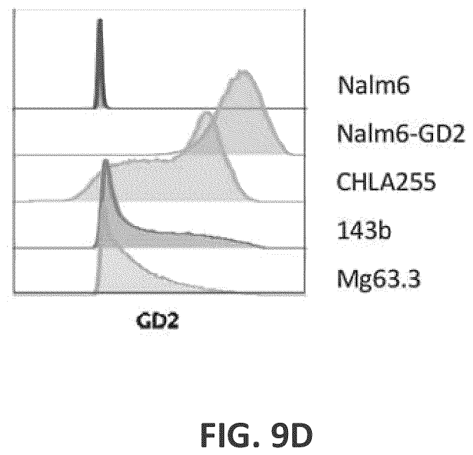

Using single-cell mass cytometry, the effects of knocking out A2aR, CD39, or CD73 were analyzed on the exhaustion phenotype of HA CAR-T cells using samples generated from 4 donors. A control was HA CAR T cells from a CRISPR knockout (KO) experiment using AAVS1 guide that targets adeno-associated virus integration site 1 with minimal risk of off-target Cas9 binding elsewhere in the genome. U-MAP analysis of data distribution showed that A2aR knock-out (KO) and control AAVS1 KO HA CAR T cells group spatially closer to each other, whereas CD39 KO and CD73 KO cells were shifted towards opposite side of the map suggesting that knock out of ectoenzymes affected phenotype of exhausted T cells to a greater extent than A2aR KO do (FIG. 8A). Further, FlowSOM analysis performed on manually gated CD4 and CD8 CAR T cells with max of number clusters set to 5 identified T cell populations (assigned as stem cell memory-T cells (T_{SCM}), effector-T cells (T_{EF}), exhausted-T cells (T_{Exh}), progenitor of exhausted-T cells (T_{PE}X) and effector memory-like T cells (T_{EM})) (FIG. 8B). This strategy reveled a significant shift of phenotype after knocking out CD39 or CD73 characterized by an increase in frequency of T_{SCM}- and effector-like CAR T cells. In contrast, A2aR KO did not cause changes in HA CAR T cell phenotype. The phenotypic characteristics of the clusters, ordered according to their lineage and the median expression of every marker, were displayed in a heatmap (FIG. 9A). Similar trend in changes in the CAR T cell phenotype has been observed for CD4+ CAR T cells (FIG. 9B).

To evaluate if those phenotypical changes translated into differences in function, IL-2 and IFNγ production of each KO HA CAR-T cells were compared upon stimulation with idiotype. Notably, both cytokine levels were increased in A2aR KO, CD39 KO and CD73 KO compared to AAVS1 KO control, although CD73KO and CD39KO showed greater increase in cytokine secretion (FIG. 8C). This trend was also observed when KO HA CAR-T cells were stimulated with Nalm6-GD2 cells, instead of idiotype (FIG. 9C). To further characterize the effect of KO components of the purinergic pathway on exhausted CAR-T cell function, the cytolytic capacity was measured against different tumor cell lines expressing different levels of GD2 on the surface (FIG. 9D). Notably, only A2aR KO improved cytotoxic function of HA CAR T cells (FIG. 8D). These results illustrated that although CD39 and CD73 KO increased cytokine secretion against tumor line with expressing high GD2 density, only A2aR KO increased the activation threshold of HA CAR T cells against low antigen density.

Taken together, these results illustrated that adenosine and the pathway that regulates its production play a crucial role in modulating CAR T cell response and phenotype.

### Example 8. Overexpression of Transmembrane-Bound ADA Improves CAR T Cells Phenotype and Effector Function

This Example describes the results of experiments performed to demonstrate that overexpression of adenosine deaminase improved CAR T cells phenotype and effector function.

In the context of the tumor microenvironment, production of adenosine was not only regulated by CD39 and CD73 present on CAR T cells, but also expressed on the surface of cancer-associated fibroblasts, stromal, or directly on the tumor cells (FIG. 11A). While knocking out CD39 or CD73 may improve cytokine secretion in vitro, it may not be a successful approach in vivo. Similarly, although A2a receptor exhibits the highest affinity for adenosine, it is not the only adenosine receptor expressed by T cells. Therefore, an alternative approach to diminish the suppressive effect of adenosine on CAR T cells would be to overexpress the enzyme responsible for metabolizing adenosine into inosine, the adenosine deaminase (ADA). To ensure that overexpressed ADA1 would anchor on the surface of the T cell, it was fused to a transmembrane domain of CD8 (SEQ ID NO: 9). To assist with detection, a Hemagglutinin tag (HA-tag) was added to its C-terminus (FIG. 10A). It was observed that ADA1 overexpression did not affect CAR surface expression (FIG. 11B). To test the ability of ADA1 overexpression to protect CAR T cells from adenosine-mediated suppression, HA CAR T cells that produce adenosine in the presence eATP were spiked and analyzed for CD69 expression (FIG. 11C). HA CAR T cells spiked by eATP expressed CD69 at lower MFI and frequency. It was observed that this activation suppression was rescued by overexpression of ADA1.

To further analyze the effect of ADA1 overexpression on the surface of HA CAR T cell, high dimensional cyTOF analysis was performed. ADA1 overexpressing HA CAR T cells showed completely different profile of expression as compared to control sample (FIG. 10B). FlowSOM analysis in comparison with other knock out conditions revealed significant decrease in frequency of progenitor of exhaustion and as a consequence exhausted population. On the other hand, stem cell memory-like population was significantly increased only in the ADA overexpression (O/E) condition. Similar trend was observed in CD4 HA CAR T cells (FIG. 11D).

Further, transcriptome profiling showed that overexpression of ADA on HA CAR-T cells drove the biggest differences in gene expression as compared to control or CD39, CD73 or A2aR knock-out (KO) (FIG. 10C). There were more than 2,500 differentially expressed genes between ADA1 O/E and control samples as represented in volcano plot (FIG. 10D). Among the most differentially expressed, genes associated with memory phenotype and persistence were identified, such as TCF7, TL7R upregulated in ADA1-overexpressing HA CAR T cells (FIG. 10E). Genes associated with effector function such as granzyme B, IL-3, TL-5, TNFSF4 (OX40), or TNFSF11 (RANKL) were downregulated. Further, to identify the subset of genes that had the greatest contribution to the enrichment score, a leading-edge analysis on the GSEA datasets was performed. There was an upregulation of many genes involved in cell proliferation, cMYC regulated pathways, and fatty acid metabolism (FIG. 11E). This result suggested changes at the metabolic levels. Metabolic states of HA and ADA1-overexpressing CAR T cells were tested by performing Mito Stress Test using Seahorse analyzer (FIG. 10F). ADA1 HA CAR T cells exhibited higher both OCR and ECAR at the base line; however, ratio OCR/ECAR indicated higher reliance on the oxidative phosphorylation than glycolysis. Further injections of mitochondrial inhibitors showed that ADA1-overexpressing exhausted CAR T cells had increased spare respiratory capacity (SRC), a characteristic of memory T cells.

To investigate if overexpression of transmembrane-bound ADA1 could affect regulatory phenotype of HA CAR T cells, expression of Foxp3 was measured. Transmembrane-bound ADA1 overexpression significantly decreases Foxp3 frequency in exhausted HA and non-exhausted CD19 CD8 and CD4 CAR T cells (FIG. 10G; FIG. 11F). Decreased percent of Tregs in ADA1+ CAR T cells translated into increased effector function against tumor lines expressing different surface antigen density and proliferation (FIG. 1011; FIG. 11G).

Taken together, ADA1 overexpression significantly and robustly increased fitness and function of CAR T cells, both exhausted and non-exhausted, by shifting their phenotype towards memory-like cells.

### Example 9. Overexpression of Adenosine Deaminase Improves CAR T Cells Phenotype and Effector Function

This Example describes the results of additional experiments performed to determine if human ADA isoforms ADA1 and ADA2 improve CAR T cell effector function.

As reported previously and described above, there are two isoforms of human adenosine deaminases: ADA1 and ADA2. Both types catalyze the deamination of adenosine and decrease immunosuppressive signal. The ADA1 polypeptide does not include any signal sequence required for protein secretion by cells, however the ADA1 polypeptide can be attached to the cell surface via the membrane-bound CD26 protein. In contrast, ADA2 is equipped in signal peptide, which can drive its secretion outside a cell (see, e.g., Zavialov A. V. et al., Biochem. J. 391, 51-57, 2005; and Zavialov A. V. et al., Biol. Chem. 285, 12367-12377, 2010).

To determine if ADA1 and ADA2 improve CAR T cell effector function in similar way, recombinant CAR T cells were engineered to express an ADA polypeptide fused to a transmembrane domain. In these experiments, the transmembrane domain was derived from CD8. Recombinant CAR T cells expressing (i) HA, (ii) HA-ADA1-TM, or (iii) HA-ADA2-TM were stimulated with leukemia Nalm6-GD2 and 143b osteosarcoma tumor cells at day 14 post activation (see e.g., FIG. 12A).

In these experiments, CAR T cells expressing (i) HA, (ii) HA-ADA1-TM, or (iii) HA-ADA2-TM were co-cultured with Nalm6-GD2 or 143b tumor lines at 8:1 T:E ratio, at day 15 post activation. Cytotoxic function was assessed in the IncuCyte assay. Representative donor shown (n=2). It was observed that CAR T cells engineered to overexpress either one of membrane-bound ADA1 and membrane-bound ADA2 resulted in an increased tumor killing compared to the control CAR T cells expressing HA (see e.g., FIG. 12B).

Moreover, it was also observed that non-exhausted, bispecific CD19-CD22 CAR T cells expressing membrane bound ADA1 or ADA2 secreted higher levels of IL-2 and IFNγ (see e.g., FIG. 12C). In these experiments, bispecific CD19-CD22.BBz CAR T cells were stimulated with Nalm6 tumor line at day 15 post-activation. IL-2 and IFNγ secretion was assessed using ELISA. Representative donor shown (n=2).

In some further experiments, CAR T cells overexpressing either ADA1-TM or ADA2-TM were activated with Nalm6-GD2 in the presence or absence of 10 uM of adenosine deaminase inhibitor—EHNA. It was observed that both CAR T cells overexpressing either HA-ADA1-TM or HA-ADA2-TM produced more IL-2 and IFNγ as compared to control group and this increase was abrogated in the presence of EHNA (see e.g., FIG. 12D). This result indicates that elevated cytokine secretion by CAR T cells was mediated by enzymatic activity of transmembrane-bound ADA.

Additional experiments were performed to test anti-tumor potency of transmembrane-bound adenosine deaminase by using 143b solid tumor model. In these experiments, NSG mice were inoculated with 1×10⁶ of 143b osteosarcoma tumor via intramuscular injections. At Day 4 after inoculation, 10×10⁶ control Her2.BBz CAR T cells or Her2.BBz CAR T cells overexpressing ADA2-TM were injected. In these experiments, NSG mice were injected intramuscular with 1×10⁶ 143b tumor cells. At Day 4 after injection, mice were injected with 1×107 CAR T cells IV expressing (i) Her2.BBz CAR (i.e., control Her2) or (ii) Her2.BBz CAR and ADA2-TM. Tumor growth was monitored by caliper measurements. N=5 mice per group. It was observed that the group of mice injected with CAR T cells expressing ADA2-TM showed significantly lower tumor burden (see e.g., FIG. 12E).

Taken together, the experimental data described above provides the evidence that overexpression of transmembrane-bound ADA1 or transmembrane-bound ADA2 significantly improves effector function of exhausted and non-exhausted CAR T cells in vitro and as well as in vivo.

While the disclosure has been particularly shown and described with reference to specific embodiments (some of which are preferred embodiments), it should be understood by those having skill in the art that various changes in form and detail can be made therein without departing from the spirit and scope of the present disclosure as disclosed herein.

What is claimed is:

1. A chimeric polypeptide comprising:
a first amino acid sequence comprising a first polypeptide module having adenosine deaminase activity; and
a second amino acid sequence comprising a second polypeptide module capable of anchoring the adenosine deaminase activity to a surface of a T cell.

2. The chimeric polypeptide of claim 1, wherein the first polypeptide module is operably linked to the second polypeptide module.

3. The chimeric polypeptide of any one of claims 1 to 2, wherein the first polypeptide module having a human adenosine deaminase activity.

4. The chimeric polypeptide of claim 3, wherein the human adenosine deaminase activity is of ADA1, ADA2, or a functional variant of any thereof.

5. The chimeric polypeptide of claim 4, wherein the first polypeptide module comprises a amino acid sequence having at least 80% sequence identity to SEQ ID NO: 7 or SEQ ID NO: 8.

6. The chimeric polypeptide of any one of claims 1-5, wherein the second polypeptide module comprises a polypeptide transmembrane domain.

7. The chimeric polypeptide of claim 6, wherein the polypeptide transmembrane domain is derived from a CD8α, CD4, CD28, CD80, ICOS, CTLA4, PD1, PD-L1, BTLA, HVEM, CD27, 4-1BB, 4-1BBL, OX40, OX40L, DR3, GITR, CD30, SLAM, CD2, 2B4, TIM1, TIM2, TIM3, TIGIT, CD226, CD160, LAG3, LAIR1, B7-1, B7-H1, and B7-H transmembrane domain.

8. The chimeric polypeptide of claim 7, wherein the polypeptide transmembrane domain is a CD8 transmembrane domain or a functional variant thereof.

9. The chimeric polypeptide of claim 8, wherein the CD8 transmembrane domain comprises an amino acid sequence having at least 80% sequence identity to SEQ ID NO: 9.

10. A method for generating an engineered T cell with enhanced effector function, the method comprising introducing into a T cell a chimeric polypeptide according to any one of claims 1-6, or nucleic acid encoding the chimeric polypeptide.

11. The method of claim 10, wherein the introduced chimeric polypeptide results in a reduced intracellular level of adenosine in the engineered T cell compared to reference T cell that does not comprise the chimeric polypeptide.

12. The method of any one of claims 10-11, wherein the introduced chimeric polypeptide results in an enhanced effector function of the engineered T cell.

13. The method of any one of claims 10-12, further comprising introducing into the T cell at least one recombinant antigen-specific receptor.

14. The method of claim 13, wherein the at least one recombinant antigen-specific receptor comprises an engineered T cell receptor (TCR) and/or an engineered chimeric antigen receptor (CAR).

15. An engineered T cell produced by a method according to any one of claims 10-14.

16. An engineered T cell comprising a chimeric polypeptide comprising:
a first amino acid sequence comprising a first polypeptide module having adenosine deaminase activity; and
a second amino acid sequence comprising a second polypeptide module capable of anchoring the adenosine deaminase activity to a surface of a T cell.

17. The engineered T cell of any one of claims 15 to 16, wherein the T cell is a CD8+ T cytotoxic lymphocyte cell or a CD4+ T helper lymphocyte cell.

18. The engineered T cell of claim 17, wherein the CD8+ T cytotoxic lymphocyte cell is selected from the group consisting of naïve CD8+ T cells, central memory CD8+ T cells, effector memory CD8+ T cells, effector CD8+ T cells, CD8+ stem memory T cells, bulk CD8+ T cells

19. The engineered T cell of claim 17, wherein the CD4+ T helper lymphocyte cell is selected from the group consisting of naïve CD4+ T cells, central memory CD4+ T cells, effector memory CD4+ T cells, effector CD4+ T cells, CD4+ stem memory T cells, and bulk CD4+ T cells.

20. The engineered T cell of any one of claims 15-19, wherein the T cell is an exhausted T cell or a non-exhausted T cell.

21. The engineered T cell of any one of claims 15 to 20, wherein the T cell was obtained leukapheresis of a sample obtained from a subject.

22. A cell culture comprising at least one engineered T cell according to any one of claims 15-20, and a culture medium.

23. A pharmaceutical composition comprising an engineered T cell according to any one of claims 15-21, and a pharmaceutically acceptable excipient.

24. A method for preventing and/or treating a health condition in a subject in need thereof, the method comprising administering to the subject a composition comprising:
(a) at least one engineered T cells according to anyone of claims 15-21; and/or
(b) a pharmaceutical composition according to claim 23.

25. The method of claim 24, wherein the health condition is a proliferative disease, an autoimmune disease, or a chronic infection.

26. The method of claim 25, wherein the proliferative disease is a cancer.

27. The method of any one of claims 24-25, wherein the subject is a mammalian subject.

28. The method of claim 27, wherein the mammalian subject is a human subject.

29. The method of any one of claims 24-28, wherein the engineered T cells are autologous to the subject.

30. The method of any one of claims 24-29, wherein the engineered T cells are obtained from tumor infiltrating lymphocytes (TILs) or peripheral blood mononuclear cells (PBMCs).

31. The method of any one of claims 24-30, wherein the administered composition inhibits adenosine-mediated immunosuppression in the subject.

32. The method of any one of claims 24-31, wherein the administered composition confers an enhanced effector function of the engineered T cells.

33. The method of claim 32, wherein the enhanced effector function of the engineered T cells is selected from the group consisting of growth rate (proliferation), death rate, death rate type, target cell inhibition (cytotoxicity), cluster of differentiation change, macrophage activation, B cell activation, cytokine production, in vivo persistence, and increased spare respiratory capacity.

34. The method of any one of claims 32-33, wherein the enhanced effector function comprises increased production of one or more cytokines.

35. The method of claim 35, wherein the one or more cytokines comprises e.g. interferon gamma (INFγ), tumor-necrosis factor α (TNFα), and/or interleukin-2 (IL-2).

36. The method of any one of claims 24-34, wherein the composition is administered to the subject individually (monotherapy) or in combination with a second therapy, wherein the second therapy is selected from the group consisting of chemotherapy, radiotherapy, immunotherapy, hormonal therapy, toxin therapy, or surgery.

37. A kit for the prevention and/or treatment of a condition in a subject in need thereof, the kit comprising:
(a) a chimeric polypeptide according to any one of any one of claims 1-9;
(b) nucleic acid encoding the chimeric polypeptide of (a);
(c) at least one engineered T cells according to any one of claims 15-21; and/or
(d) a pharmaceutical composition according to claim 23.
